(19) United States
(12) Patent Application Publication
(10) Pub. No.: US 2013/0259830 A1
(43) Pub. Date: Oct. 3, 2013

(54) CRTH2 MODULATORS

(71) Applicants: Timothy Barden, Salem, MA (US); Colleen Hudson, Malden, MA (US); James Jia, Somerville, MA (US); Charles Kim, Cambridge, MA (US); Regina Graul, Duxbury, MA (US); Ara Mermerian, Somerville, MA (US); Joel Moore, Lexington, MA (US); Kevin Sprott, Needham, MA (US); Yueh-tyng Chien, Newton, MA (US)

(72) Inventors: Timothy Barden, Salem, MA (US); Colleen Hudson, Malden, MA (US); James Jia, Somerville, MA (US); Charles Kim, Cambridge, MA (US); Regina Graul, Duxbury, MA (US); Ara Mermerian, Somerville, MA (US); Joel Moore, Lexington, MA (US); Kevin Sprott, Needham, MA (US); Yueh-tyng Chien, Newton, MA (US)

(73) Assignee: Ironwood Pharmaceuticals, Inc., Cambridge, MA (US)

(21) Appl. No.: 13/809,548

(22) PCT Filed: Jun. 24, 2011
(86) PCT No.: PCT/US2011/041772

§ 371 Date: Apr. 25, 2013

Related U.S. Application Data
(60) Provisional application No. 61/363,501, filed on Jul. 12, 2010.

(51) Int. Cl. C07D 209/14; A61K 31/4439; C07D 409/06; A61K 31/405; A61K 45/06; A61K 31/496; C07D 209/30; C07D 471/04; A61K 31/437; A61K 31/5377; C07D 401/06; C07D 491/052

(52) U.S. Cl. CPC C07D 209/14; C07D 401/06; A61K 31/4439; C07D 409/06; A61K 31/405; C07D 491/052; A61K 31/496; C07D 209/30; C07D 471/04; A61K 31/437; A61K 31/5377; A61K 45/06
USPC 424/85.6; 546/277.4; 514/339; 548/468; 514/414; 544/373; 514/254.08; 548/484; 514/424; 546/113; 514/300; 544/127; 514/234.5; 544/144; 514/235.2; 548/465; 424/158.1; 514/171; 514/23; 548/511; 514/415

(57) ABSTRACT

Modulators of CRTH2, particularly antagonists of CRTH2, that are useful for treating various disorders, including asthma and respiratory disorders are disclosed. The compounds fall within a genus described by Formula I:

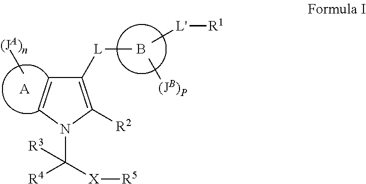

This patent application claims the benefit of U.S. Provisional Application No. 61/363,501 filed Jul. 12, 2010, the disclosure of which is hereby incorporated by reference.

### FIELD OF THE INVENTION

This disclosure relates to modulators of chemoattractant receptor-homologous molecule expressed on T helper type 2 cells (CRTH2), particularly CRTH2 antagonists that are useful for treating various disorders, including asthma and allergic and respiratory disorders.

### BACKGROUND

CRTH2 is a Gaᵢ protein-coupled receptor involved in both mediating PGD2-induced chemoattraction and in activation of specific cell types involved in allergic inflammation. CRTH2 is expressed by Th2 cells, eosinophils and basophils, but not by Th1 cells, B cells or NK cells. PGD2 is produced by allergen-activated mast cells and has been implicated in various allergic diseases as a pro-inflammatory mediator, such as asthma, rhinitis and allergies. Thus, blocking binding of PGD₂ to CRTH2 may be a useful therapeutic strategy for treatment of such diseases.

CRTH2 agonists activate eosinophils, basophils and Th2 cells in vitro, resulting in induction of actin polymerization, calcium influx, CD11b expression and chemotaxis. Injection of a CRTH2 agonist in vivo can elicit transient recruitment of eosinophils from bone marrow into the blood. A genetic study of African American and Chinese cohorts found that polymorphisms in CRTH2 were tightly associated with asthma susceptibility. Thus, it has been suggested that modulators of CRTH2, particularly CRTH2 inhibitors, may be useful in the prevention and/or treatment of allergic asthma and other allergic disorders as recruitment and/or activation of eosinophils, basophils and Th2 cells is a prominent feature of the changes that occur in the asthmatic lung. Similar activation of these cell types, or subsets thereof, is believed to play an important role in the etiology of other diseases, including eosinophilic esophagitis and atopic dermatitis. This fact, combined with the fact that CRTH2 mediates PGD₂-induced chemotaxis, suggests that compounds that alter chemotaxis by inhibiting CRTH2 activity could be useful in controlling various diseases and disorders, including, without limitation, allergic asthma, chronic airway inflammation, atopic dermatitis, chronic obstructive pulmonary disease (COPD), and/or eosinophilic esophagitis.

Compounds that alter chemotaxis by inhibiting CRTH2 activity could also be useful in controlling allergic rhinitis, which is classified as either seasonal (SAR) or perennial (PAR) depending upon the type of trigger and duration of symptoms. SAR symptoms occur in the spring, summer and/or early fall and can be triggered by outdoor allergens such as airborne tree, grass and weed pollens while PAR is usually persistent and chronic with symptoms occurring year-round and is commonly associated with indoor allergens such as dust mites, animal dander and/or mold spores. Symptoms of allergic rhinitis may include runny nose, nasal itching, sneezing, watery eyes and nasal congestion.

CRTH2 agonists can induce desensitization of the cell system by promoting internalization and down regulation of the cell surface receptor. For example, certain CRTH2 agonists can induce desensitization of PGD₂-responsive cells to subsequent activation by a CRTH2 agonist. Therefore, CRTH2 modulators that are CRTH2 agonists may be therapeutically useful because they can cause the desensitization of PGD₂-responsive cells. Importantly, CRTH2 agonists may also cause cross-desensitization. Cross-desensitization, which can occur in many cell-signaling systems, refers to a phenomenon whereby an agonist for one receptor can reduce or eliminate sensitivity of a cell type to an unrelated agonist/receptor signaling system. For example, treatment with the CRTH2 agonist indomethacin reduces expression of CCR3, the receptor for the chemoattractant, eotaxin.

CRTH2 is also found on cell types outside the immune system, including spinal cord neurons and brain. PGD₂ activation of CRTH2, e.g., during inflammation, can lead to hyperalgesia, allodynia and neuropathic pain. Thus, inhibitors of CRTH2 may be used to treat hyperalgesia, allodynia and neuropathic pain.

Accordingly, there is a need to develop inhibitors of CRTH2, which may be useful in preventing and/or treating disorders such as allergic rhinitis, asthma, chronic airway inflammation, atopic dermatitis, chronic obstructive pulmonary disease (COPD), eosinophilic esophagitis and/or neuropathic pain.

### SUMMARY

In a first aspect, compounds disclosed herein and pharmaceutically acceptable salts thereof, are effective as CRTH2 modulators. These compounds are represented by Formula I:

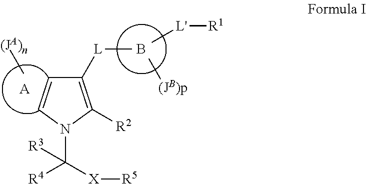

wherein:

Ring A is a 5 to 10-membered cycloaliphatic ring or a 4 to 10-membered heterocycle; wherein said heterocycle contains from 1 to 3 ring heteroatoms independently selected from N, O or S;

n is an integer selected from 0 to 6;

each occurrence of J^{A} is independently selected from halogen, C₁₋₄ alkyl, C₁₋₄ haloalkyl, C₁₋₄ alkoxy, C₁₋₄ haloalkoxy, —CN, —OH, oxo, ═NOR⁶, —NH(C₁₋₄alkyl), —N(C₁₋₄ alkyl)₂ or —NH₂;

R⁶ is selected from C₁₋₆ alkyl or —(C₁₋₆ alkyl)-(C₆₋₁₀ aryl);

Ring B is a monocyclic or bicyclic ring selected from phenyl or a 5 to 10-membered heteroaryl ring; wherein said heteroaryl ring contains 1 to 3 ring heteroatoms independently selected from N, O or S;

p is an integer selected from 0 to 2;

each occurrence of J^{B} is independently selected from halogen, —NO₂, —CN, —OH, —SH, —NH₂, C₁₋₄ alkyl, C₁₋₄ haloalkyl, —O(C₁₋₄ alkyl), —O(C₁₋₄haloalkyl), —S(C₁₋₄ alkyl), —NH(C₁₋₄ alkyl) or —N(C₁₋₄ alkyl)₂;

L is a linker selected from a methylene, —O—, —S(O)ₘ— or —NR⁹—; wherein said methylene is optionally and independently substituted with 1 or 2 instances of halogen or C₁₋₄ alkyl; wherein m is an integer selected from 0 to 2;

R⁹ is selected from hydrogen or C₁₋₄ alkyl;

L′ is a linker selected from —Y—SO₂—, —Z—C(O)—, —C(O)—Z— or a C₁₋₂ alkylene chain; wherein:

when ring B is phenyl, then Y is selected from a methylene or —NR¹⁰— and Z is selected from a single bond, a methylene or —NR¹⁰—; or

alternatively, when ring B is phenyl, L′ and R¹ together form a 5 to 6-membered heterocyclic ring which is linked to the phenyl ring B through a N ring atom; wherein said 5 to 6 membered heterocyclic ring optionally contains a heteroatom selected from N, O or S in addition to the N ring atom linked to the phenyl ring B; wherein said heteroaryl ring formed by L′ and R¹ is optionally and independently substituted by up to two instances of R⁸; or

alternatively, when ring B is a 5 to 10-membered heteroaryl ring, then Y is selected from a single bond, a methylene or —NR¹⁰— and Z is selected from a single bond, a methylene or —NR¹⁰—;

R¹⁰ is selected from hydrogen, a C₁₋₆ alkyl or a —(C₁₋₆ alkyl)-(C₆₋₁₀ aryl) group;

R¹ is selected from a C₁₋₆ aliphatic, C₁₋₆ haloaliphatic or a monocyclic ring selected from a 3 to 8-membered cycloaliphatic, a phenyl ring, a 5 to 6-membered heteroaryl or a 4 to 8-membered heterocycle; wherein said heteroaryl or heterocycle contains from 1 to 3 ring heteroatoms independently selected from N, O or S; and wherein when R¹ is a ring, it is optionally and independently substituted with up to three instances of R⁸;

each occurrence of R⁸ is independently selected from halogen, C₁₋₄ alkyl, C₁₋₄ haloalkyl, a 6 to 10 membered arylalkoxy group, C₁₋₄ alkoxy, —C(O)O(C₁₋₄ alkyl), —C(O)(C₁₋₄ alkyl), —CN, —OH, —NH₂, —NH(C₁₋₄ alkyl) or —N(C₁₋₄ alkyl)₂;

R² is selected from hydrogen, halogen, C₁₋₄ alkyl, C₁₋₄ haloalkyl, C₁₋₄ alkoxy, C₁₋₄ haloalkoxy, —CN or —OH;

R³ and R⁴ are each independently selected from hydrogen, deuterium, halogen, C₁₋₆ alkyl or C₁₋₆ haloalkyl; or R³ and R⁴ taken together with the atom to which they are attached form a C₃₋₇ cycloalkyl ring;

X is selected from a direct single bond or a methylene linker, wherein said methylene is optionally substituted with up to two identical or different instances of halogen, C₁₋₄ alkyl or C₁₋₄ haloalkyl;

R⁵ is selected from —C(O)OR⁷, —C(O)O—R¹¹, —C(O)N(R⁷)₂, —C(O)NOR⁷ or —C(O)NSR⁷;

each occurrence of R⁷ is independently selected from hydrogen or C₁₋₄ alkyl; and

each occurrence of R¹¹ is independently selected form a —(C₁₋₆ alkyl)-(C₆₋₁₀ heteroaryl), a —(C₁₋₆ alkyl)-(C₆₋₁₀ heterocyclyl) or a —(C₁₋₆ alkyl)-(C₆₋₁₀ aryl) wherein each of said heteroaryl and heterocycle contains 1 to 3 ring heteroatoms independently selected from N, O or S, and wherein R¹¹ is optionally and independently substituted with up to three instances of R⁸.

In another aspect, compositions comprising a pharmaceutically acceptable carrier and a compound described above, or a pharmaceutically acceptable salt thereof, are provided.

In another aspect, a method for preventing or treating a disease involving a CRTH2 receptor or of lessening the severity of a disease involving a CRTH2 receptor, in a patient suffering from such disease is provided. The method comprises administering to the patient a therapeutically or prophylactically effective amount of a compound described above, or a pharmaceutically acceptable salt thereof. Typical diseases that involve the CRTH2 receptor and that may be treated or prevented with the compounds described above, or a pharmaceutically acceptable salt thereof include, without limitation, asthma, allergic rhinitis and chronic obstructive pulmonary disease (COPD).

In another aspect, a method for treating a patient suffering from a disease involving the CRTH2 receptor comprising administering a therapeutically or prophylactically effective amount of a CRTH2 inhibitor compound described herein, or a pharmaceutically acceptable salt thereof, to the patient in combination with one or more therapeutic agents is provided. In another aspect, pharmaceutical compositions comprising at least one compound represented by one of Formulae I to XXIII, or at least one of Compound Nos. I-1 to I-40 and I-42 to I-57, of Table I, or a pharmaceutically acceptable salt thereof, and one or more therapeutic agents, for use in the treatment or prevention of a disease involving the CRTH2 receptor are provided. Methods for the use of at least one compound represented by one of Formulae I to XXIII, or at least one of Compound Nos. I-1 to I-40 and I-42 to I-57 of Table I, for the manufacture of medicaments for the treatment and/or prevention of a disease involving the CRTH2 receptor are also provided.

### DETAILED DESCRIPTION OF THE INVENTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

For purposes of this disclosure, the chemical elements are identified in accordance with the Periodic Table of the Elements, CAS version, and the Handbook of Chemistry and Physics, 75.sup.th Ed. 1994. Additionally, general principles of organic chemistry are described in “Organic Chemistry”, Thomas Sorrell, University Science Books, Sausalito: 1999, and “March's Advanced Organic Chemistry”, 5.sup.th Ed., Smith, M. B. and March, J., eds. John Wiley & Sons, New York: 2001, which are herein incorporated by reference in their entirety.

As described herein, compounds of the invention may optionally be substituted with one or more substituents, such as illustrated generally below, or as exemplified by particular classes, subclasses, and species of the invention. The phrase “optionally substituted” is used interchangeably with the phrase “substituted or unsubstituted.” In general, the term “substituted”, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. If a substituent radical or structure is not identified or defined as “optionally substituted”, the substituent radical or structure is not substituted. As it will be apparent to one of ordinary skill in the art, groups such as —H, halogen, —NO₂, —CN, —OH, —NH₂ or —OCF₃ would not be substitutable groups.

The phrase “up to”, as used herein, refers to zero or any integer number that is equal or less than the number following the phrase. For example, “up to 3” means any one of 0, 1, 2, or 3. As described herein, a specified number range of atoms includes any integer therein. For example, a group having from 1-4 atoms could have 1, 2, 3 or 4 atoms.

When any variable occurs more than one time at any position, its definition on each occurrence is independent from every other occurrence, unless otherwise indicated.

Selection of substituents and combinations envisioned by this disclosure are only those that result in the formation of stable or chemically feasible compounds. Such choices and combinations will be apparent to those of ordinary sill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. In some embodiments, a stable compound or chemically feasible compound is one that is not substantially altered when kept at a temperature of 25° C. or less, in the absence of moisture or other chemically reactive conditions, for at least a week.

A compound, such as the compounds of the invention or other compounds herein disclosed may be present in its free form (e.g. an amorphous form, a crystalline form or polymorphs). Under certain conditions, compounds may also form salts.

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are within the scope of the disclosure.

The present disclosure also embraces isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The term “aliphatic” or “aliphatic group” or “aliphatic radical”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”. In some instances the term can be used in the phrase “aromatic carbocycle”, and in this case it refers to an “aryl group” as defined below.

The term “cycloaliphatic” or “cycloaliphatic ring” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, spiro or bridged.

In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3-7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

“Heterocycle” (or “heterocyclyl” or “heterocyclic), as used herein, refers to a ring system in which one or more ring members are an independently selected heteroatom, which is completely saturated or that contains one or more units of unsaturation but which is not aromatic, and which has a single point of attachment to the rest of the molecule. Unless otherwise specified, through this disclosure, heterocycle is used as a synonym of “non-aromatic heterocycle”). In some instances the term can be used in the phrase “aromatic heterocycle”, and in this case it refers to a “heteroaryl group” as defined below. The term heterocycle also includes fused, Spiro or bridged heterocyclic ring systems. Unless otherwise specified, a heterocycle may be monocyclic, bicyclic or tricyclic. In some embodiments, the heterocycle has 3-18 ring members in which one or more ring members is a heteroatom independently selected from oxygen, sulfur or nitrogen, and each ring in the system contains 3 to 7 ring members. In other embodiments, a heterocycle may be a monocycle having 3-7 ring members (2-6 carbon atoms and 1-4 heteroatoms) or a bicycle having 7-10 ring members (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is in the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl.

The term “arylalkyl” refers to a substituent in which an aryl residue is attached to the parent structure through alkyl. Examples of arylalkyl are benzyl, phenethyl and the like. Heteroarylalkyl refers to a substituent in which a heteroaryl residue is attached to the parent structure through alkyl. In one embodiment, the alkyl group of an arylalkyl or a heteroarylalkyl is an alkyl group of from 1 to 6 carbons. Examples of heteroarylalkyl include, e.g., pyridinylmethyl, pyrimidinylethyl and the like.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6,5 heteroaromatic system, as used herein, for example, is a six membered heteroaromatic ring fused to a second five membered ring wherein the radical or point of attachment is on the six membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tri-cyclic ring systems including cycloaliphatic, heterocyclic, aryl or heteroaryl, each of which has been previously defined.

“Fused” bicyclic ring systems comprise two rings which share two adjoining ring atoms. For nomenclature purposes, two rings that have two atoms and one bond in common may be regarded as being derived from the two rings as separate entities. For example, Formula D10 below can be described as a cyclohexane ring fused to a pyrrole ring.

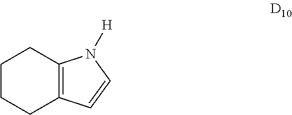

“Bridged” bicyclic ring systems comprise two rings which share three or four adjacent ring atoms. As used herein, the term “bridge” refers to a bond or an atom or a chain of atoms connecting two different parts of a molecule. The two atoms that are connected through the bridge (usually but not always, two tertiary carbon atoms) are referred to as “bridgeheads”. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl.

“Spiro” bicyclic ring systems share only one ring atom (usually a quaternary carbon atom).

The term “ring atom” refers to an atom such as C, N, O or S that is part of the ring of an aromatic group, a cycloaliphatic group or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example N (as in 3,4-dihydro-2H-pyrrolyl), NH (as in pyrrolidinyl) or NR⁺ (as in N-substituted pyrrolidinyl).

In some embodiments, two independent occurrences of a variable may be taken together with the atom(s) to which each variable is bound to form a 5-8-membered, heterocyclyl, aryl, or heteroaryl ring or a 3-8-membered cycloalkyl ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of R^{o} as in Formula D1:

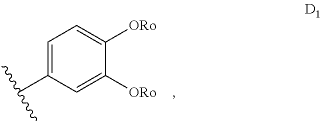

these two occurrences of R^{o} are taken together with the oxygen atoms to which they are bound to form a fused 6-membered oxygen containing ring as in Formula D2:

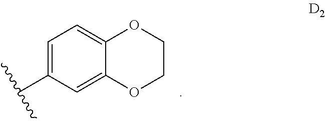 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

In some embodiments, an alkyl or aliphatic chain can be optionally interrupted with another atom or group. This means that a methylene unit of the alkyl or aliphatic chain can optionally be replaced with said other atom or group. Unless otherwise specified, the optional replacements form a chemically stable compound. Optional interruptions can occur both within the chain and/or at either end of the chain; i.e. both at the point of attachment(s) to the rest of the molecule and/or at the terminal end. Two optional replacements can also be adjacent to each other within a chain so long as it results in a chemically stable compound. Unless otherwise specified, if the replacement or interruption occurs at a terminal end of the chain, the replacement atom is bound to a H on the terminal end. For example, if —CH₂CH₂CH₃ were optionally interrupted with —O—, the resulting compound could be —OCH₂CH₃, —CH₂OCH₃, or —CH₂CH₂OH. In another example, if the divalent linker —CH₂CH₂CH₂— were optionally interrupted with —O—, the resulting compound could be —OCH₂CH₂—, —CH₂OCH₂—, or —CH₂CH₂O—. The optional replacements can also completely replace all of the carbon atoms in a chain. For example, a C₃ aliphatic can be optionally replaced by —N(R^{$})—, —C(O)—, and —N(R^{$})— to form —N(R^{$})C(O)N(R^{$})— (a urea).

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent at the end of the substituent bound to the rest of the chemical structure. Alkylcarboxy (e.g., alkyl-C(O)O— or)alkyl-O(CO)—) and alkylcarboxyaryl (e.g., alkyl-C(O)O-aryl- or alkyl-O(CO)-aryl-) are examples of carboxy groups used internally.

As described herein, a bond drawn from a substituent to the center of one ring within a multiple-ring system (as shown below), represents substitution of the substituent at any substitutable position in any of the rings within the multiple ring system. For example, Formula D3 represents possible substitution in any of the positions shown in Formula D4:

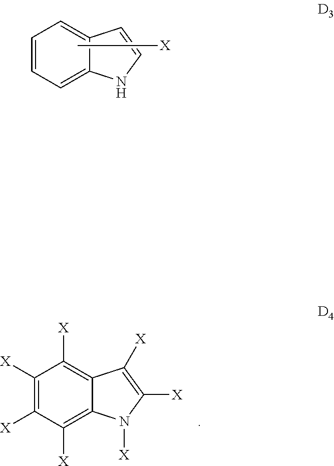

This also applies to multiple ring systems fused to optional ring systems (which would be represented by dotted lines). For example, in Formula D5, X is an optional substituent both for ring A and ring B.

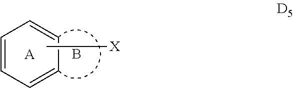

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

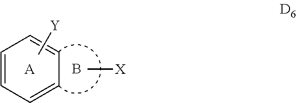

As used herein, the terms “alkoxy” or “alkylthio” refer to an alkyl group, as previously defined, attached to the molecule, or to another chain or ring, through an oxygen (“alkoxy” i.e, —O-alkyl) or a sulfur (“alkylthio” i.e., S-alkyl) atom.

The terms Cₙ₋ₘ “alkoxyalkyl”, Cₙ₋ₘ “alkoxyalkenyl”, Cₙ₋ₘ “alkoxyaliphatic”, and Cₙ₋ₘ “alkoxyalkoxy” mean alkyl, alkenyl, aliphatic or alkoxy, as the case may be, substituted with one or more alkoxy groups, wherein the combined total number of carbons of the alkyl and alkoxy groups, alkenyl and alkoxy groups, aliphatic and alkoxy groups or alkoxy and alkoxy groups, combined, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —CH₂CH₂CH₂OCH₃.

When the moieties described in the preceding paragraph are optionally substituted, they can be substituted in either or both of the portions on either side of the oxygen or sulfur. For example, an optionally substituted C₄ alkoxyalkyl could be, for instance, —CH₂CH₂OCH(Me)CH₃ or —CH₂(OH)OCH₂CH₂CH₃; a C₅ alkoxyalkenyl could be, for instance, —CH═CHOCH₂CH₂CH₃ or —CH═CHCH₂OCH₂CH₃.

The terms aryloxy, arylthio, benzyloxy or benzylthio, refer to an aryl or benzyl group attached to the molecule, or to another chain or ring, through an oxygen (“aryloxy”, benzyloxy e.g., —O-Ph, —OCH₂Ph) or sulfur (“arylthio” e.g., —S-Ph, —S—CH₂Ph) atom. Further, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or aliphatic will be indicated separately. Thus, a 5-6-membered aryloxy(C₁₋₄alkyl) is a 5-6 membered aryl ring, attached via an oxygen atom to a C₁₋₄ alkyl chain which, in turn, is attached to the rest of the molecule via the terminal carbon of the C₁₋₄ alkyl chain.

As used herein, the terms “halogen” or “halo” mean fluorine, chlorine, bromine or iodine.

In one embodiment, halogen may be fluorine or chlorine.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example a C₁₋₃ haloalkyl could be CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN (or —C≡N).

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. For example a C₁₋₃ cyanoalkyl could be —C(CN)₂CH₂CH₃ and a C₁₋₂ cyanoalkenyl could be ═CHC(CN)H₂.

As used herein, an “amino” group refers to —NH₂.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups. For example a C₁₋₃ aminoalkyl could be —CH(NH₂)CH₂CH₂NH₂ and a C₁₋₂ aminoalkoxy could be —OCH₂CH₂NH₂.

The term “hydroxyl” or “hydroxy” refers to —OH.

The terms “hydroxyalkyl”, “hydroxyalkenyl”, “hydroxyaliphatic”, and “hydroxyalkoxy” mean alkyl, alkenyl, aliphatic or alkoxy, as the case may be, substituted with one or more —OH groups. For example a C₁₋₃ hydroxyalkyl could be —CH₂(CH₂OH)CH₃ and a C₄ hydroxyalkoxy could be —OCH₂C(CH₃)(OH)CH₃.

As used herein, a “carbonyl”, used alone or in connection with another group refers to —C(O)— or —C(O)H. For example, as used herein, an “alkoxycarbonyl,” refers to a group such as —C(O)O(alkyl).

As used herein, an “oxo” refers to wherein oxo is usually, but not always, attached to a carbon atom. An aliphatic chain can be optionally interrupted by a carbonyl group or can optionally be substituted by an oxo group, and both expressions refer to the same: e.g. —CH₂—C(O)—CH₃.

As used herein, in the context of resin chemistry (e.g. using solid resins or soluble resins or beads), the term “linker” refers to a bifunctional chemical moiety attaching a compound to a solid support or soluble support.

In all other situations, a “linker”, as used herein, refers to a divalent group in which the two free valences are on different atoms (e.g. carbon or heteroatom) or are on the same atom but can be substituted by two different substituents. For example, a methylene group can be C₁ alkyl linker (—CH₂—) which can be substituted by two different groups, one for each of the free valences (e.g. as in Ph-CH₂-Ph, wherein methylene acts as a linker between two phenyl rings). Ethylene can be C₂ alkyl linker (—CH₂CH₂—) wherein the two free valences are on different atoms. The amide group, for example, can act as a linker when placed in an internal position of a chain (e.g. —CONH—). A linker can be the result of interrupting an aliphatic chain by certain functional groups or of replacing methylene units on said chain by said functional groups. E.g. a linker can be a C₁₋₆ aliphatic chain in which up to two methylene units are substituted by —C(O)— or —NH— (as in —CH₂—NH—CH₂—C(O)—CH₂— or —CH₂—NH—C(O)—CH₂—). An alternative way to define the same —CH₂—NH—CH₂—C(O)—CH₂— and —CH₂—NH—C(O)—CH₂— groups is as a C₃ alkyl chain optionally interrupted by up to two —C(O)— or —NH— moieties. Cyclic groups can also form linkers: e.g. a 1,6-cyclohexanediyl can be a linker between two R groups, as in

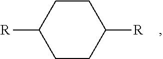

A linker can additionally be optionally substituted in any portion or position.

Divalent groups of the type R—CH═ or R₂C═, wherein both free valences are in the same atom and are attached the same substituent, are also possible. In this case, they will be referred to by their IUPAC accepted names. For instance an alkylidene (such as, for example, a methylidene (═CH₂) or a ethylidene (═CH—CH₃)) would not be encompassed by the definition of a linker in this disclosure.

The term “alkylene” denotes a straight chain or branched saturated hydrocarbon linking group containing the relevant number of carbon atoms (e.g. methylene (1 carbon), ethylene (2 carbons), propylene (3 carbons), etc.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W., Wuts, P. G in “Protective Groups in Organic Synthesis”, Third Edition, John Wiley & Sons, New York: 1999, the entire contents of which are hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

As used herein, “amide coupling agent” or “amide coupling reagent” means a compound that reacts with the hydroxyl moiety of a carboxy moiety thereby rendering it susceptible to nucleophilic attack. Exemplary amide coupling agents include DIC (diisopropylcarbodiimide), EDCI (1-Ethyl-3-(3-dimethylaminopropypcarbodiimide), DCC (dicyclohexylcarbodiimide), BOP (Benzotriazol-1-yloxy-tris(dimethylamino)-phosphonium hexafluorophosphate), pyBOP ((Benzotriazol-1-yloxy)tripyrrolidinophosphoniurn Hexafluorophosphate), etc.

The compounds of the invention are defined herein by their chemical structures and/or chemical names. Where a compound is referred to by both a chemical structure and a chemical name, and the chemical structure and chemical name conflict, the chemical structure is determinative of the compound's identity.

One embodiment of the invention is a compound represented by Structural Formula I, or a pharmaceutically acceptable salt thereof:

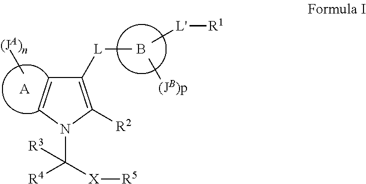

wherein:

Ring A is a 5 to 10-membered cycloaliphatic ring; or a 4 to 10-membered heterocycle; wherein said heterocycle contains from 1 to 3 ring heteroatoms independently selected from N, O or S;

n is an integer selected from 0 to 6;

each occurrence of J^{A} is independently selected from halogen, C₁₋₄ alkyl, C₁₋₄ haloalkyl, C₁₋₄ alkoxy, C₁₋₄ haloalkoxy, —CN, —OH, oxo, ═NOR⁶, —NH(C₁₋₄ alkyl), —N(C₁₋₄ alkyl)₂ or —NH₂;

R⁶ is selected from C₁₋₆ alkyl or —(C₁₋₆ alkyl)-(C₆₋₁₀ aryl);

Ring B is a monocyclic or bicyclic ring selected from phenyl or a 5 to 10-membered heteroaryl ring; wherein said heteroaryl ring contains 1 to 3 ring heteroatoms independently selected from N, O or S;

p is an integer selected from 0 to 2;

each occurrence of r is independently selected from a halogen, —NO₂, —CN, —OH, —SH, —NH₂, C₁₋₄ alkyl, C₁₋₄ haloalkyl, —O(C₁₋₄ alkyl), —O(C₁₋₄ haloalkyl), —S(C₁₋₄ alkyl), —NH(C₁₋₄ alkyl) or —N(C₁₋₄ alkyl)₂;

L is a linker selected from a methylene, —O—, —S(O)ₘ— or —NR⁹—; wherein said methylene is optionally and independently substituted with 1 or 2 instances of halogen or C₁₋₄ alkyl; wherein m is an integer selected from 0 to 2;

R⁹ is selected from hydrogen or C₁₋₄ alkyl;

L′ is a linker selected from —Y—SO₂—, —Z—C(O)—, —C(O)—Z— or a C₁₋₂ alkylene linker; wherein:

when ring B is phenyl, then Y is selected from a methylene or —NR¹⁰— and Z is selected from a single bond, a methylene or —NR¹⁰—; or

alternatively, when ring B is phenyl, L′ and R¹ together form a 5 to 6-membered heterocyclic ring which is linked to the phenyl ring B through a N ring atom; wherein said 5 to 6 membered heterocyclic ring optionally contains a heteroatom selected from N, O or S in addition to the N ring atom linked to the phenyl ring B; wherein said heteroaryl ring formed by L′ and R¹ is optionally and independently substituted by up to two instances of R⁸; or

alternatively, when ring B is a 5 to 10-membered heteroaryl ring, then Y is selected from a single bond, a methylene or —NR¹⁰— and Z is selected from a single bond, a methylene or —NR¹⁰—;

R¹⁰ is selected from hydrogen, C₁₋₆ alkyl or —(C₁₋₆ alkyl)-(C₆₋₁₀ aryl) group;

R¹ is selected from a C₁₋₆ aliphatic, C₁₋₆ haloaliphatic or a monocyclic ring selected from a 3 to 8-membered cycloaliphatic, a phenyl ring, a 5 to 6-membered heteroaryl or a 4 to 8-membered heterocycle; wherein said heteroaryl or heterocycle contains from 1 to 3 ring heteroatoms independently selected from N, O or S; and wherein when R¹ is a ring, it is optionally and independently substituted with up to three instances of R⁸;

R⁸ at each occurrence is independently selected from halogen, C₁₋₄ alkyl, C₁₋₄ haloalkyl, a 6 to 10 membered arylalkoxy group, C₁₋₄ alkoxy, —C(O)O(C₁₋₄ alkyl), —C(O)(C₁₋₄ alkyl), —CN, —OH, —NH₂, —NH(C₁₋₄ alkyl) or —N(C₁₋₄ alkyl)₂;

R² is selected from hydrogen, halogen, C₁₋₄ alkyl, C₁₋₄ haloalkyl, C₁₋₄ alkoxy, C₁₋₄ haloalkoxy, —CN or —OH;

R³ and R⁴ are each independently selected from hydrogen, deuterium, halogen, C₁₋₆ alkyl or C₁₋₆ haloalkyl; or R³ and R⁴ taken together with the atom to which they are attached form a C₃₋₇ cycloalkyl ring;

X is selected from a direct single bond or a methylene linker, wherein said methylene is optionally substituted with up to two identical or different instances of halogen, C₁₋₄ alkyl or C₁₋₄ haloalkyl;

R⁵ is selected from —C(O)OR⁷, —C(O)O—R¹¹, C(O)N(R⁷)₂, C(O)NOR⁷ or —C(O)NSR⁷;

R⁷ at each occurrence is independently selected from a hydrogen or C₁₋₄ alkyl; and

R¹¹ at each occurrence is independently is selected form a C₁₋₆ alkyl-(C₆₋₁₀ heteroaryl), a —C₁₋₆ alkyl-(C₆₋₁₀ heterocyclyl) or a —C₁₋₆ alkyl-(C₆₋₁₀ aryl) wherein each of said heteroaryl and heterocycle contains 1 to 3 ring heteroatoms independently selected from N, O or S.

In some embodiments, ring A is a 5 to 7 membered cycloaliphatic ring or a 6 membered heterocycle; wherein said heterocycle contains 1 ring heteroatom independently selected from N or O. In other embodiments, ring A is a cycloheptane ring fused to the pyrrole ring. The compounds of this embodiment are represented by Formula II:

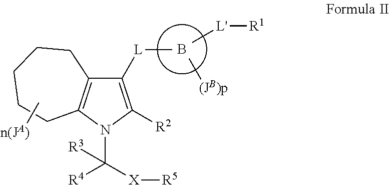

In another embodiment, ring A is a cyclopentane ring fused to the pyrrole ring and the compounds of this embodiment are represented by Formula III:

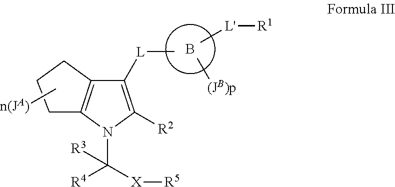

In another embodiment, ring A is a cyclohexane ring fused to the pyrrole ring and the compounds of this embodiment are represented by Formula IV:

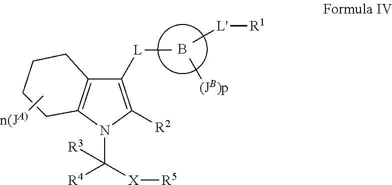

In certain embodiments of Formula I to Formula IV, two occurrences of J^{A} may each be a methyl group attached to the same ring carbon atom. In some embodiments, these compounds are represented by Formula V or Formula VI:

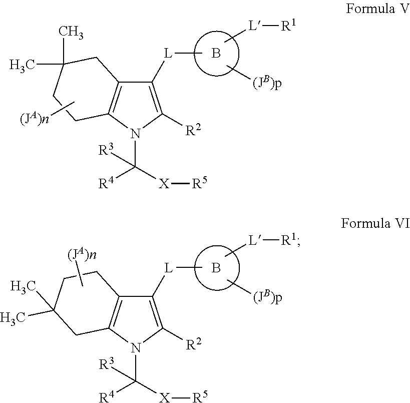

In some embodiments of Formula V and Formula VI, n is an integer selected from 0 to 4. In a particular embodiment in the compounds of Formulae V and VI, n is zero. In other embodiments, n is 1 and J^{A} is oxo, ═NO—(C₁₋₄ alkyl) or ═NO(benzyl). In still other embodiments, n is 1 and J^{A} is oxo, ═NOCH₃, ═NOCH₂CH₃, ═N—O-(t-butyl) or ═NO(benzyl).

In another embodiment, ring A is a tetrahydro-2H-pyran ring fused to the pyrrole ring and the compounds of this embodiment are represented by Formula VII:

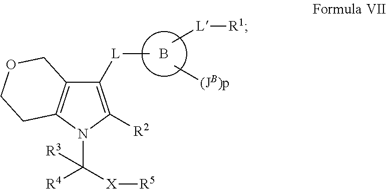

In another embodiment, ring A is a piperidin-2-one ring fused to the pyrrole ring and the compounds of this embodiment are represented by the Formula VIII:

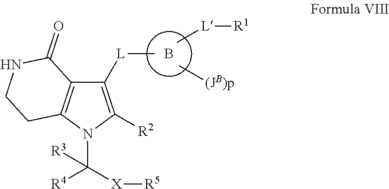

In certain embodiments of the compounds of Formulae I to VIII, ring B is ring selected from phenyl, a monocyclic 5 to 6-membered heteroaryl ring or a bicyclic 9-membered heteroaryl ring; wherein said heteroaryl ring contains up to three ring heteroatoms independently selected from N, O or S. In some embodiments of the compounds of Formulae I to VIII, ring B is selected from phenyl, thiophenyl, a monocyclic 6-membered heteroaryl ring or a bicyclic 9-membered heteroaryl ring. In other embodiments, ring B is selected from phenyl, thiophenyl, pyridinyl or indolyl. In still other embodiments, ring B is phenyl and the compounds of the invention have the Formula IX:

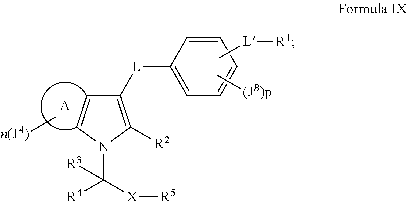

In other embodiments of the compounds of Formulae I to VIII, ring B is pyridyl and the compounds have the Formula X:

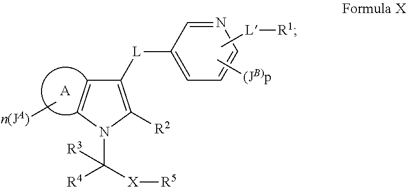

In other embodiments of the compounds of Formulae I to VIII, ring B is thiopenyl and the compounds are represented by Formula XI:

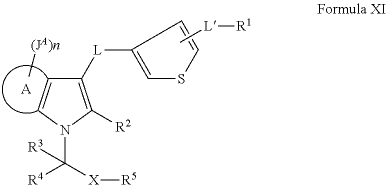

In other embodiments of the compounds of Formulae I to VIII, ring B is an indole and the compounds are represented by Formula XII:

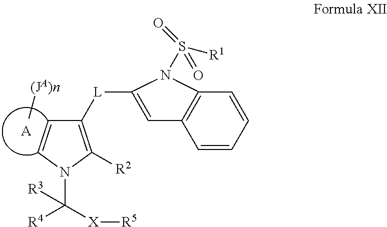

In some embodiments of the compounds of Formulae I to XII, L is selected from —CH₂—, —O—, —S—, —SO— or —SO₂—. In other embodiments L is selected from —CH₂— or —S—.

In certain embodiments of the compounds of Formula IX, L′ is —SO₂NR¹⁰ and R¹⁰ is selected from hydrogen or a C₁₋₄ alkyl. In other embodiments of the compounds of Formula IX, L′ is —SO₂— and R¹ is a heterocyclic ring selected from those depicted below:

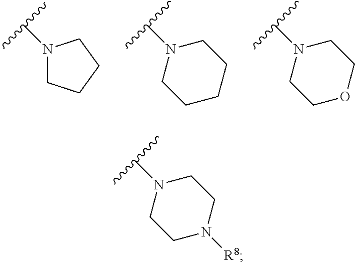

wherein L′ is attached either ortho or para to L on ring B.

In certain embodiments of the compounds of Formulae IX and X, L′ is attached ortho or para to L on ring B.

In certain embodiments of the compounds of Formulae IX to XII, R¹ is selected from a 3 to 6-membered cycloaliphatic ring, phenyl, a 6-membered heteroary or a 4 to 8-membered heterocycl; wherein said heterocycle or heteroaryl contains 1 or 2 heteroatoms selected from O or N; and wherein R¹ is substituted with up to three instances of R⁸. In other embodiments, R¹ is selected from phenyl, a cyclopropyl ring or a 5 to 6-membered heterocyclic ring; wherein said phenyl or heterocyclic ring is optionally and independently substituted with up to 3 instances of R⁸. In still other embodiments, R¹ is a phenyl ring and the compound is represented by Formula XIII; wherein s is an integer selected from 0 to 2.

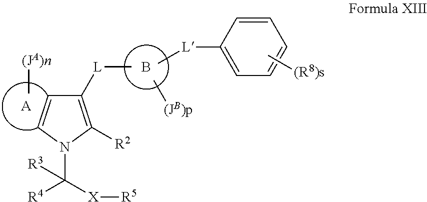

In certain embodiments of the compounds of Formula I to Formula XIII, R⁸ is chosen from C₁₋₄ alkyl, halogen, methoxy, —OH, —C(O)O(C₁₋₄ alkyl), —O(benzyl) or C₁₋₄ haloalkyl.

In certain embodiments of the compounds of Formulae I to XIII, R² is selected from hydrogen, fluoro, methyl, ethyl or trifluoromethyl. In other embodiments R² is methyl.

In certain embodiments of the compounds of Formulae I to XIII, R³ and R⁴ are taken together with the atom to which they are attached to form a cyclopropyl ring. In other embodiments R³ and R⁴ are each independently selected from hydrogen or methyl, wherein the methyl is optionally substituted with 1 to 3 instances of halogen, particularly fluoro. In other embodiments R³ and R⁴ are both hydrogen.

In certain embodiments of the compounds of Formulae I to XIII, X is selected from a direct single bond or —CH₂—. In particular embodiments X is a direct single bond.

In certain embodiments of the compounds of Formulae I to XIII, R⁵ is —C(O)OR⁷ and R⁷ is hydrogen or C₁₋₄ alkyl. In other embodiments, R⁵ is —C(O)OH, —C(O)OMe or —C(O)OEt. In still other embodiments, R⁵ is —C(O)OH. When R⁵ is —C(O)OH, the compounds of the invention may be present in the form of salts.

In some embodiments compounds of the invention are represented by Formula XIV:

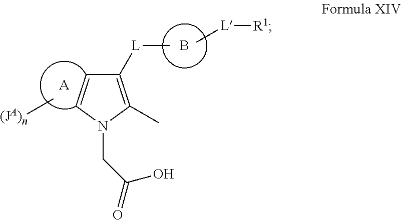

wherein:

ring A is selected from a cyclopentane ring, a cycloheptane ring, a cyclohexane ring, a tetrahydro[2H]pyran ring or a piperidine ring, each of them fused to the pyrrole ring;

J^{A} is selected independently from an oxo or methyl;

n is 0, 2 or 3;

L is —CH₂— or —S—;

ring B is chosen from a thiophenyl, a pyridinyl or an indolyl and L′ is —SO₂—, wherein L′ is ortho to L on ring B and wherein R¹ is chosen from:
(a) phenyl, optionally substituted with 0 to 2 instances of R⁸;
(b) a cyclopropyl ring;
(c) methyl;
(d) a 5 to 6-membered heterocycle containing 1 or 2 heteroatoms selected from No or O; or
alternatively,

ring B is phenyl and L′ is —SO₂— or —NR¹⁰SO₂—; wherein L′ is ortho to L on ring B; and R¹ is chosen from: N-pyrrolidinyl, N-morpholinyl, N-piperidinyl or N-piperazinyl; said N-piperazinyl optionally substituted by R⁸ on the second nitrogen ring atom; or
alternatively,

ring B is phenyl and L′ is —NR¹⁰SO₂—; wherein L′ is ortho to L on ring B; and R¹ is chosen from:
(a) phenyl, optionally substituted by 0 to 2 instances of R⁸; or
(b) a cyclopropyl ring;

and R¹⁰ is selected from hydrogen, ethyl or methyl.

In some embodiments of the compounds of Formula XIV, compounds of the invention are represented by structural Formulae XV or XVI:

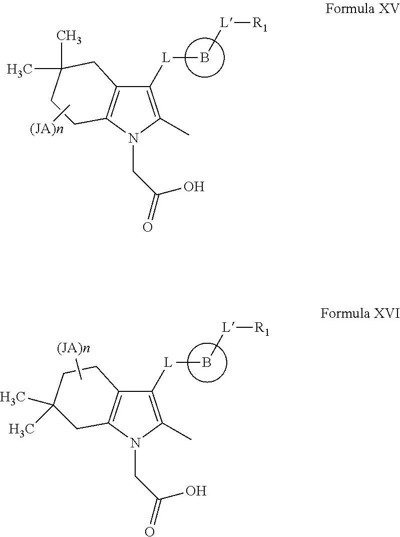

In some embodiment of the compounds of Formula XIV, XV and XVI, L′ is —SO₂— and ring B is chosen from thiophenyl, pyridyl or indolyl.

In some embodiments of the compounds of Formulae XV and XVI, n is 1, J^{A} is oxo and the compounds are represented by Formula XVII.

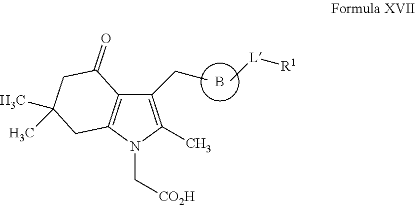

In further embodiments of Formula XVII, the compounds of the invention are represented by Formula XVIII, XIX or XX:

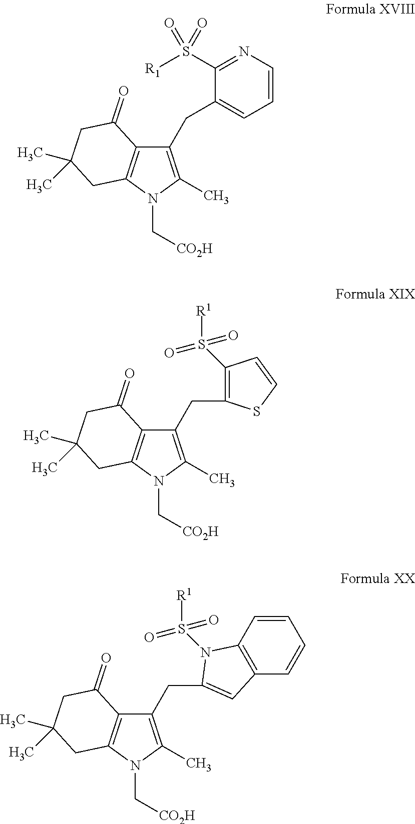

In some embodiments, the invention provides a compound represented by Formula XXI:

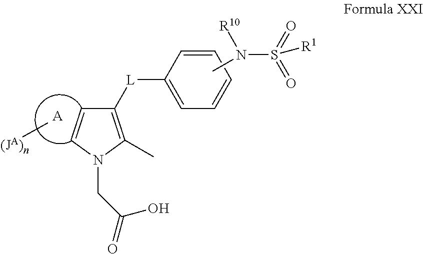

wherein:

ring A is selected from a cyclopentane ring, a cycloheptane ring, a cyclohexane ring, a tetrahydropyran ring or a piperidine ring, each of them fused to the pyrrole ring;

each J^{A} is selected from ═O, ═NO—(C₁₋₄ alkyl), ═NO-(benzyl) or methyl;

n is 0, 2 or 3;

L is —CH₂— or —S—;

R¹⁰ is selected from hydrogen, methyl, ethyl or benzyl and

R¹ is chosen from
(a) phenyl, optionally substituted by 0 to 2 instances of R⁸;
(b) a cyclopropyl ring; or
(c) N-pyrrolidinyl, N-piperidinyl, N-morpholinyl, N-piperazinyl.

In some embodiments of the compounds of Formula XXI, compounds of the invention are represented by one of Formulae XXII or XXIII:

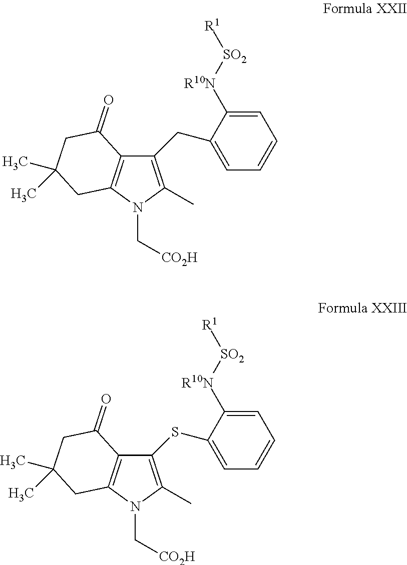

In another aspect, the invention provides a compound selected from those depicted in Table 1, or a pharmaceutically acceptable salt thereof:

TABLE 1

| No. | Compound |
|---|---|
| I-1 | 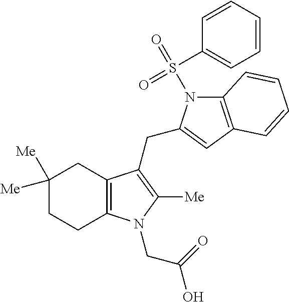 [SMILES: Cc1c(Cc2cc3ccccc3n2S(=O)(=O)c2ccccc2)c2c(n1CC(=O)O)CCC(C)(C)C2] |
| I-2 | 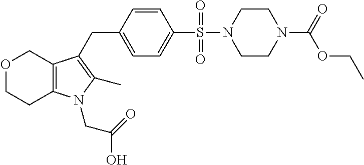 [SMILES: CCOC(=O)N1CCN(S(=O)(=O)c2ccc(Cc3c4c(n(CC(=O)O)c3C)CCOC4)cc2)CC1] |
| I-3 | 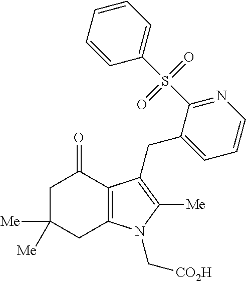 [SMILES: Cc1c(Cc2cccnc2S(=O)(=O)c2ccccc2)c2c(n1CC(=O)O)CC(C)(C)CC2=O] |
| I-4 | 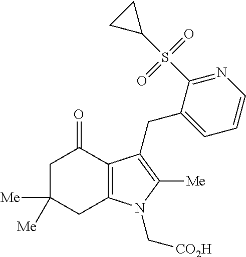 [SMILES: Cc1c(Cc2cccnc2S(=O)(=O)C2CC2)c2c(n1CC(=O)O)CC(C)(C)CC2=O] |
| I-5 | 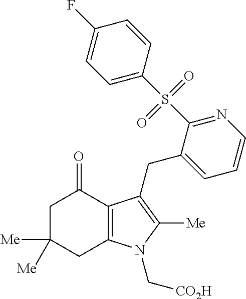 [SMILES: Cc1c(Cc2cccnc2S(=O)(=O)c2ccc(F)cc2)c2c(n1CC(=O)O)CC(C)(C)CC2=O] |
| I-6 | 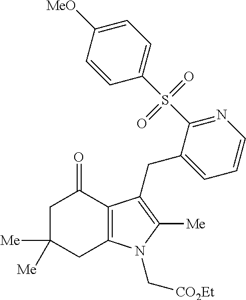 [SMILES: CCOC(=O)Cn1c(C)c(Cc2cccnc2S(=O)(=O)c2ccc(OC)cc2)c2c1CC(C)(C)CC2=O] |
| I-7 | 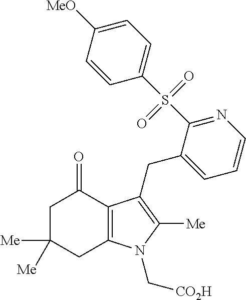 [SMILES: COc1ccc(S(=O)(=O)c2ncccc2Cc2c3c(n(CC(=O)O)c2C)CC(C)(C)CC3=O)cc1] |
| I-8 | 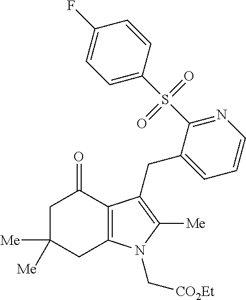 [SMILES: CCOC(=O)Cn1c(C)c(Cc2cccnc2S(=O)(=O)c2ccc(F)cc2)c2c1CC(C)(C)CC2=O] |
| I-9 | 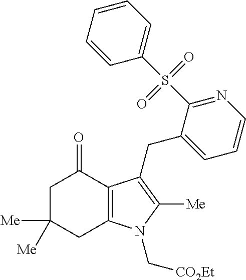 [SMILES: CCOC(=O)Cn1c(C)c(Cc2cccnc2S(=O)(=O)c2ccccc2)c2c1CC(C)(C)CC2=O] |
| I-10 | 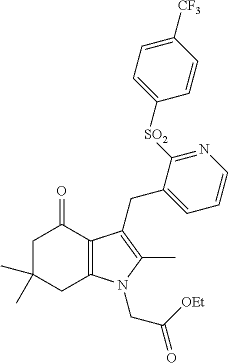 [SMILES: CCOC(=O)Cn1c(C)c(Cc2cccnc2S(=O)(=O)c2ccc(C(F)(F)F)cc2)c2c1CC(C)(C)CC2=O] |
| I-11 | 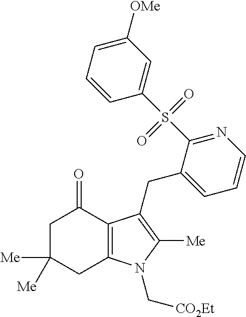 [SMILES: CCOC(=O)Cn1c(C)c(Cc2cccnc2S(=O)(=O)c2cccc(OC)c2)c2c1CC(C)(C)CC2=O] |
| I-12 | 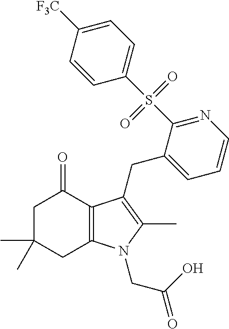 [SMILES: Cc1c(Cc2cccnc2S(=O)(=O)c2ccc(C(F)(F)F)cc2)c2c(n1CC(=O)O)CC(C)(C)CC2=O] |
| I-13 | 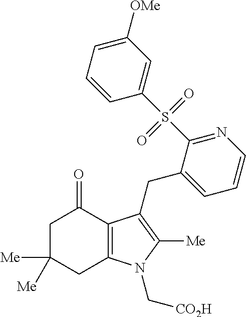 [SMILES: COc1cccc(S(=O)(=O)c2ncccc2Cc2c3c(n(CC(=O)O)c2C)CC(C)(C)CC3=O)c1] |
| I-14 | 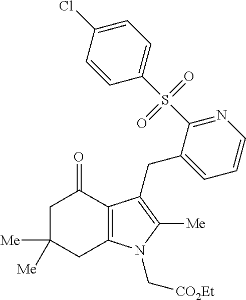 [SMILES: CCOC(=O)Cn1c(C)c(Cc2cccnc2S(=O)(=O)c2ccc(Cl)cc2)c2c1CC(C)(C)CC2=O] |
| I-15 | 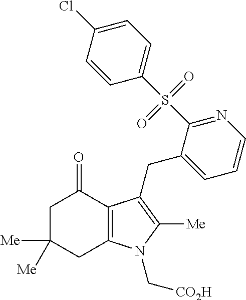 [SMILES: Cc1c(Cc2cccnc2S(=O)(=O)c2ccc(Cl)cc2)c2c(n1CC(=O)O)CC(C)(C)CC2=O] |
| I-16 | 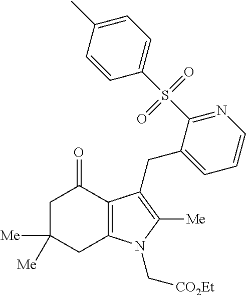 [SMILES: CCOC(=O)Cn1c(C)c(Cc2cccnc2S(=O)(=O)c2ccc(C)cc2)c2c1CC(C)(C)CC2=O] |
| I-17 | 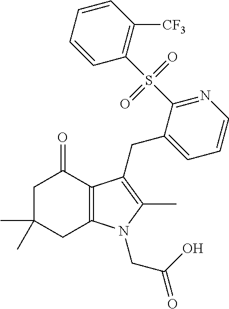 [SMILES: Cc1c(Cc2cccnc2S(=O)(=O)c2ccccc2C(F)(F)F)c2c(n1CC(=O)O)CC(C)(C)CC2=O] |
| I-18 | 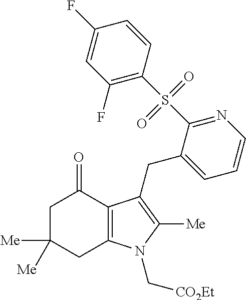 [SMILES: CCOC(=O)Cn1c(C)c(Cc2cccnc2S(=O)(=O)c2ccc(F)cc2F)c2c1CC(C)(C)CC2=O] |
| I-19 | 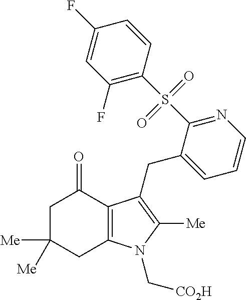 [SMILES: Cc1c(Cc2cccnc2S(=O)(=O)c2ccc(F)cc2F)c2c(n1CC(=O)O)CC(C)(C)CC2=O] |
| I-20 | 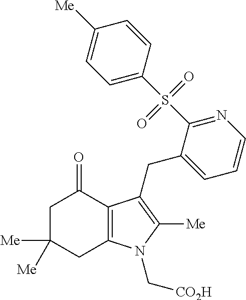 [SMILES: Cc1ccc(S(=O)(=O)c2ncccc2Cc2c3c(n(CC(=O)O)c2C)CC(C)(C)CC3=O)cc1] |
| I-21 | 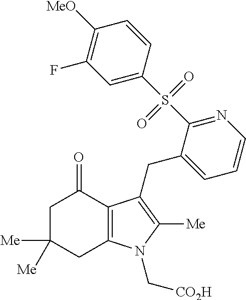 [SMILES: COc1ccc(S(=O)(=O)c2ncccc2Cc2c3c(n(CC(=O)O)c2C)CC(C)(C)CC3=O)cc1F] |
| I-22 | 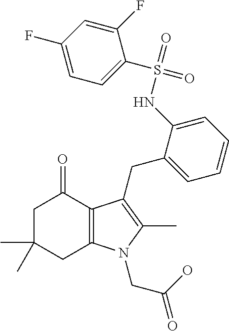 [SMILES: Cc1c(Cc2ccccc2NS(=O)(=O)c2ccc(F)cc2F)c2c(n1CC(=O)O)CC(C)(C)CC2=O] |
| I-23 | 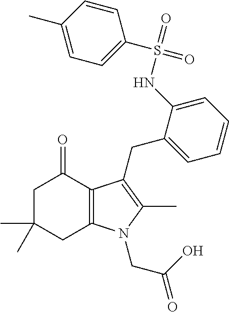 [SMILES: Cc1ccc(S(=O)(=O)Nc2ccccc2Cc2c3c(n(CC(=O)O)c2C)CC(C)(C)CC3=O)cc1] |
| I-24 | 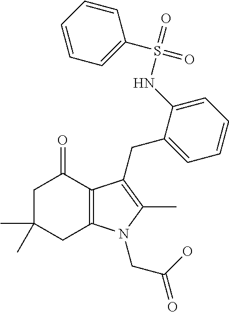 [SMILES: Cc1c(Cc2ccccc2NS(=O)(=O)c2ccccc2)c2c(n1CC(=O)O)CC(C)(C)CC2=O] |
| I-25 | 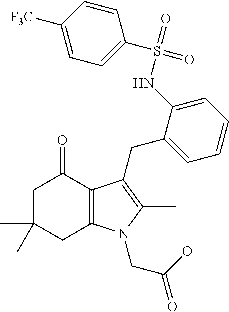 [SMILES: Cc1c(Cc2ccccc2NS(=O)(=O)c2ccc(C(F)(F)F)cc2)c2c(n1CC(=O)O)CC(C)(C)CC2=O] |
| I-26 | 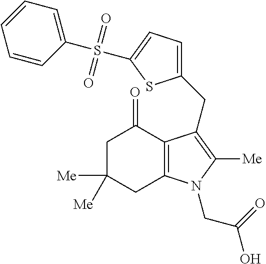 [SMILES: Cc1c(Cc2ccc(S(=O)(=O)c3ccccc3)s2)c2c(n1CC(=O)O)CC(C)(C)CC2=O] |
| I-27 | 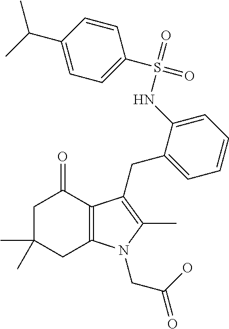 [SMILES: Cc1c(Cc2ccccc2NS(=O)(=O)c2ccc(C(C)C)cc2)c2c(n1CC(=O)O)CC(C)(C)CC2=O] |
| I-28 | 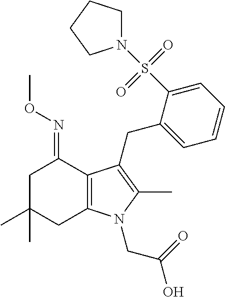 [SMILES: CO/N=C1\CC(C)(C)Cc2c1c(Cc1ccccc1S(=O)(=O)N1CCCC1)c(C)n2CC(=O)O] |
| I-29 | 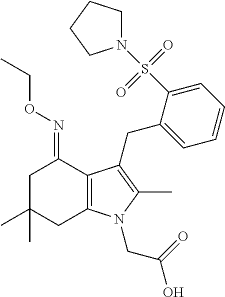 [SMILES: CCO/N=C1\CC(C)(C)Cc2c1c(Cc1ccccc1S(=O)(=O)N1CCCC1)c(C)n2CC(=O)O] |
| I-30 | 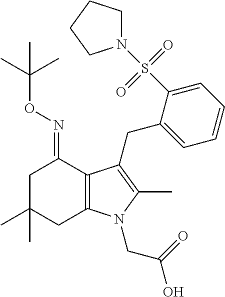 [SMILES: Cc1c(Cc2ccccc2S(=O)(=O)N2CCCC2)c2c(n1CC(=O)O)CC(C)(C)C/C2=N\OC(C)(C)C] |
| I-31 | 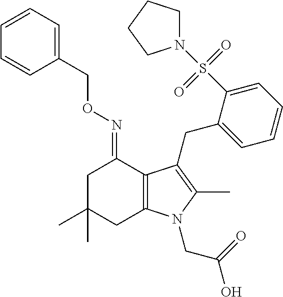 [SMILES: Cc1c(Cc2ccccc2S(=O)(=O)N2CCCC2)c2c(n1CC(=O)O)CC(C)(C)C/C2=N\OCc1ccccc1] |
| I-32 | 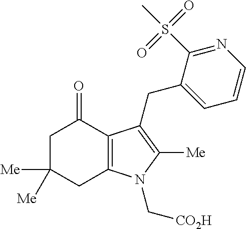 [SMILES: Cc1c(Cc2cccnc2S(C)(=O)=O)c2c(n1CC(=O)O)CC(C)(C)CC2=O] |
| I-33 | 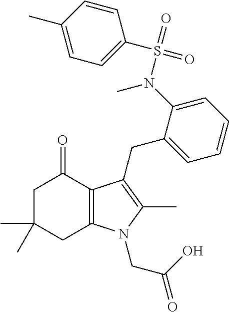 [SMILES: Cc1ccc(S(=O)(=O)N(C)c2ccccc2Cc2c3c(n(CC(=O)O)c2C)CC(C)(C)CC3=O)cc1] |
| I-34 | 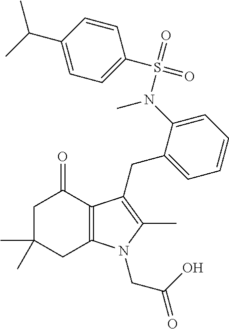 [SMILES: Cc1c(Cc2ccccc2N(C)S(=O)(=O)c2ccc(C(C)C)cc2)c2c(n1CC(=O)O)CC(C)(C)CC2=O] |
| I-35 | 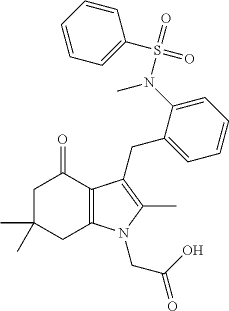 [SMILES: Cc1c(Cc2ccccc2N(C)S(=O)(=O)c2ccccc2)c2c(n1CC(=O)O)CC(C)(C)CC2=O] |
| I-36 | 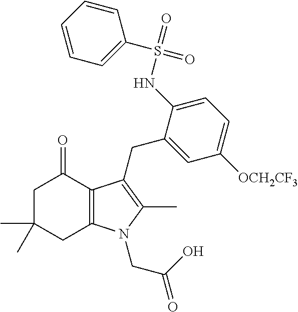 |
| I-37 | 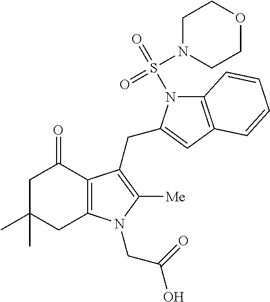 [SMILES: Cc1c(Cc2cc3ccccc3n2S(=O)(=O)N2CCOCC2)c2c(n1CC(=O)O)CC(C)(C)CC2=O] |
| I-38 | 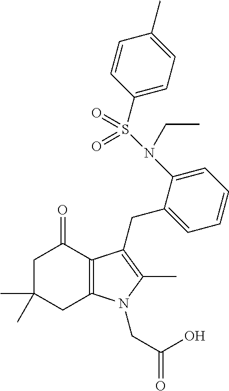 [SMILES: CCN(c1ccccc1Cc1c2c(n(CC(=O)O)c1C)CC(C)(C)CC2=O)S(=O)(=O)c1ccc(C)cc1] |
| I-39 | 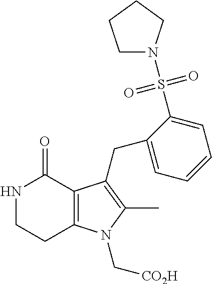 [SMILES: Cc1c(Cc2ccccc2S(=O)(=O)N2CCCC2)c2c(n1CC(=O)O)CCNC2=O] |
| I-40 | 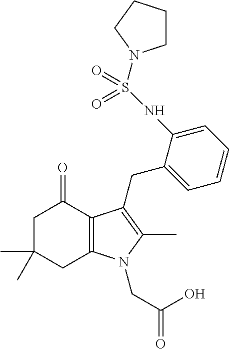 [SMILES: Cc1c(Cc2ccccc2NS(=O)(=O)N2CCCC2)c2c(n1CC(=O)O)CC(C)(C)CC2=O] |
| I-42 | 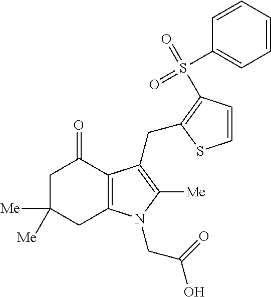 [SMILES: Cc1c(Cc2sccc2S(=O)(=O)c2ccccc2)c2c(n1CC(=O)O)CC(C)(C)CC2=O] |
| I-43 | 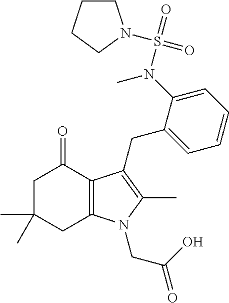 [SMILES: Cc1c(Cc2ccccc2N(C)S(=O)(=O)N2CCCC2)c2c(n1CC(=O)O)CC(C)(C)CC2=O] |
| I-44 | 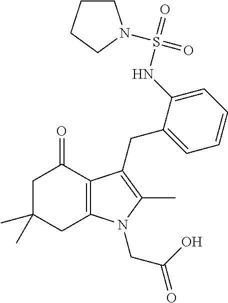 [SMILES: Cc1c(Cc2ccccc2NS(=O)(=O)N2CCCC2)c2c(n1CC(=O)O)CC(C)(C)CC2=O] |
| I-45 | 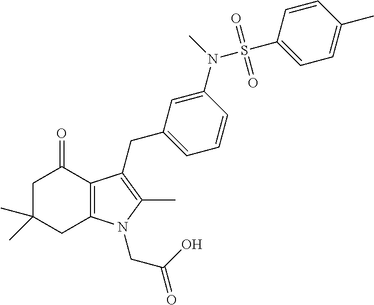 |
| I-46 | 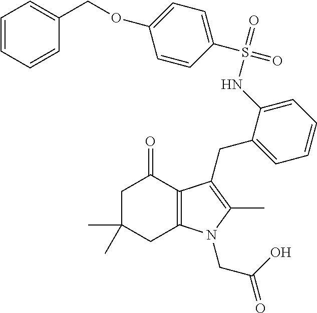 [SMILES: Cc1c(Cc2ccccc2NS(=O)(=O)c2ccc(OCc3ccccc3)cc2)c2c(n1CC(=O)O)CC(C)(C)CC2=O] |
| I-47 | 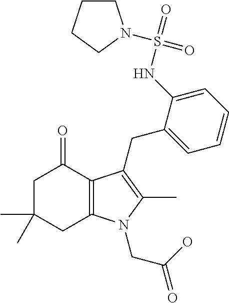 [SMILES: Cc1c(Cc2ccccc2NS(=O)(=O)N2CCCC2)c2c(n1CC(=O)O)CC(C)(C)CC2=O] |
| I-48 | 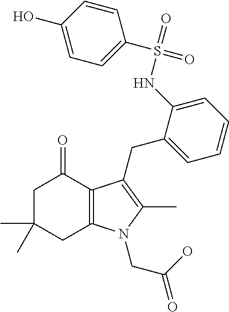 [SMILES: Cc1c(Cc2ccccc2NS(=O)(=O)c2ccc(O)cc2)c2c(n1CC(=O)O)CC(C)(C)CC2=O] |
| I-49 | 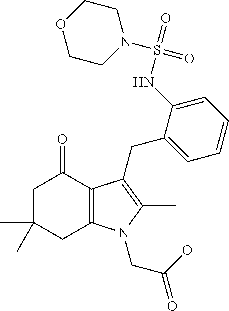 [SMILES: Cc1c(Cc2ccccc2NS(=O)(=O)N2CCOCC2)c2c(n1CC(=O)O)CC(C)(C)CC2=O] |
| I-50 | 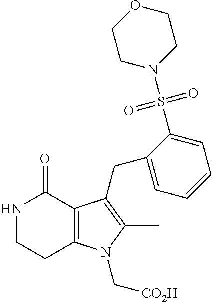 [SMILES: Cc1c(Cc2ccccc2S(=O)(=O)N2CCOCC2)c2c(n1CC(=O)O)CCNC2=O] |
| I-51 | 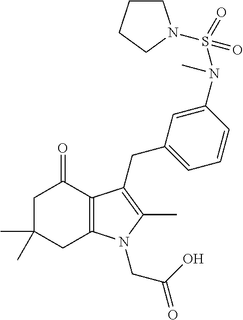 [SMILES: Cc1c(Cc2cccc(N(C)S(=O)(=O)N3CCCC3)c2)c2c(n1CC(=O)O)CC(C)(C)CC2=O] |
| I-52 | 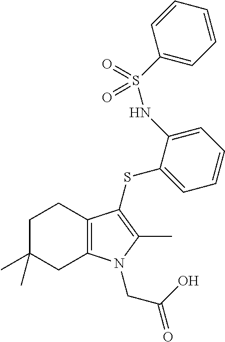 [SMILES: Cc1c(Sc2ccccc2NS(=O)(=O)c2ccccc2)c2c(n1CC(=O)O)CC(C)(C)CC2] |
| I-53 | 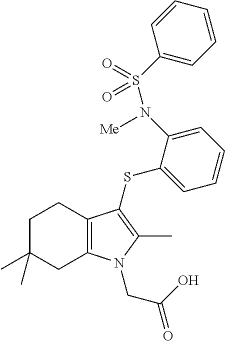 [SMILES: Cc1c(Sc2ccccc2N(C)S(=O)(=O)c2ccccc2)c2c(n1CC(=O)O)CC(C)(C)CC2] |
| I-54 | 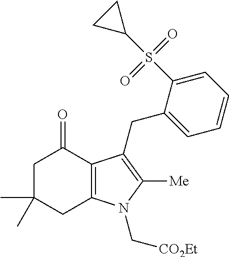 [SMILES: CCOC(=O)Cn1c(C)c(Cc2ccccc2S(=O)(=O)C2CC2)c2c1CC(C)(C)CC2=O] |
| I-55 | 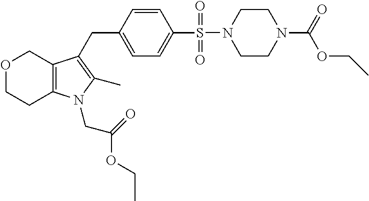 [SMILES: CCOC(=O)Cn1c(C)c(Cc2ccc(S(=O)(=O)N3CCN(C(=O)OCC)CC3)cc2)c2c1CCOC2] |
| I-56 | 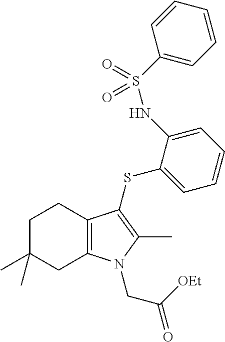 [SMILES: CCOC(=O)Cn1c(C)c(Sc2ccccc2NS(=O)(=O)c2ccccc2)c2c1CC(C)(C)CC2] |
| I-57 | 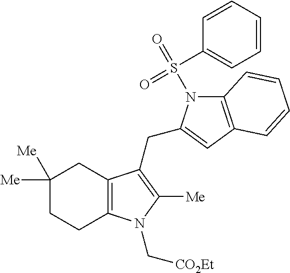 [SMILES: CCOC(=O)Cn1c(C)c(Cc2cc3ccccc3n2S(=O)(=O)c2ccccc2)c2c1CCC(C)(C)C2] |

The phrase “pharmaceutically acceptable salt,” as used herein, refers to pharmaceutically acceptable organic or inorganic salts of a compound of Formulae I to XXIII. For use in medicine, the salts of the compounds of Formulae I to XXIII will be pharmaceutically acceptable salts. Other salts may, however, be useful in the preparation of the compounds of Formulae I to XXIII or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compounds of Formulae I to XXIII are acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N.sup.1-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compounds of Formulae I to XXIII are basic or contains a sufficiently basic bioisostere, salts can be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated here by reference in its entirety.

In addition to the compounds described herein, their pharmaceutically acceptable salts may also be employed in compositions to treat or prevent the herein identified disorders.

In another aspect, the invention is a composition comprising a pharmaceutically acceptable carrier and a compound according described above.

The compounds herein disclosed, and their pharmaceutically acceptable salts, may be formulated as pharmaceutical compositions or “formulations”.

A typical formulation is prepared by mixing a compound of Formula I to XXIII, or a pharmaceutically acceptable salt thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compounds of Formula I to XXIII are being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., a compound of Formula I to XXIII or pharmaceutical composition thereof) or aid in the manufacturing of the pharmaceutical product (i.e., medicament).

The formulations may be prepared using conventional dissolution and mixing procedures. For example, the bulk drug substance (i.e., a compound of Formula I to XXIII, a pharmaceutically acceptable salt, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8. When the agent described herein is a solid amorphous dispersion formed by a solvent process, additives may be added directly to the spray-drying solution when forming the mixture such as the additive is dissolved or suspended in the solution as a slurry, which can then be spray dried. Alternatively, the additives may be added following spray-drying process to aid in the forming of the final formulated product.

The compounds of Formulae I to XXIII or a pharmaceutically acceptable salt thereof is typically formulated into pharmaceutical dosage forms to provide an easily controllable dosage of the drug and to enable patient compliance with the prescribed regimen. Pharmaceutical formulations of compounds of Formulae I to XXIII, or a pharmaceutically acceptable salt thereof, may be prepared for various routes and types of administration. Various dosage forms may exist for the same compound, since different medical conditions may warrant different routes of administration.

The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. As a general proposition, the initial pharmaceutically effective amount of the inhibitor administered will be in the range of about 0.01-100 mg/kg per dose, namely about 0.1 to 20 mg/kg of patient body weight per day, with the typical initial range of compound used being 0.3 to 15 mg/kg/day.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically or pharmaceutically effective amount” of the compound to be administered will be governed by such considerations, and is the minimum amount necessary to ameliorate, cure or treat the disease or disorder or one or more of its symptoms.

The pharmaceutical compositions of Formulae I to XXIII will be formulated, dosed, and administered in a fashion, i.e., amounts, concentrations, schedules, course, vehicles, and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners, such as the age, weight, and response of the individual patient.

The term “prophylactically effective amount” refers to an amount effective in preventing or substantially lessening the chances of acquiring a disease or disorder or in reducing the severity of the disease or disorder or one or more of its symptoms before it is acquired or before the symptoms develop. Roughly, prophylactic measures are divided between primary prophylaxis (to prevent the development of a disease) and secondary prophylaxis (whereby the disease has already developed and the patient is protected against worsening of this process).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively; in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”. In general, one can provide for controlled release of the agents described herein through the use of a wide variety of polymeric carriers and controlled release systems including erodible and non-erodible matrices, osmotic control devices, various reservoir devices, enteric coatings and multiparticulate control devices.

“Sustained-release preparations” are the most common applications of controlled release. Suitable examples of sustained-release preparations include semipermeable matrices of solid hydrophobic polymers containing the compound, which matrices are in the form of shaped articles, e.g. films, or microcapsules. Examples of sustained-release matrices include polyesters, hydrogels (for example, poly(2-hydroxyethyl-methacrylate), or poly(vinylaicohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent deaggregation to fine particles. This provides a larger surface area exposed to the dissolution medium, resulting in a faster dissolution rate.

Agents described herein can be incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use. One ingredient of this water-swollen matrix is the water-swellable, erodible, or soluble polymer, which may generally be described as an osmopolymer, hydrogel or water-swellable polymer. Such polymers may be linear, branched, or crosslinked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 daltons, for example, the Dow Methocel™ series E5, E15LV, E50LV and K100LY) and high viscosity (MW greater than 50,000 daltons, for example, E4MCR, E10MCR, K4M, K15M and K100M and the Methocel™ K series) HPMC. Other commercially available types of HPMC include the Shin Etsu Metolose 90SH series.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGITO, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

Alternatively, the agents of the present invention may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g methyl acrylate-methyl methacrylate copolymers, polyvinyl chloride, polyethylene), hydrophilic polymers (e.g. ethyl cellulose, cellulose acetate, crosslinked polyvinylpyrrolidone (also known as crospovidone)), and fatty compounds (e.g. carnauba wax, microcrystalline wax, and triglycerides). Such devices are described further in Remington: The Science and Practice of Pharmacy, 20th edition (2000).

As noted above, the agents described herein may also be incorporated into an osmotic control device. Such devices generally include a core containing one or more agents as described herein and a water-permeable, non-dissolving and non-eroding coating surrounding the core which controls the influx of water into the core from an aqueous environment of use so as to cause drug release by extrusion of some or all the core to the environment of use. In certain embodiments, the coating is polymeric, aqueous-permeable, and has at least one delivery port. The core of the osmotic device optionally includes an osmotic agent which acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head). Nonlimiting examples of osmotic control devices are disclosed in U.S. patent application Ser. No. 09/495,061.

The amount of water-swellable hydrophilic polymers present in the core may range from about 5 to about 80 wt % (including for example, 10 to 50 wt %). Non limiting examples of core materials include hydrophilic vinyl and acrylic polymers, polysaccharides such as calcium alginate, polyethylene oxide (PEO), polyethylene glycol (PEG), polypropylene glycol (PPG), poly (2-hydroxyethyl methacrylate), poly (acrylic) acid, poly (methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolate. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to thereby effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Nonlimiting examples of standard disintegrants include materials such as sodium starch glycolate (e.g., Explotab™ CLV), microcrystalline cellulose (e.g., Avicel™), microcrystalline: silicified cellulose (e.g., ProSoIv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 1-25% of the core agent.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described, for example, in EP378404.

When an agent described herein is a liquid or oil, such as a lipid vehicle formulation, for example as described in WO05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

As further noted above, the agents described herein may be provided in the form of microparticulates, generally ranging in size from about 10 μm to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art. Such multiparticulates may be made by any known process, such as wet- and dry-granulation processes, extrusion/spheronization, roller-compaction, melt-congealing, or by spray-coating seed cores. For example, in wet- and dry-granulation processes, the agent described herein and optional excipients may be granulated to form multiparticulates of the desired size.

The agents can be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The compounds described herein can be incorporated into pharmaceutically-acceptable nanoparticle, nanosphere, and nanocapsule formulations (Delie and Blanco-Prieto 2005 Molecule 10:65-80). Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, ultrafine particles (sized around 0.1 μm) can be designed using polymers able to be degraded in vivo (e.g. biodegradable polyalkyl-cyanoacrylate nanoparticles). Such particles are described in the prior art (Couvreur et al, 1980; 1988; zur Muhlen et al., 1998; Zambaux et al. 1998; Pinto-Alphandry et al., 1995 and U.S. Pat. No. 5,145,684).

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g. using capsules, powders, solutions, suspensions, tablets, sublingual tablets and the like), by inhalation (e.g. with an aerosol, gas, inhaler, nebulizer or the like), to the ear (e.g. using ear drops), topically (e.g. using creams, gels, liniments, lotions, ointments, pastes, transdermal patches, etc), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral-administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

Formulations of a compound of Formulae I to XXIII that are suitable for oral administration may be prepared as discrete units such as tablets, pills, troches, lozenges, aqueous or oil suspensions, dispersible powders or granules, emulsions, hard or soft capsules, e.g. gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g. for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending the compounds of Formulae I to XXIII in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds of Formulae I to XXIII contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds of Formulae I to XIII may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds of Formula I to XXIII include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a particle size for example in the range of 0.1 to 500 micros (including particles in a range between 0.1 and 500 microns in increments microns such as 0.5, 1, 30, 35 microns, etc) which is administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In another aspect, a compound of Formulae I to XXIII or a pharmaceutically acceptable salt thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary, compositions may be administered parenterally, orally or by any other desired route.

In another embodiment the present invention also provides a method for preventing or lessening the severity of or treating a patient suffering from a disease or disorder involving the CRTH2 receptor in a patient, said method comprising administering to said patient a therapeutically or prophylactically effective amount of a compound of the invention to said patient.

In some embodiments, the disease or disorder being treated is asthma, atopic dermatitis, allergic rhinitis, allergy, Grave's Disease, acute rhinitis, hatrophic rhinitis or chronic rhinitis, rhinitis caseosa, hypertrophic rhinitis, rhinitis purulenta, rhinitis sicca, rhinitis medicamentosa, membranous rhinitis, croupous rhinitis, fibrinous rhinitis, pseudomembranous rhinitis, scrofulous rhinitis, perennial allergic rhinitis, seasonal rhinitis, rhinitis nervosa, vasomotor rhinitis, antitussive activity, bronchial asthma, allergic asthma, intrinsic asthma, extrinsic asthma, dust asthma, chronic asthma, inveterate asthma, late asthma, airway hyper-responsiveness, bronchitis, chronic bronchitis, eosinophilic bronchitis, chronic inflammatory diseases of the lung which result in interstitial fibrosis, interstitial lung diseases (ILD), idiopathic pulmonary fibrosis, ILD associated with rheumatoid arthritis, scleroderma lung disease, chronic obstructive pulmonary disease (COPD), chronic sinusitis, conjunctivitis, allergic conjunctivitis, cystic fibrosis, fanner's lung, fibroid lung, hypersensitivity lung disease, hypersensitivity pneumonitis, idiopathic interstitial pneumonia, nasal congestion, nasal polyposis, otitis media, chronic cough associated with inflammation, systemic anaphylaxis, hypersensitivity responses, drug allergies, insect sting allergies, food related allergies, food-related allergies with symptoms of migraine, rhinitis or eczema, arthritis, rheumatic arthritis, infectious arthritis, autoimmune arthritis, seronegative arthritis, spondyloarthropathy, ankylosing spondylitis, psoriatic arthritis, Reiter's disease, osteoarthritis, systemic sclerosis, psoriasis, atopical dermatitis, contact dermatitis, seborrheic dermatitis, cutaneous eosinophilias, chronic skin ulcers, cutaneous lupus erythematosus, contact hypersensitivity, allergic contact dermatitis, eosinophilic folliculitis, Coeliac disease, cholecystitis, Crohn's disease, enteritis, eosinophilic gastroenteritis, eosinophilic esophagitis, enteropathy associated with seronegative arthropathies, gastritis, inflammatory bowel disease, irritable bowel disease, acute and chronic allograft rejection following solid organ transplant, chronic graft versus host disease, skin graft rejection, bone marrow transplant rejection, inflammation, hyperalgesia, allodynia, neuropathic pain, lupus erythematosus; systemic lupus, erythematosus; Hashimoto's thyroiditis, Grave's disease, type I diabetes, eosinophilia fasciitis, hyper IgE syndrome, idiopathic thrombocytopenia pupura; post-operative adhesions, ischemic/reperfusion injury in the heart, brain, peripheral limb hepatitis, mastocytosis, mastitis, vaginitis, vasculitis, myositis, basophilic leukemia, basophilic leukocytosis, or Churg-Strauss syndrome. More preferably the disease or disorder being treated with a composition of the invention is asthma or preventing an asthma attack. The disease or disorder being treated with a composition of the invention is may also be allergic rhinitis. The disease or disorder being treated may also be Chronic Obstructive Pulmonary Disease. The disease or disorder being treated may also be neuropathic pain. The disease or disorder being treated may also be atopic dermatitis. The disease or disorder being treated may also be allergic conjunctivitis. The disease or disorder being treated may also be gastrointestinal tract related diseases and disorders selected from Crohn's disease, eosinophilic gastroenteritis, eosinophilic esophagitis, inflammatory bowel disease or irritable bowel disease.

In some embodiments, compounds of the invention are CRTH2 antagonists that can be used, for example, to prevent and/or treat conditions or disorders in which it is considered desirable to reduce or eliminate CRTH2 activity. CRTH2 antagonists may be used to aid in preventing and/or treating a disease or disorder mediated, regulated or influenced by, for example, Th2 cells, eosinophils, basophils, platelets, Langerhans cells, dendritic cells or mast cells. They also may be used to aid in the prevention or treatment of a disease or disorder mediated, regulated or influenced by PGD₂ and metabolites thereof, such as 13,14-dihydro-15-keto-PGD2 and 15-deoxy-Al 2,1′-PGD₂.

The terms, “disease”, “disorder”, and “condition” may be used interchangeably here to refer to a CRTH2 receptor mediated medical or pathological condition.

As used herein, the terms “subject” and “patient” are used interchangeably. The terms “subject” and “patient” refer to an animal (e.g., a bird such as a chicken, quail or turkey, or a mammal). A “mammal” includes a non-primate (e.g., a cow, pig, horse, sheep, rabbit, guinea pig, rat, cat, dog, and mouse) and a primate (e.g., a monkey, chimpanzee and a human), and in particular a human. In one embodiment, the subject is a non-human animal such as a farm animal (e.g., a horse, cow, pig or sheep), or a pet (e.g., a dog, cat, guinea pig or rabbit). In another embodiment, the subject is a human.

The term “biological sample”, as used herein, refers to an in vitro or ex vivo sample, and includes, without limitation, cell cultures or extracts thereof; biopsied material obtained from a mammal or extracts thereof; blood, saliva, urine, faeces, semen, tears, lymphatic fluid, ocular fluid, vitreous humour, or other body fluids or extracts thereof.

“Treat”, “treating” or “treatment” with regard to a disorder or disease refers to alleviating or abrogating the cause and/or the effects of the disorder or disease. As used herein, the terms “treat”, “treatment” and “treating” refer to the reduction or amelioration of the progression, severity and/or duration of a CRTH2 receptor mediated condition, or the amelioration of one or more symptoms (preferably, one or more discernible symptoms) of said condition, resulting from the administration of one or more therapies (e.g., one or more therapeutic agents such as a compound or composition of the invention). In specific embodiments, the terms “treat”, “treatment” and “treating” refer to the amelioration of at least one measurable physical parameter of a CRTH2 receptor mediated condition. In other embodiments the terms “treat”, “treatment” and “treating” refer to the inhibition of the progression of a CRTH2 receptor mediated condition, either physically by, e.g., stabilization of a discernible symptom, physiologically by, e.g., stabilization of a physical parameter, or both.

The term “preventing” as used herein refers to administering a medicament beforehand to forestall or obtund an attack. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended. For example, in the Physician's Desk Reference, a standard text in the field, the term “prevent” occurs hundreds of times. As used herein, the terms “prevent”, “preventing” and “prevention” with regard to a disorder or disease refer to averting the cause and/or effects of a disease or disorder prior to the disease or disorder manifesting itself. The terms “prophylaxis” or “prophylactic use”, as used herein, refer to any medical or public health procedure whose purpose is to prevent, rather than treat or cure a disease. As used herein, the terms “prevent”, “prevention” and “preventing” also refer to the reduction in the risk of acquiring or developing a given condition, or the reduction or inhibition of the recurrence or said condition in a subject who is not ill, but who has been or may be near a person with the disease.

In one embodiment, the methods of the invention are a preventative or “pre-emptive” measure to a patient, preferably a human; having a predisposition to developing a CRTH2 receptor related disease or symptom. For example, the compounds described herein may be used to prevent the onset or re-occurrence of an asthma attack or allergic rhinitis, or prevent the onset or re-occurrence of atopic dermatitis.

The compounds and pharmaceutical compositions described herein can be used alone or in combination therapy for the treatment or prevention of a disease or disorder mediated, regulated or influenced by, for example, Th2 cells, eosinophils, basophils, platelets, Langerhans cells, dendritic cells or mast cells. They also may be used to aid in the prevention or treatment of a disease or disorder mediated, regulated or influenced by PGD₂ and metabolites thereof, such as 13,14-dihydro-15-keto-PGD₂ and 15-deoxy-Al 2,1′-PGD₂.

CRTH2 antagonists may be useful in the prevention and/or treatment of disease and disorders characterized by undesirable activation of Th2 cells, eosinophils, and basophils e.g., asthma, atopic dermatitis, allergic rhinitis, allergies (e.g., food allergies, dust allergies, pollen allergies, mold allergies), and Grave's Disease. CRTH2 antagonists or agonists may be used to aid in preventing and/or treating the following types of diseases, conditions and disorders:

(1) respiratory tract/obstructive airways diseases and disorders including: acute-, allergic, hatrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), antitussive activity, asthma (such as bronchial, allergic, intrinsic, extrinsic and dust asthma particularly chronic or inveterate asthma (e.g. late asthma and airways hyper-responsiveness)), bronchitis (including chronic and eosinophilic bronchitis), chronic inflammatory diseases of the lung which result in interstitial fibrosis, such as interstitial lung diseases (ILD) (e.g., idiopathic pulmonary fibrosis, or ILD associated with rheumatoid arthritis, scleroderma lung disease, or other autoimmune conditions), chronic obstructive pulmonary disease (COPD) (such as irreversible COPD), chronic sinusitis, conjunctivitis (e.g. allergic conjunctivitis), cystic fibrosis, fanner's lung and related diseases, fibroid lung, hypersensitivity lung diseases, hypersensitivity pneumonitis, idiopathic interstitial pneumonia, nasal congestion, nasal polyposis, otitis media, and chronic cough associated with inflammation or iatrogenic induced;

(2) systemic anaphylaxis or hypersensitivity responses, drug allergies (e.g., to penicillin, cephalosporins), insect sting allergies, and food related allergies which may have effects remote from the gut (such as migraine, rhinitis and eczema);

(3) bone and joint related diseases and disorders including: arthritis including rheumatic, infectious, autoimmune, seronegative, spondyloarthropathies (such as ankylosing spondylitis, psoriatic arthritis and Reiter's disease), osteoarthritis, and systemic sclerosis;

(4) skin and eye related diseases and disorders including: psoriasis, atopical dermatitis, contact dermatitis, other eczmatous, dermitides, seborrheic dermatitis, cutaneous eosinophilias, chronic skin ulcers, cutaneous lupus erythematosus, contact hypersensitivity/allergic contact dermatits (including sensitivity to poison ivy, sumac, or oak), and eosinophilic folliculitis (Ofuji's disease);

(5) gastrointestinal tract related diseases and disorders including: Coeliac disease, cholecystitis, Crohn's disease, enteritis (including eosinophilic gastroenteritis), eosinophilic esophagitis, enteropathy associated with seronegative arthropathies, gastritis, inflammatory bowel disease and irritable bowel disease;

(6) transplant rejection related conditions including: acute and chronic allograft rejection following solid organ transplant, for example, transplantation of kidney, heart, liver, lung, and cornea, chronic graft versus host disease, skin graft rejection, and bone marrow transplant rejection;

(7) inflammation;

(8) hyperalgesia, allodynia and neuropathic pain; and

(9) other diseases and disorders including: lupus erythematosus; systemic lupus, erythematosus; Hashimoto's thyroiditis, Grave's disease, type I diabetes, eosinophilia fasciitis, hyper IgE syndrome, idiopathic thrombocytopenia pupura; post-operative adhesions, ischemic/reperfusion injury in the heart, brain, peripheral limbs hepatitis (alcoholic, steatohepatitis and chronic viral), mastocytosis (cutaneous and systemic), mastitis (mammary gland), vaginitis, vasculitis (e.g., necrotizing, cutaneous, and hypersensitivity vasculitis), myositis (including polyinyositis, derinatomyositis), basophil related diseases including basophilic leukemia and basophilic leukocytosis, and eosinophil related diseases such as Churg-Strauss syndrome.

Compounds and compositions of the invention are also useful for veterinary treatment of companion animals, exotic animals and farm animals, including, without limitation; dogs, cats, mice, rats, hamsters, gerbils, guinea pigs, rabbits, horses, pigs and cattle.

In another embodiment, the invention provides a method of reducing CRTH2 receptor activity in a biological sample, comprising contacting said biological sample with a compound or composition of the invention. Use of a CRTH2 receptor antagonist in a biological sample is useful for a variety of purposes known to one of skill in the art. Examples of such purposes include, without limitation, biological assays and biological specimen storage.

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When co-administered with other agents, e.g., when co-administered with another pain medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

When “combination therapy” is employed, an effective amount can be achieved using a first amount of a compound of Formulae I to XXIII or a pharmaceutically acceptable salt thereof and a second amount of an additional suitable therapeutic agent (e.g. an agent to treat pain).

In one embodiment of the invention, the compounds of Formulae I to XXIII and the additional therapeutic agent are each administered in an effective amount (i.e., each in an amount which would be therapeutically effective if administered alone). In another embodiment, the compound of Structural Formulae I to XXIII and the additional therapeutic agent are each administered in an amount which alone does not provide a therapeutic effect (a sub-therapeutic dose). In yet another embodiment, the compound of Structural Formulae I to XXIII can be administered in an effective amount, while the additional therapeutic agent is administered in a sub-therapeutic dose. In still another embodiment, the compound of Structural Formula I to XXIII can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When co-administration involves the separate administration of the first amount of a compound of Structural Formulae I to XXIII and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of Formulae I to XXIII and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a Second therapy (e.g., a prophylactic or therapeutic agent such as an anti-cancer agent) to a subject.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

Examples of other therapeutic agents that may be combined with a compound of the invention, either administered separately or in the same pharmaceutical compositions, include, but are not limited to:

(1) inactivating antibodies (e.g., monoclonal or polyclonal) to interleukins (e.g., IL-4 and IL-5 (for example see Leckie et al. 2000 Lancet 356:2144));

(2) soluble chemokine receptors (e.g. recombinant soluble IL-4 receptor (Steinke and Borish 2001 Respiratory Research 2:66));

(3) chemokine receptor modulators including but not limited to antagonists of CCR1 (e.g., CP-481,715 (Gladue et al. J Biol Chem 278:40473)), CCR3 (e.g., UCB35625 (Sabroe et al. J Biol Chem 2000 275:25985), CCR5 and those described in: WO0039125A1, WO02070523A1, WO03035627A1, WO03084954A1, WO04011443A1, WO04014875A1, WO04018425A1, WO04018435A1, WO04026835A1, WO04026880A1, WO04039376A1, WO04039377A1, WO04039787A1, WO04056773A1, WO04056808A1, and WO04056809A1;

(4) histamine HI receptor antagonists/antihistamines (i.e. any compound that is capable of blocking, inhibiting, reducing or otherwise interrupting the interaction between histamine and its receptor) including but not limited to:—4 astemizole, acrivastine, antazoline, asternizole, azatadine, azelastine, bromopheniramine, carbinoxamine, carebastine, cetirizine, chlorpheniramine, clemastine, cyclizine, cyproheptadine, descarboethoxyloratadine, dexchlorpheniramine, dimethindene, diphenhydramine, diphenylpyraline, doxylamine, ebastine, efletirizine, epinastine, fexofenadine, hydroxyzine, hydroxyzine, ketotifen, levocabastine, levocetirizine, levocetirizine, loratadine, meclizine, mequitazine, methdilazine, mianserin, mizolastine, noberastine, norasternizole, noraztemizole, pheniramine, picumast, promethazine, pyrilamine, temelastine, terfenadine, trimeprazine, tripelenamine, and triprolidin; leukotriene D4 receptor antagonists/leukotriene antagonists/LTD4 antagonists (i.e., any compound that is capable of blocking, inhibiting, reducing or otherwise interrupting the interaction between leukotrienes and the Cys LTI receptor) including but not limited to: zafirlukast, montelukast, montelukast sodium (Singulair®), pranlukast, iralukast, pobilukast, SKB-106,203 and compounds described as having LTD4 antagonizing activity described in U.S. Pat. No. 5,565,473;

(5) PGD2 receptor antagonists including, but not limited to, compounds described as having PGD2 antagonizing activity in United States Published Applications US20020022218, US20010051624, and US20030055077, PCT Published Applications WO9700853, WO9825919, WO03066046, WO03066047, WO03101961, WO03101981, WO04007451, WO0178697, WO04032848, WO03097042, WO03097598, WO03022814, WO03022813, and WO04058164, European Patent Applications EP945450 and EP944614, and those listed in: Torisu et al. 2004 Bioorg Med Chem Lett 14:4557, Torisu et al. 2004 Bioorg Med Chem Lett 2004 14:4891, and Torisu et al. 2004 Bioorg & Med Chem 2004 12:4685;

(6) VLA-4 antagonists;

(7) corticosteroids, such as beclomethasone, methylprednisolone, betamethasone, prednisone, prenisolone, triamcinolone, dexamethasone, fluticasone, flunisolide and hydrocortisone, and corticosteroid analogs such as budesonide;

(8) immunosuppressants such as cyclosporine (cyclosporine A, Sandimmune® Neoral®), tacrolimus (FK-506, Prograf®), rapamycin (sirolimus, Rapamune®) and other FK-506 type immunosuppressants, and mycophenolate, e.g., mycophenolate mofetil (CellCept®);

(9) non-steroidal anti-asthmatics such as β2-agonists (e.g., terbutaline, metaproterenol, fenoterol, isoetharine, albuterol, salmeterol, bitolterol and pirbuterol) and β2-agonist-corticosteroid combinations (e.g., salmeterol-fluticasone (Advair®), formoterol-budesonid (Symbicort®)), theophylline, cromolyn, cromolyn sodium, nedocromil, atropine, ipratropium, ipratropium bromide, leukotriene biosynthesis inhibitors (zileuton, BAY1005);

(10) non-steroidal antiinflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin; alclofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone);

(11) cyclooxygenase-2 (COX-2) inhibitors such as celecoxib (Celebrex®), rofecoxib (Vioxx®), valdecoxib, etoricoxib, parecoxib, nimesulide and lumiracoxib;

(12) inhibitors of phosphodiesterase type IV (PDE-IV);

(13) opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine;

(14) antithrombotic agents, such as thrombolytic agents (e.g., streptokinase, alteplase, anistreplase and reteplase), heparin, hirudin and warfarin derivatives, n-blockers (e.g., atenolol), β-adrenergic agonists (e.g., isoproterenol), ACE inhibitors and vasodilators (e.g., sodium nitroprusside, nicardipine hydrochloride, nitroglycerin and enaloprilat);

(15) anti-diabetic agents such as insulin and insulin mimetics, sulfonylureas, (e.g., glyburide, meglinatide), biguanides, e.g., metformin (Glucophage®), α-glucosidase inhibitors (acarbose), thiazolidinone compounds, e.g., rosiglitazone (Avandia®), troglitazone (Rezulin®), ciglitazone, pioglitazone (Actos®) and englitazone;

(16) preparations of interferon beta (interferon β-I α, interferon β-I β);

(17) gold compounds such as auranofin and aurothioglucose;

(18) TNF inhibitors, e.g., etanercept (Enbrel®), antibody therapies such as orthoclone (OKT3), daclizumab (Zenapax®), basiliximab (Simulec®)), infliximab (Remicade®) and D2E6 TNF antibody;

(19) lubricants or emollients such as petrolatum and lanolin, keratolytic agents, vitamin D₃ derivatives (e.g., calcipotriene and calcipotriol (Dovonex®)), PUVA, anthralin (Drithrocreme®), etretinate (Tegison®) and isotretinoin;

(20) multiple sclerosis therapeutic agents such as interferon β-I β (Betaseron®), interferon β-I α (Avonex®), azathioprine (Imurek®, Imuran®), glatirarner acetate (Capoxone®), a glucocorticoid (e.g., prednisolone) and cyclophosphamide; and

(21) other compounds such as 5-aminosalicylic acid and prodrugs thereof, DNA-alkylating agents (e.g., cyclophosphamide, ifosfamide), antimetabolites (e.g., azathioprine, 6-mercaptopurine, methotrexate, a folate antagonist, and 5-fluorouracil, a pyrimidine antagonist), microtubule disruptors (e.g., vincristine, vinblastine, paclitaxel, colchicine, nocodazole and vinorelbine), DNA intercalators (e.g., doxorubicin, daunomycin and cisplatin), DNA synthesis inhibitors such as hydroxyurea, DNA cross-linking agents, e.g., mitomycin C, hormone therapy (e.g., tamoxifen, and flutamide), and cytostatic agents, e.g., imatinib (STI571, Gleevec®) and rituxirnab (Rituxan®).

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for cotherapy use, a pharmaceutically acceptable carrier, vehicle or diluent. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of Formulae I to XXIII may be prepared according to the schemes and examples depicted and described below. Unless otherwise specified, the starting materials and various intermediates may be obtained from commercial sources, prepared from commercially available compounds or prepared using well-known synthetic methods.

### EXAMPLES

All references provided in the Examples are herein incorporated by reference. As used herein, all abbreviations, symbols and conventions are consistent with those used in the contemporary scientific literature. See, e.g. Janet S. Dodd, ed., The ACS Style Guide: A Manual for Authors and Editors, 2″ Ed., Washington, D.C.: American Chemical Society, 1997, herein incorporated in its entirety by reference.

### Example 1

### Preparation of Common Intermediate 3-((2-bromopyridin-3-ethyl)-2,6,6-trimethyl-6,7-dihydro-1H-indol-4(5H)-one

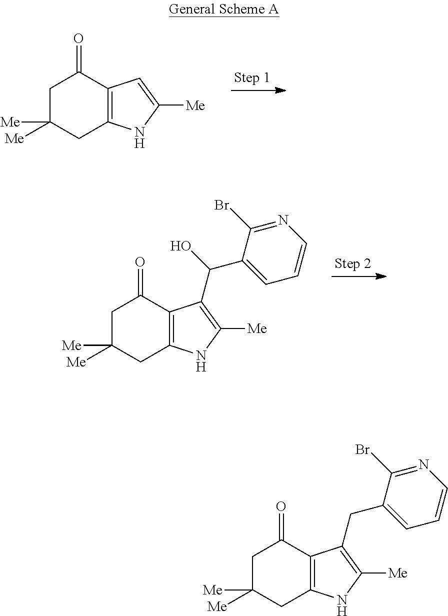 [SMILES: Cc1cc2c([nH]1)CC(C)(C)CC2=O ; Cc1[nH]c2c(c1C(O)c1cccnc1Br)C(=O)CC(C)(C)C2 ; Cc1[nH]c2c(c1Cc1cccnc1Br)C(=O)CC(C)(C)C2]

### Step 1

### Preparation of 3-((2-bromopyridin-3-yl)(hydroxy)methyl)-2,6,6-trimethyl-6,7-dihydro-1H-indol-4(5H)-one

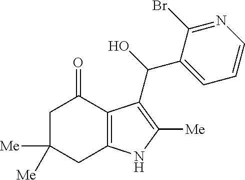 [SMILES: Cc1[nH]c2c(c1C(O)c1cccnc1Br)C(=O)CC(C)(C)C2]

To a solution of 2,6,6-trimethyl-6,7-dihydro-1H-indol-4(5H)-one (100 mg, 0.564 mmol) and 2-bromonicotinaldehyde (105 mg, 0.564 mmol) in MeOH (2.3 ml) was added a 1M aqueous solution of sodium hydroxide (1.69 ml, 1.690 mmol). The reaction was heated to 60° C. After 1 hour, the LCMS indicated the presence of a new species that is consistent with the desired product mass. After 12 hours, water was added and the reaction was worked up by the addition of saturated ammonium chloride, extracted with dichloromethane (3×50 mL), dried (sodium sulfate), filtered and concentrated to a residue which was purified on silica gel (Luknova 40 g, 20 mL/min) chromatography using 1 to 7% of 7:1 MeCN:MeOH in dichloromethane over 70 minutes affording 34(2-bromopyridin-3-yl)(hydroxy)methyl)-2,6,6-trimethyl-6,7-dihydro-1H-indol-4(5H)-one (29.0 mg, 0.0800 mmol, 14% yield) ¹H NMR (400 MHz, CDCl₃) δ (ppm): 8.22 (dd, 1H, J=4.8 Hz, J=2.0 Hz), 8.10 (br s, 1H), 7.80 (dd, 1H, J=7.6 Hz, J=2.0 Hz), 7.19 (dd, 1H, J=7.6 Hz, J=4.8 Hz), 6.81 (d, 1H, J=10.0 Hz), 6.02 (d, 1H, J=10.0 Hz), 2.66 (d, 1H, J=16.0 Hz), 2.62 (d, 1H, J=16.0 Hz), 2.46 (J=1H, J=16.4 Hz), 2.40 (d, 1H, J=16.4 Hz), 2.11 (s, 3H), 1.15 (s, 3H), 1.13 (s, 3H).

### Step 2

### Preparation of 3-((2-bromopyridin-3-yl)methyl)-2,6,6-trimethyl-6,7-dihydro-M-indol-4(5H)-one

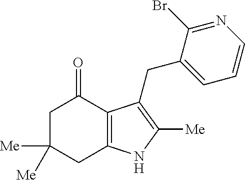 [SMILES: Cc1[nH]c2c(c1Cc1cccnc1Br)C(=O)CC(C)(C)C2]

To a solution of 34(2-bromopyridin-3-yl)(hydroxy)methyl)-2,6,6-trimethyl-6,7-dihydro-1H-indol-4(5H)-one (29.0 mg, 0.0800 mmol) in dichloromethane (1.6 mL) was added trifluoroacetic acid (67.7 μL, 0.878 mmol) followed by neat triethylsilane (25.5 μL, 0.160 mmol). The reaction was stirred at 75° C. for 30 minutes, after which it was cooled to room temperature and quenched by the addition of saturated sodium bicarbonate solution, extracted with dichloromethane (3×30 mL), dried (sodium sulfate), filtered, and concentrated to an off-white solid. This material was used in the next step without purification. ¹H NMR (400 MHz, CDCl₃) δ (ppm): 8.14 (dd, 1H, J=4.8 Hz, J=2.0 Hz), 7.88 (br s, 1H), 7.33 (dd, 1H, J=4.8 Hz, J=2.0 Hz), 7.08 (d, 1H, J=7.6 Hz, J=4.8 Hz), 4.13 (s, 2H), 2.63 (s, 2H), 2.30 (s, 2H), 2.11 (s, 3H), 1.11 (s, 6H).

### Example 2

### Synthesis of Pyridyl Analogs

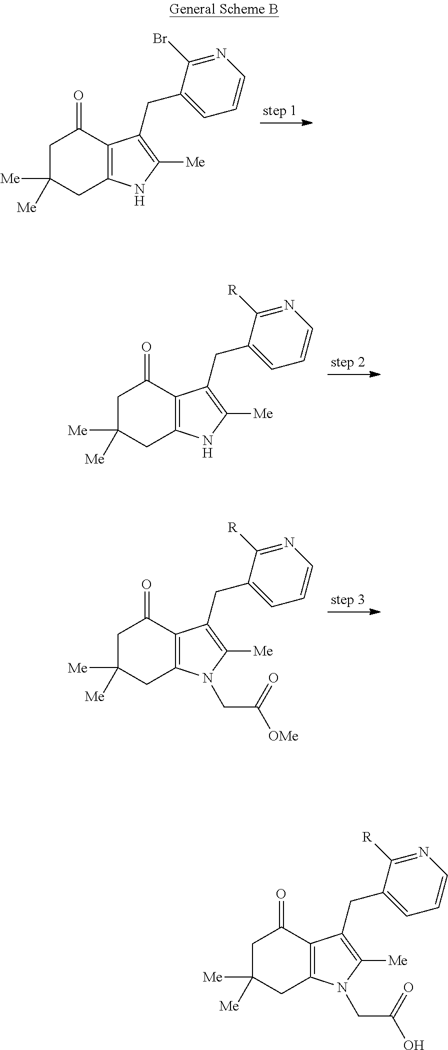

### Preparation of 2-(2,6,6-trimethyl-4-oxo-3-((2-(phenylsulfonyl)pyridin-3-yl)methyl)-4,5,6,7-tetrahydro-1H-indol-1-yl)acetic acid (I-3)

### Step 1

### Preparation of 2,6,6-trimethyl-3-((2-(phenylsulfonyl)pyridin-3-yl)methyl)-6,7-dihydro-1H-indol-4(5H)-one

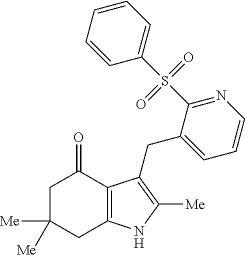 [SMILES: Cc1[nH]c2c(c1Cc1cccnc1S(=O)(=O)c1ccccc1)C(=O)CC(C)(C)C2]

To a microwave vial was added sodium benzenesulfinate (35.6 mg, 0.217 mmol) and DMSO (0.9 ml). Solid 3-((2-bromopyridin-3-yl)methyl)-2,6,6-trimethyl-6,7-dihydro-1H-indol-4(5H)-one (30.1 mg, 0.0870 mmol) was added, followed by copper(I) iodide (165 mg, 0.867 mmol). The reaction was heated to 125° C. for 25 minutes in the microwave, then diluted in saturated ammonium chloride, extracted with dichloromethane (3×30 mL), dried (sodium sulfate), filtered and concentrated. Purification by column chromatography on silica gel (Luknova 40 g, 20 mL/min) using 1 to 10% of 7:1 MeCN:MeOH in dichloromethane over 100 minutes afforded 2,6,6-trimethyl-34(2-(phenylsulfonyl)pyridin-3-yl)methyl)-6,7-dihydro-1H-indol-4(5H)-one (11.6 mg, 0.0280 mmol, 33% yield. ¹H NMR (400 MHz, CDCl₃) δ (ppm): 8.32 (d, 1H, J=4.4 Hz), 8.25 (br s, 1H), 8.05 (m, 2H), 7.50-7.62 (m, 3H), 7.43 (d, 2H, J=8.0 Hz), 7.19 (dd, 1H, J=8.0 Hz, J=4.4 Hz), 4.61 (s, 2H), 2.60 (s, 2H), 2.25 (s, 2H), 2.02 (s, 3H), 1.07 (s, 6H).

### Step 2

### Preparation of ethyl 2-(2,6,6-trimethyl-4-oxo-3-42-(phenylsulfonyl)pyridin-3-yl)methyl)-4,5,6,7-tetrahydro-1H-indol-1-yl)acetate (I-9)

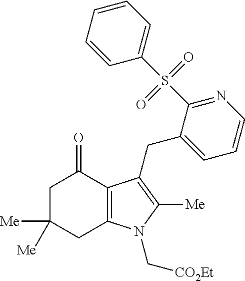 [SMILES: CCOC(=O)Cn1c(C)c(Cc2cccnc2S(=O)(=O)c2ccccc2)c2c1CC(C)(C)CC2=O]

To a suspension of 2,6,6-trimethyl-3-((2-(phenylsulfonyl)pyridin-3-yl)methyl)-6,7-dihydro-1H-indol-4(5H)-one (11.6 mg, 0.0280 mmol) in acetonitrile (3.5 ml) was added ethyl bromoacetate (0.0160 ml, 0.142 mmol), potassium carbonate (7.9 mg, 0.057 mmol), and potassium iodide (1.4 mg, 8.5 μmol). The reaction was heated to 95° C. for 12 hours (overnight), after which analysis by LCMS indicated about 75% conversion to the product. Additional ethyl bromoacetate (3 equiv.), potassium carbonate (2 equiv.) and potassium iodide (0.3 equiv.) were added and the reaction was heated to 110° C. for 3 additional hours. The reaction was then diluted in water, extracted with dichloromethane (3×30 mL), dried (sodium sulfate), filtered and concentrated to a residue which was purified on silica gel (Luknova 25 g, 20 mL/min) using 1 to 10% of 7:1 MeCN:MeOH over 100 minutes. The product ethyl 2-(2,6,6-trimethyl-4-oxo-3-((2-(phenylsulfonyl)pyridin-3-yl)methyl)-4,5,6,7-tetrahydro-1H-indol-1-yl)acetate (10.8 mg, 0.0220 mmol, 77% yield) was isolated as a solid. ¹H NMR (400 MHz, CDCl₃) δ (ppm): 8.35 (dd, 1H, J=4.4 Hz, J=0.8 Hz), 8.08 (m, 2H), 7.54-7.64 (m, 3H), 7.41 (dd, 1H, J=7.6 Hz, J=0.8 Hz), 7.21 (dd, 1H, J=7.6 Hz, J=4.4 Hz), 4.67 (s, 2H), 4.52 (s, 2H), 4.24 (q, 2H, J=7.2 Hz), 2.55 (s, 2H), 2.29 (s, 2H), 2.01 (s, 3H), 1.29 (t, 3H, J=7.2 Hz), 1.11 (s, 6H).

### Step 3

### Preparation of 2-(2,6,6-trimethyl-4-oxo-3-((2-(phenylsulfonyl)pyridin-3-yl)methyl)-4,5,6,7-tetrahydro-1H-indol-1-yl)acetic acid (I-3)

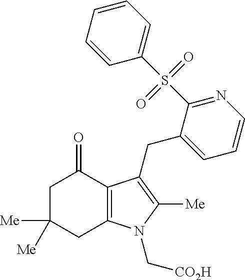 [SMILES: Cc1c(Cc2cccnc2S(=O)(=O)c2ccccc2)c2c(n1CC(=O)O)CC(C)(C)CC2=O]

To a solution of ethyl 2-(2,6,6-trimethyl-4-oxo-3-((2-(phenylsulfonyl)pyridin-3-yl)methyl)-4,5,6,7-tetrahydro-1H-indol-1-yl)acetate (16.8 mg, 0.0340 mmol) in THF (500 μL) and water (400 μL) was added a 1M solution of aqueous sodium hydroxide (67.9 μL, 0.0679 mmol). The reaction was stirred at room temperature for 10 minutes, after which analysis by LCMS indicated full conversion to the product. Neutralization with 6N hydrochloric acid (11.3 μL) followed by extraction with dichloromethane (3×15 mL), drying (sodium sulfate), filtering and concentration (in vacuo) afforded an off-white solid 2-(2,6,6-trimethyl-4-oxo-34(2-(phenylsulfonyl)pyridin-3-yl)methyl)-4,5,6,7-tetrahydro-1H-indol-1-yl)acetic acid (10.8 mg, 0.0230 mmol, 68% yield). ¹H NMR (400 MHz, CDCl₃) δ (ppm): 8.26 (m, 1H), 8.02 (d, 2H, J=7.6 Hz), 7.46-7.56 (m, 3H), 7.36 (m, 1H), 7.16 (m, 1H), 4.56 (s, 2H), 4.47 (s, 2H), 2.51 (s, 2H), 2.15 (s, 2H), 1.96 (s, 3H), 1.03 (s, 6H).

### Preparation of 2-(3-((2-(cyclopropylsulfonyl)pyridin-3-yl)methyl)-2,6,6-trimethyl-4-oxo-4,5,6,7-tetrahydro-1H-indol-1-yl)acetic acid (I-4)

### Step 1

### Preparation of 3-((2-(cyclopropylsulfonyl)pyridin-3-yl)methyl)-2,6,6-trimethyl-6,7-dihydro-1H-indol-4(5H)-one

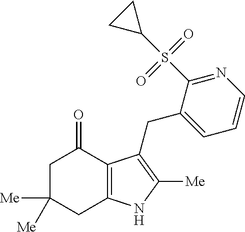 [SMILES: Cc1[nH]c2c(c1Cc1cccnc1S(=O)(=O)C1CC1)C(=O)CC(C)(C)C2]

To a microwave vial was added sodium cyclopropanesulfinate (55.3 mg, 0.432 mmol) and DMSO (0.9 ml). Solid 34(2-bromopyridin-3-yl)methyl)-2,6,6-trimethyl-6,7-dihydro-1H-indol-4(5H)-one (60.0 mg, 0.173 mmol) was added, followed by copper(I) iodide (329 mg, 1.73 mmol). The reaction was heated to 125° C. for 25 minutes in the microwave, then diluted in water and filtered through celite using first water then dichloromethane as the eluant. The biphasic solution was separated, and extracted with dichloromethane (3×30 mL), dried (sodium sulfate), filtered, and concentrated to a yellow residue. Purification by column chromatography using silica gel (ISCO 25 g, 20 mL/min) using 1 to 10% of 7:1 MeCN:MeOH in dichloromethane over 100 minutes affording 3-((2-(cyclopropylsulfonyl)pyridin-3-yl)methyl)-2,6,6-trimethyl-6,7-dihydro-1H-indol-4(5H)-one (30.2 mg, 0.0810 mmol, 47% yield) as a white solid. ¹H NMR (400 MHz, CDCl₃) δ (ppm): 8.45 (d, 1H, J=4.4 Hz), 7.87 (br s, 1H), 7.48 (d, 1H, J=6.8 Hz), 7.26-7.29 (m, 1H), 4.59 (s, 2H), 3.15-3.22 (m, 1H), 2.64 (s, 2H), 2.28 (s, 2H), 2.13 (s, 3H), 1.41-1.45 (m, 2H), 1.11-1.17 (m, 8H).

### Step 2

### Preparation of ethyl 2-(34(2-(cyclopropylsulfonyl)pyridin-3-yl)methyl)-2,6,6-trimethyl-4-oxo-4,5,6,7-tetrahydro-1H-indol-1-yl)acetate (I-54)

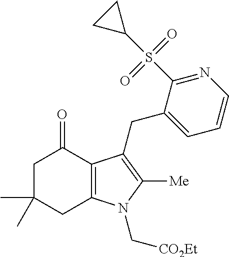 [SMILES: CCOC(=O)Cn1c(C)c(Cc2cccnc2S(=O)(=O)C2CC2)c2c1CC(C)(C)CC2=O]

To a solution of 34(2-(cyclopropylsulfonyl)pyridin-3-yl)methyl)-2,6,6-trimethyl-6,7-dihydro-1H-indol-4(5H)-one (30.2 mg, 0.0810 mmol) in acetonitrile (2 ml) was added ethyl bromoacetate (45.0 0.400 mmol) followed by potassium carbonate (22.4 mg, 0.162 mmol) and potassium iodide (4.0 mg, 0.024 mmol). The reaction was heated to 110° C. for 3 hours, after which LCMS showed it was complete. After cooling to room temperature, the reaction was extracted with dichloromethane (3×30 mL), dried (sodium sulfate), filtered and concentrated to a yellow residue which was purified by silica gel chromatography (Luknova 25 g, 20 mL/min) 1 to 10% of 7:1 MeCN:MeOH in dichloromethane over 100 minutes to afford ethyl 2434(2-(cyclopropylsulfonyl)pyridin-3-yl)methyl)-2,6,6-trimethyl-4-oxo-4,5,6,7-tetrahydro-1H-indol-1-yl)acetate (32.8 mg, 0.0720 mmol, 88% yield) as an off-white solid. ¹H NMR (400 MHz, CDCl₃) δ (ppm): 8.44 (dd, 1H, J=4.4 Hz, J=1.6 Hz), 7.43 (dd, 1H, J=8.0 Hz, J=1.6 Hz), 7.27 (dd, 1H, J=8.0 Hz, J=4.4 Hz), 4.62 (s, 2H), 4.53 (s, 2H), 4.24 (q, 2H, J=7.2 Hz), 3.14-3.21 (m, 1H), 2.55 (s, 2H), 2.28 (s, 2H), 2.05 (s, 3H), 1.40-1.45 (m, 2H), 1.29 (t, 3H, J=7.2 Hz), 1.11-1.16 (m, 8H).

### Step 3

### Preparation of 2-(3-((2-(cyclopropylsulfonyl)pyridin-3-yl)methyl)-2,6,6-trimethyl-4-oxo-4,5,6,7-tetrahydro-1H-indol-1-yl)acetic acid (I-4)

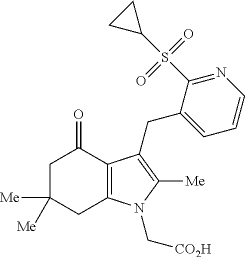 [SMILES: Cc1c(Cc2cccnc2S(=O)(=O)C2CC2)c2c(n1CC(=O)O)CC(C)(C)CC2=O]

To a solution of ethyl 2-(3-((2-(cyclopropylsulfonyl)pyridin-3-yl)methyl)-2,6,6-trimethyl-4-oxo-4,5,6,7-tetrahydro-1H-indol-1-yl)acetate (32.8 mg, 0.0720 mmol) in THF (0.5 ml) and water (0.5 ml) was added a 1M aqueous sodium hydroxide solution (0.143 ml, 0.143 mmol). After 5 minutes, the reaction was determined to be complete by LCMS, and neutralized with 1M hydrochloric acid (0.143 mL), concentrated to about 50% volume, and filtered. The solids were washed with water and dried to afford 2-(34(2-(cyclopropylsulfonyl)pyridin-3-yl)methyl)-2,6,6-trimethyl-4-oxo-4,5,6,7-tetrahydro-1H-indol-1-yl)acetic acid (22.6 mg, 0.0520 mmol, 73% yield). ¹H NMR (400 MHz, CDCl₃) δ (ppm): 8.35 (dd, 1H, J=4.4 Hz, J=0.8 Hz), 7.35 (dd, 1H, J=8.0 Hz, J=0.8 Hz), 7.23 (dd, 1H, J=8.0 Hz, J=4.4 Hz), 4.50 (s, 2H), 4.46 (s, 2H), 3.05-3.12 (m, 1H), 2.50 (s, 2H), 2.18 (s, 2H), 1.98 (s, 3H), 1.30-1.34 (m, 2H), 1.05-1.10 (m, 2H), 1.03 (s, 6H).

### Preparation of 2-(34(2-(4-fluorophenylsulfonyl)pyridin-3-yl)methyl)-2,6,6-trimethyl-4-oxo-4,5,6,7-tetrahydro-1H-indol-1-yl)acetic acid (I-5)

### Step 1

### Preparation of 3-((2-(4-fluorophenylsulfonyl)pyridin-3-yl)methyl)-2,6,6-trimethyl-6,7-dihydro-1H-indol-4(5H)-one

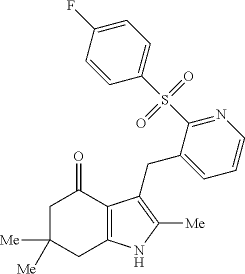 [SMILES: Cc1[nH]c2c(c1Cc1cccnc1S(=O)(=O)c1ccc(F)cc1)C(=O)CC(C)(C)C2]

To a microwave vial was added sodium 4-fluorobenzenesulfinate (55.1 mg, 0.302 mmol) and DMSO (0.9 ml). Solid 3-((2-bromopyridin-3-yl)methyl)-2,6,6-trimethyl-6,7-dihydro-1H-indol-4(5H)-one (42.0 mg, 0.121 mmol) was added, followed by copper(I) iodide (230 mg, 1.21 mmol). The reaction was heated to 125° C. for 25 minutes in the microwave. Analysis by LCMS indicated the presence of the desired product and some starting material. The reaction was resubjected to the same reaction microwave heating conditions, then diluted in water and filtered through celite using dichloromethane and ethyl acetate (successively) as the eluant. Extraction with dichloromethane (3×30 mL), followed by purification on silica gel (Luknova 25 g, 20 mL/min) using 1 to 10% of 7:1 MeCN:MeOH in dichloromethane over 100 minutes afforded 3-((2-(4-fluorophenylsulfonyl)pyridin-3-yl)methyl)-2,6,6-trimethyl-6,7-dihydro-1H-indol-4(5H)-one (30.2 mg, 0.0710 mmol, 59% yield) as an off-white solid. ¹H NMR (400 MHz, CDCl₃) δ (ppm): 8.32 (br s, 1H), 8.11 (dd, 2H, J=8.4 Hz, J=4.8 Hz), 8.00 (m, 1H), 7.46 (d, 1H, J=8.0 Hz), 7.20-7.26 (m, 3H), 4.65 (s, 2H), 2.64 (s, 2H), 2.27 (s, 2H), 2.10 (s, 3H), 1.11 (s, 6H).

### Step 2

### Preparation of ethyl 2-(3-((2-(4-fluorophenylsulfonyl)pyridin-3-yl)methyl)-2,6,6-trimethyl-4-oxo-4,5,6,7-tetrahydro-1H-indol-1-yl)acetate (I-8)

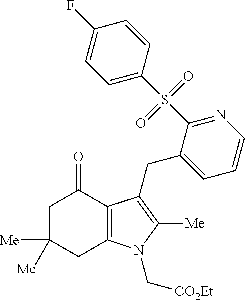 [SMILES: CCOC(=O)Cn1c(C)c(Cc2cccnc2S(=O)(=O)c2ccc(F)cc2)c2c1CC(C)(C)CC2=O]

To a solution of 3-((2-(4-fluorophenylsulfonyl)pyridin-3-yl)methyl)-2,6,6-trimethyl-6,7-dihydro-1H-indol-4(5H)-one (11.5 mg, 0.0270 mmol) in acetonitrile (1 ml) was added ethyl bromoacetate (0.015 ml, 0.14 mmol), potassium iodide (1.3 mg, 8.1 μmol), and potassium carbonate (7.5 mg, 0.054 mmol). The reaction was heated to 110° C. After two hours, the reaction was cooled to room temperature, diluted in water, extracted with dichloromethane (3×30 mL), dried (sodium sulfate), and concentrated to an oily residue ethyl 2-(3-((2-(4-fluorophenylsulfonyl)pyridin-3-yl)methyl)-2,6,6-trimethyl-4-oxo-4,5,6,7-tetrahydro-1H-indol-1-yl)acetate (12 mg, 0.024 mmol, 88% yield) which was used in the next step without purification. ¹H NMR (400 MHz, CDCl₃) δ (ppm): 8.32 (dd, 1H, J=4.8 Hz, J=1.6 Hz), 8.10-8.14 (m, 2H), 7.40 (m, 1H), 7.20-7.26 (m, 3H), 4.69 (s, 2H), 4.55 (s, 2H), 4.25 (q, 2H, J=7.2 Hz), 2.56 (s, 2H), 2.29 (s, 2H), 2.05 (s, 3H), 1.26 (m, 3H), 1.12 (s, 6H).

### Step 3

### Preparation of 2-(3-((2-(4-fluorophenylsulfonyl)pyridin-3-yl)methyl)-2,6,6-trimethyl-4-oxo-4,5,6,7-tetrahydro-1H-indol-1-yl)acetic acid (I-5)

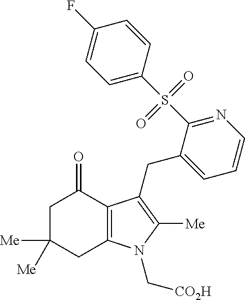 [SMILES: Cc1c(Cc2cccnc2S(=O)(=O)c2ccc(F)cc2)c2c(n1CC(=O)O)CC(C)(C)CC2=O]

To a solution of ethyl 2-(3-((2-(4-fluorophenylsulfonyl)pyridin-3-yl)methyl)-2,6,6-trimethyl-4-oxo-4,5,6,7-tetrahydro-1H-indol-1-yl)acetate (12.1 mg, 0.0240 mmol) in THF (0.2 ml) and water (0.15 ml) was added an aqueous 1M solution of sodium hydroxide (0.047 ml, 0.047 mmol). After 15 minutes, analysis by LCMS revealed completion of the reaction to the desired product. Neutralization with aqueous 1M hydrochloric acid (0.047 mL), followed by purification using reverse phase semi-preparative HPLC (10 to 90% MeCN in water spiked with 0.1% trifluoroacetic acid in both solvents-50 minute gradient) afforded the desired product 2-(3-((2-(4-fluorophenylsulfonyl)pyridin-3-yl)methyl)-2,6,6-trimethyl-4-oxo-4,5,6,7-tetrahydro-1H-indol-1-yl)acetic acid (4.4 mg, 9.1 μmol, 39% yield) as an off-white solid. ¹H NMR (400 MHz, CDCl₃) δ (ppm): 8.34 (d, 1H, J=3.6 Hz), 8.08 (m, 2H), 7.38 (d, 1H, J=7.6 Hz), 7.21-7.26 (m, 3H), 4.62 (s, 2H), 4.61 (s, 2H), 2.58 (s, 2H), 2.32 (s, 2H), 2.06 (s, 3H), 1.10 (s, 6H).

### Preparation of 2-(3-((2-(4-methoxyphenylsulfonyl)pyridin-3-yl)methyl)-2,6,6-trimethyl-4-oxo-4,5,6,7-tetrahydro-1H-indol-1-yl)acetic acid (I-7)

### Step 1

### Preparation of 3-((2-(4-methoxyphenylsulfonyl)pyridin-3-yl)methyl)-2,6,6-trimethyl-6,7-dihydro-1H-indol-4(5H)-one

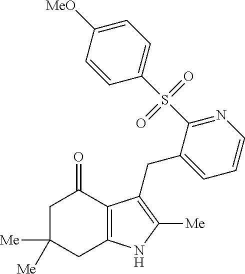 [SMILES: COc1ccc(S(=O)(=O)c2ncccc2Cc2c(C)[nH]c3c2C(=O)CC(C)(C)C3)cc1]

A solution of 3-((2-bromopyridin-3-yl)methyl)-2,6,6-trimethyl-6,7-dihydro-1H-indol-4(5H)-one (150 mg, 0.432 mmol), copper(I) iodide (411 mg, 2.16 mmol), and sodium 4-methoxybenzenesulfinate (210 mg, 1.08 mmol) in DMSO (3 ml) was heated in the microwave for 25 minutes at 125° C. The reaction was quenched by the addition of water and filtered through a pad of celite with ethyl acetate as the eluant. The filtrate was extracted with ethyl acetate (3×30 mL), dried (sodium sulfate), filtered and concentrated to a tan residue which was purified on silica gel (ISCO 40 g, 20 mL/min) using 1 to 7% 7:1 MeCN:MeOH in dichloromethane over 75 minutes. Repurification using 40-100% ethyl acetate in hexanes over 50 minutes afforded 3-((2-(4-methoxyphenylsulfonyl)pyridin-3-yl)methyl)-2,6,6-trimethyl-6,7-dihydro-1H-indol-4(5H)-one (52.7 mg, 0.120 mmol, 28% yield). ¹H NMR (400 MHz, CDCl₃) δ (ppm): 8.34 (dd, 1H, J=4.4 Hz, J=1.2 Hz), 8.01 (dd, 2H, J=6.8 Hz, J=2.0 Hz) 7.88 (br s, 1H), 7.45 (d, 1H, J=8.0 Hz), 7.20 (dd, 1H, J=8.0 Hz, J=4.4 Hz), 7.02 (dd, 2H, J=6.8 Hz, J=2.0 Hz), 4.66 (s, 2H), 3.87 (s, 3H), 2.64 (s, 2H), 2.29 (s, 2H), 2.10 (s, 3H), 1.12 (s, 6H).

### Step 2

### Preparation of ethyl 2-(3-((2-(4-methoxyphenylsulfonyl)pyridin-3-yl)methyl)-2,6,6-trimethyl-4-oxo-4,5,6,7-tetrahydro-1H-indol-1-yl)acetate (I-6)

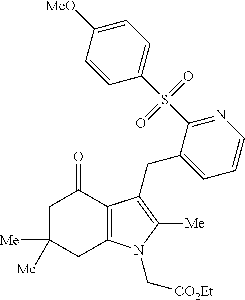 [SMILES: CCOC(=O)Cn1c(C)c(Cc2cccnc2S(=O)(=O)c2ccc(OC)cc2)c2c1CC(C)(C)CC2=O]

To a suspension of 3-((2-(4-methoxyphenylsulfonyl)pyridin-3-yl)methyl)-2,6,6-trimethyl-6,7-dihydro-1H-indol-4(5H)-one (52.7 mg, 0.120 mmol) in acetonitrile (4 ml) was added ethyl 2-bromoacetate (0.0540 ml, 0.481 mmol), potassium carbonate (33.2 mg, 0.240 mmol), and potassium iodide (5.98 mg, 0.0360 mmol). The reaction was sealed and heated to 100° C. for 12 hours, after which it was cooled to room temperature, diluted with water, extracted with dichloromethane (3×30 mL), dried (sodium sulfate), filtered, and concentrated to a tan residue. Purification by column chromatography on silica gel (Luknova 12 g, 20 mL/min) using 1 to 7% of 7:1 MeCN:MeOH in dichloromethane over 100 minutes furnished ethyl 2-(3-((2-(4-methoxyphenylsulfonyl)pyridin-3-yl)methyl)-2,6,6-trimethyl-4-oxo-4,5,6,7-tetrahydro-1H-indol-1-yl)acetate (43.1 mg, 0.0820 mmol, 68% yield) as an off-white solid. ¹H NMR (400 MHz, CDCl₃) δ (ppm): 8.34 (dd, 1H, J=4.4 Hz, 1.2 Hz), 8.02 (dd, 2H, J=6.8 Hz, J=2.0 Hz), 7.40 (dd, 1H, J=8.0 Hz, J=1.2 Hz), 7.20 (dd, 1H, J=7.6 Hz, J=4.4 Hz), 7.02 (dd, 2H, J=7.2 Hz, J=2.0 Hz), 4.69 (s, 2H), 4.53 (s, 2H), 4.25 (q, 2H, J=7.2 Hz), 3.87 (s, 3H), 2.56 (s, 2H), 2.30 (s, 2H), 2.02 (s, 3H), 1.29 (t, 3H, J=7.2 Hz), 1.12 (s, 6H).

### Step 3

### Preparation of 2-(3-((2-(4-methoxyphenylsulfonyl)pyridin-3-yl)methyl)-2,6,6-trimethyl-4-oxo-4,5,6,7-tetrahydro-1H-indol-1-yl)acetic acid (I-7)

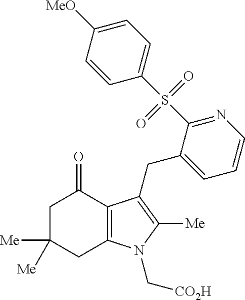 [SMILES: COc1ccc(S(=O)(=O)c2ncccc2Cc2c3c(n(CC(=O)O)c2C)CC(C)(C)CC3=O)cc1]

To a solution of ethyl 2-(3-((2-(4-methoxyphenylsulfonyl)pyridin-3-yl)methyl)-2,6,6-trimethyl-4-oxo-4,5,6,7-tetrahydro-1H-indol-1-yl)acetate (5.0 mg, 9.5 μmol) in water (100 μL) and THF (100 μL) was added an aqueous 1M solution of sodium hydroxide (19 μL, 0.019 mmol). The reaction was stirred at room temperature for 20 minutes, after which the reaction was quenched by the addition of aqueous 1M hydrochloric acid (19 μL), concentrated to 1/3 volume, then diluted in water (0.5 mL). An off-white precipitate formed, which was filtered and dried in vacuum to afford 2-(3-((2-(4-methoxyphenylsulfonyl)pyridin-3-yl)methyl)-2,6,6-trimethyl-4-oxo-4,5,6,7-tetrahydro-1H-indol-1-yl)acetic acid (2.8 mg, 5.6 μmol, 59% yield). ¹H NMR (400 MHz, CD₃CN) δ (ppm): 8.36 (d, 1H, J=4.8 Hz), 8.07 (dd, 2H, J=8.8 Hz, J=1.6 Hz), 7.48 (d, 1H, J=8.0 Hz), 7.43 (dd, 1H, J=8.0 Hz, J=4.4 Hz), 7.25 (dd, 2H, J=8.8 Hz, J=1.6 Hz), 4.74 (s, 2H), 4.71 (s, 2H), 4.00 (s, 3H), 2.72 (s, 2H), 2.33 (s, 2H), 2.06 (s, 3H), 1.20 (s, 6H).

The following compounds were prepared following analogous procedures to those exemplified in Example 2 and General Procedure B

### Compound I-32

### 2-(2,6,6-Trimethyl-3-((2-(methylsulfonyl)pyridin-3-yl)methyl)-4-oxo-4,5,6,7-tetrahydro-1H-indol-1-yl)acetic acid (I-32)

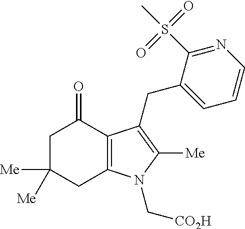 [SMILES: Cc1c(Cc2cccnc2S(C)(=O)=O)c2c(n1CC(=O)O)CC(C)(C)CC2=O]

¹H NMR (400 MHz, CD₃OD) δ (ppm): 8.31 (d, 2, H), 7.36 (m, 2H), 4.62 (s, 2H), 4.43 (s, 2H), 3.31 (s, 3H), 2.56 (s, 2H), 2.15 (s, 2H), 1.95 (s, 3H), 1.01 (s, 6H).

### Compound I-13

### Step 2

### Ethyl 2-(3-((2-(3-methoxyphenylsulfonyl)pyridin-3-yl)methyl)-2,6,6-trimethyl-4-oxo-4,5,6,7-tetrahydro-1H-indol-1-yl)acetate (I-11)

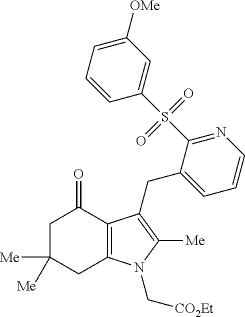 [SMILES: CCOC(=O)Cn1c(C)c(Cc2cccnc2S(=O)(=O)c2cccc(OC)c2)c2c1CC(C)(C)CC2=O]

¹H NMR (CDCl₃/400 MHz): δ (ppm): 8.49-8.50 (m, 1H), 7.62 (d, 1H), 7.56 (br. S, 1H), 7.40-7.47 (m, 2H), 7.32-7.35 (m, 1H), 7.15 (dd, 1H), 4.58 (s, 2H), 4.55 (s, 2H), 4.26 (q, 2H), 3.85 (s, 3H), 2.58 (s, 2H), 2.38 (s, 2H), 2.01 (s, 3H), 1.30 (t, 3H), 1.12 (s, 6H).

### Step 3

### 2-(3-((2-(3-Methoxyphenylsulfonyl)pyridin-3-yl)methyl)-2,6,6-trimethyl-4-oxo-4,5,6,7-tetrahydro-1H-indol-1-yl)acetic acid (I-13)

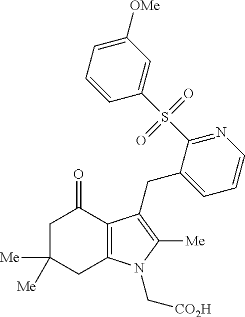 [SMILES: COc1cccc(S(=O)(=O)c2ncccc2Cc2c3c(n(CC(=O)O)c2C)CC(C)(C)CC3=O)c1]

¹H NMR (CDCl₃/400 MHz): δ (ppm): 8.37-8.38 (m, 1H), 7.61 (d, 1H), 7.55 (br. s, 1H), 7.45 (t, 1H), 7.38 (d, 1H), 7.23-7.24 (m, 1H), 7.15 (dd, 1H), 5.5 (br. s, 1H), 4.61 (s, 2H), 4.59 (s, 2H), 3.85 (s, 3H), 2.57 (s, 2H), 2.32 (s, 2H), 2.01 (s, 3H), 1.25 (s, 3H), 1.1 (s, 3H).

### Compound I-15

### Step 2

### Ethyl 2-(3-((2-(4-chlorophenylsulfonyl)pyridin-3-yl)methyl)-2,6,6-trimethyl-4-oxo-4,5,6,7-tetrahydro-1H-indol-1-yl)acetate (I-14)

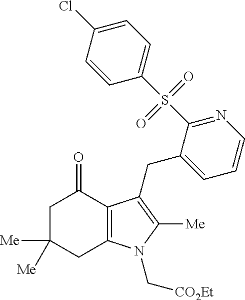 [SMILES: CCOC(=O)Cn1c(C)c(Cc2cccnc2S(=O)(=O)c2ccc(Cl)cc2)c2c1CC(C)(C)CC2=O]

¹H NMR (CDCl₃/400 MHz): δ (ppm): 8.34-8.35 (m, 1H), 8.01 (d, 2H), 7.53 (d, 2H), 7.38 (d, 1H), 7.23-7.27 (m, 1H), 4.64 (s, 2H), 4.55 (s, 2H), 4.26 (q, 2H), 2.57 (s, 2H), 2.36 (s, 2H), 2.05 (s, 3H), 1.30 (t, 3H), 1.12 (s, 6H).

### Step 3

### 2-(3-((2-(4-Chlorophenylsulfonyl)pyridin-3-yl)methyl)-2,6,6-trimethyl-4-oxo-4,5,6,7-tetrahydro-1H-indol-1-yl)acetic acid (I-15)

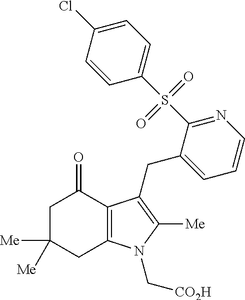 [SMILES: Cc1c(Cc2cccnc2S(=O)(=O)c2ccc(Cl)cc2)c2c(n1CC(=O)O)CC(C)(C)CC2=O]

¹H NMR (CDCl₃/400 MHz): δ (ppm): 8.24-8.25 (m, 1H), 7.99 (d, 2H), 7.52 (d, 2H), 7.39 (d, 1H), 7.18-7.21 (m, 1H), 4.62 (s, 2H), 4.53 (s, 2H), 2.54 (s, 2H), 2.23 (s, 2H), 2.01 (s, 3H), 1.25 (s, 3H), 1.07 (s, 3H).

### Compound I-20

### Step 2

### Ethyl 2-(2,6,6-trimethyl-4-oxo-3-((2-tosylpyridin-3-yl)methyl)-4,5,6,7-tetrahydro-1H-indol-1-yl)acetate (I-16)

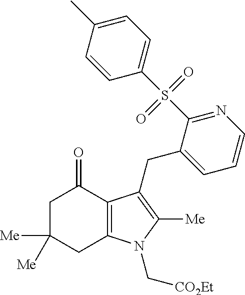 [SMILES: CCOC(=O)Cn1c(C)c(Cc2cccnc2S(=O)(=O)c2ccc(C)cc2)c2c1CC(C)(C)CC2=O]

¹H NMR (CDCl₃/400 MHz): δ (ppm): 8.38-8.39 (m, 1H), 7.94 (d, 2H), 7.33-7.38 (m, 3H), 7.22-7.26 (m, 1H), 4.64 (s, 2H), 4.54 (s, 2H), 4.24 (q, 2H), 2.57 (s, 2H), 2.42 (s, 3H), 2.34-3.36 (m, 2H), 2.01 (s, 3H), 1.29 (t, 3H), 1.11 (s, 6H).

### Step 3

### 2-(2,6,6-Trimethyl-4-oxo-3-((2-tosylpyridin-3-yl)methyl)-4,5,6,7-tetrahydro-1H-indol-1-yl)acetic acid (I-20)

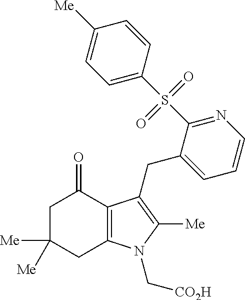 [SMILES: Cc1ccc(S(=O)(=O)c2ncccc2Cc2c3c(n(CC(=O)O)c2C)CC(C)(C)CC3=O)cc1]

¹H NMR (400 MHz, CDCl₃) δ (ppm): 8.35 (d, 1H), 7.96 (d, 2H), 7.43 (d, 1H), 7.35 (d, 2H), 7.19-7.25 (m, 1H), 4.68 (s, 2H), 4.58 (s, 2H), 2.58 (s, 2H), 2.44 (s, 3H), 2.31 (s, 2H), 2.05 (s, 3H), 1.13 (s, 6H).

### Compound I-17

### 2-(2,6,6-Trimethyl-4-oxo-3-((2-(2-(trifluoromethyl)phenylsulfonyl)pyridin-3-yl)methyl)-4,5,6,7-tetrahydro-1H-indol-1-yl)acetic acid (I-17)

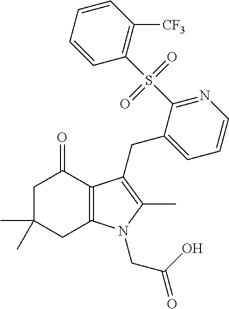 [SMILES: Cc1c(Cc2cccnc2S(=O)(=O)c2ccccc2C(F)(F)F)c2c(n1CC(=O)O)CC(C)(C)CC2=O]

¹H NMR (400 MHz, CD₃OD) δ (ppm): 7.32-8.34 (m, 1H), 8.02 (d, 1H), 7.82-7.89 (m, 3H), 7.39 (d, 1H), 7.21-724 (m, 1H), 4.62 (s, 2H), 4.56 (s, 2H), 2.58 (s, 2H), 2.21 (s, 2H), 1.96 (s, 3H), 1.04 (s, 6H)

### Compound I-12

### Step 2

### Ethyl 2-(2,6,6-trimethyl-4-oxo-3-((2-(4-(trifluoromethyl) phenylsulfonyl)pyridin-3-yl)methyl)-4,5,6,7-tetrahydro-1H-indol-1-yl)acetate (I-10)

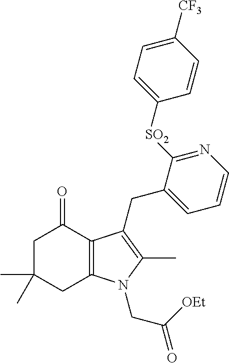 [SMILES: CCOC(=O)Cn1c(C)c(Cc2cccnc2S(=O)(=O)c2ccc(C(F)(F)F)cc2)c2c1CC(C)(C)CC2=O]

¹H NMR (400 MHz, Acetone D₆) δ (ppm): 7.95 (d, 2H), 7.89 (d, 1H), 7.68 (d, 2H), 7.12-7.25 (m, 1H), 7.03-7.06 (m, 1H), 4.46 (s, 2H), 4.38 (s, 2H), 3.83-3.88 (q, 2H), 2.93 (s, 3H), 2.46 (d, 2H), 1.85 (s, 2H), 1.71 (s, 3H), 0.74 (s, 6H)

### Step 3

### 2-(2,6,6-Trimethyl-4-oxo-3-((2-(4-(trifluoromethyl)phenylsulfonyl)pyridin-3-yl)methyl)-4,5,6,7-tetrahydro-1H-indol-1-yl)acetic acid (I-12)

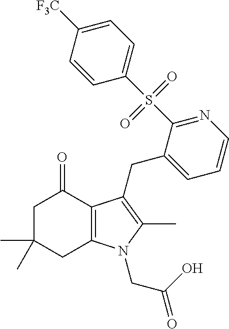 [SMILES: Cc1c(Cc2cccnc2S(=O)(=O)c2ccc(C(F)(F)F)cc2)c2c(n1CC(=O)O)CC(C)(C)CC2=O]

¹H NMR (400 MHz, Acetone D₆) δ (ppm): 8.32 (d, 2H), 8.25 (d, 1H), 8.05 (d, 2H), 7.50 (d, 1H), 7.39-7.42 (m, 1H), 4.81 (s, 2H), 4.74 (s, 2H), 2.72 (s, 2H), 2.21 (s, 2H), 2.10 (s, 3H), 1.10 (s, 6H)

### Compound I-1

### Step 2

### Ethyl 2-(3-((2-(2,4-difluorophenylsulfonyl)pyridin-3-yl)methyl)-2,6,6-trimethyl-4-oxo-4,5,6,7-tetrahydro-1H-indol-1-yl)acetate (I-18)

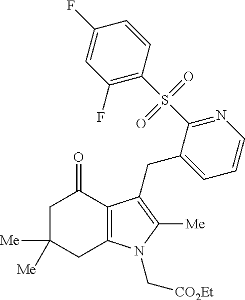 [SMILES: CCOC(=O)Cn1c(C)c(Cc2cccnc2S(=O)(=O)c2ccc(F)cc2F)c2c1CC(C)(C)CC2=O]

¹H NMR (400 MHz, CDCl₃) δ (ppm): 8.27 (d, 1H), 8.15 (m, 1H), 7.50 (d, 1H), 7.24 (m, 1H), 7.07 (m, 1H), 6.95 (m, 1H), 4.70 (s, 2H), 4.54 (s, 2H), 4.25 (q, 2H), 2.57 (s, 2H), 2.33 (s, 2H), 2.07 (s, 3H), 1.22 (t, 3H), 0.95 (s, 6H).

### Step 3

### 2-(3-((2-(2,4-Difluorophenylsulfonyl)pyridin-3-yl)methyl)-2,6,6-trimethyl-4-oxo-4,5,6,7-tetrahydro-1H-indol-1-yl)acetic acid (I-19)

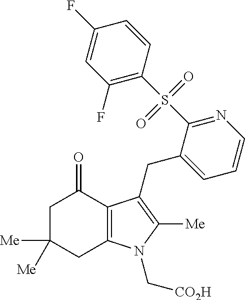 [SMILES: Cc1c(Cc2cccnc2S(=O)(=O)c2ccc(F)cc2F)c2c(n1CC(=O)O)CC(C)(C)CC2=O]

¹H NMR (400 MHz, CDCl₃) δ (ppm): 8.09 (d, 1H), 8.04 (m, 1H), 7.37 (m, 1H), 7.14 (m, 1H), 7.02 (m, 1H), 6.87 (m, 1H), 4.49 (s, 2H), 4.38 (s, 2H), 2.46 (s, 2H), 2.08 (s, 2H), 1.95 (s, 3H), 0.95 (s, 6H).

### Compound I-21

### 2-(3-((2-(3-Fluoro-4-methoxyphenylsulfonyl)pyridin-3-yl)methyl)-2,6,6-trimethyl-4-oxo-4,5,6,7-tetrahydro-1H-indol-1-yl)acetic acid (I-21)

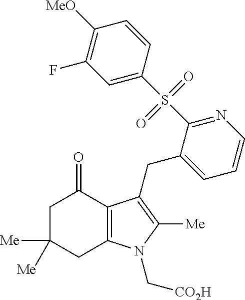 [SMILES: COc1ccc(S(=O)(=O)c2ncccc2Cc2c3c(n(CC(=O)O)c2C)CC(C)(C)CC3=O)cc1F]

¹H NMR (400 MHz, CDCl₃) δ (ppm): 8.33 (d, 1H), 7.85 (d, 1H), 7.79 (dd, 1H), 7.44 (d, 1H), 7.20-7.23 (m, 1H), 7.10 (m, 1H), 4.68 (s, 2H), 4.59 (s, 2H), 3.97 (s, 3H), 2.58 (s, 2H), 2.31 (s, 2H), 2.07 (s, 3H), 1.12 (s, 6H).

### Compound I-26

### 2-(2,6,6-Trimethyl-4-oxo-3-((5-(phenylsulfonyl)thiophen-2-yl)methyl)-4,5,6,7-tetrahydro-1H-indol-1-yl)acetic acid (I-26)

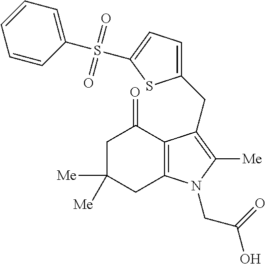 [SMILES: Cc1c(Cc2ccc(S(=O)(=O)c3ccccc3)s2)c2c(n1CC(=O)O)CC(C)(C)CC2=O]

¹H NMR (400 MHz; CDCl₃) δ (ppm): 7.91 (d, 1H), 7.52 (m, 1H), 7.47 (m, 2H), 6.85 (d, 1H), 4.56 (s, 2H), 4.27 (s, 2H), 2.53 (s, 2H), 2.31 (s, 2H), 2.11 (s, 3H), 1.09 (s, 6H).

### Compound I-42

### 2-(2,6,6-Trimethyl-4-oxo-34(3-(phenylsulfonyl)thiophen-2-yl)methyl)-4,5,6,7-tetrahydro-1H-indol-1-yl)acetic acid (I-42)

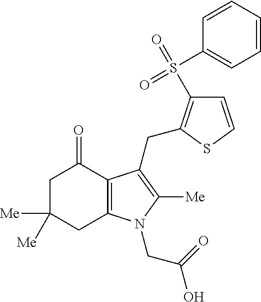 [SMILES: Cc1c(Cc2sccc2S(=O)(=O)c2ccccc2)c2c(n1CC(=O)O)CC(C)(C)CC2=O]

¹H NMR (400 MHz, CDCl₃) δ (ppm): 8.04 (d, 2H), 7.55 (m, 3H), 7.31 (m, 2H), 7.00 (d, 1H), 4.59 (s, 2H), 4.57 (s, 2H), 2.55 (s, 2H), 2.29 (s, 2H), 2.06 (s, 3H), 1.11 (s, 6H).

### Example 3

### Preparation of Fused Heterocyclic Pyrroles

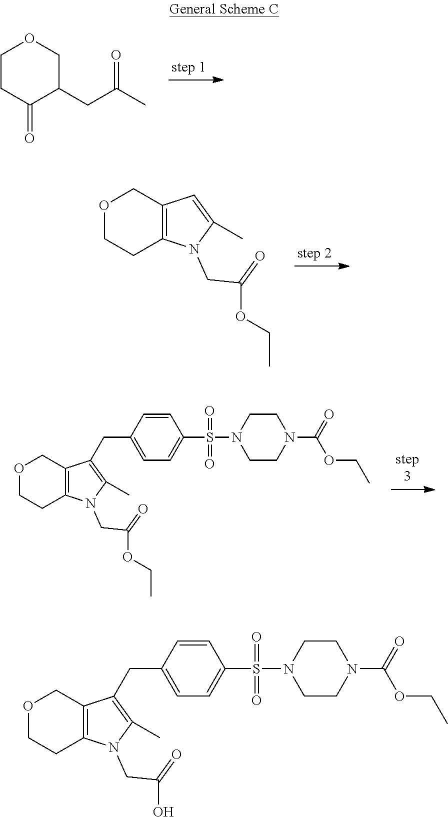 [SMILES: CC(=O)CC1COCCC1=O ; CCOC(=O)Cn1c(C)cc2c1CCOC2 ; CCOC(=O)Cn1c(C)c(Cc2ccc(S(=O)(=O)N3CCN(C(=O)OCC)CC3)cc2)c2c1CCOC2 ; CCOC(=O)N1CCN(S(=O)(=O)c2ccc(Cc3c4c(n(CC(=O)O)c3C)CCOC4)cc2)CC1]

### Preparation of 2-(3-(4-(4-(ethoxycarbonyl)piperazin-1-ylsulfonyl)benzyl)-2-methyl-6,7-dihydropyrano[4,3-b]pyrrol-1(4H)-yl)acetic acid (I-2)

### Starting Material Preparation

### 3-(2-Oxopropyl)dihydro-2H-pyran-4(3H)-one

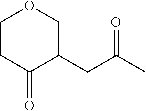

(3,6-Dihydro-2H-pyran-4-yloxy)trimethylsilane (1.00 g, 5.80 mmol) and trimethyl(prop-1-en-2-yloxy)silane (7.56 g, 58.0 mmol) were added dropwise to a vigorously stirred suspension of eerie ammonium nitrate (6.36 g, 11.61 mmol) and sodium bicarbonate (1.950 g, 23.22 mmol) in dry acetonitrile (40 ml) stirred until the orange color disappeared and a thick white precipitate formed. The reaction mix was poured into water and extracted with EtOAc, combined extracts were washed with brine, dried over Na₂SO₄, filtered and concentrated. The residue was purified by column, EtOAc/Hex 10-50% to give 0.42 g, yield 46.3%. ¹H NMR (400 MHz, CDCl₃) δ (ppm): 4.30-4.25 (m, 1H), 4.21-4.16 (m, 1H), 3.68-3.63 (m, 1H), 3.34 (t, 1H), 3.19-3.15 (m, 1H), 2.91-2.85 (m, 1H), 2.75-2.66 (m, 1H), 2.39-2.34 (m, 1H), 2.21-2.15 (m, 4H)

### Step 1

### Ethyl 2-(2-methyl-6,7-dihydropyrano[4,3-b]pyrrol-1(4H)-yl)acetate

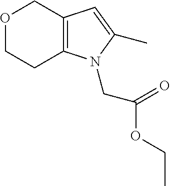 [SMILES: CCOC(=O)Cn1c(C)cc2c1CCOC2]

3-(2-Oxopropyl)dihydro-2H-pyran-4(3H)-one (0.41 g, 2.63 mmol) in DCM (3 ml) was stirred, ethyl 2-aminoacetate hydrochloride (0.366 g, 2.63 mmol) was added, followed by sodium bicarbonate (0.441 g, 5.25 mmol), the resulting mixture was stirred at 25° C. for 15 hours, TLC showed that reaction was complete. DCM was added, washed with water, brine, dried with NaHSO₄, filtered and concentrated, the residue was chromatographed by Biotage, EtOAc/Hexane 5-30%, to give 0.41 g, yield 70%. ¹H NMR (400 MHz, CDCl₃) δ (ppm): 5.69 (s, 1H), 4.62 (s, 2H), 4.44 (s, 2H), 4.21 (q, 2H), 3.96-3.94 (m, 2H), 2.58-2.55 (m, 2H), 2.18 (s, 3H), 1.28 (t, 3H).

### Step 2

### Ethyl 4-(4-((1-(2-ethoxy-2-oxoethyl)-2-methyl-1,4,6,7-tetrahydropyrano[4,3-b]pyrrol-3-yl)methyl)phenylsulfonyl)piperazine-1-carboxylate (I-55)

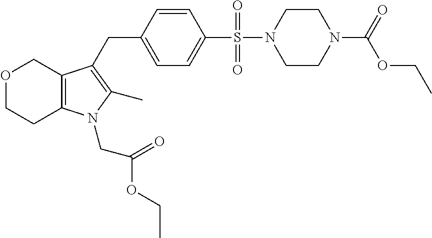 [SMILES: CCOC(=O)Cn1c(C)c(Cc2ccc(S(=O)(=O)N3CCN(C(=O)OCC)CC3)cc2)c2c1CCOC2]

Ethyl 2-(2-methyl-6,7-dihydropyrano[4,3-b]pyrrol-1(4H)-yl)acetate (50 mg, 0.224 mmol) and ethyl 4-(4-(bromomethyl)phenylsulfonyl)piperazine-1-carboxylate (88 mg, 0.224 mmol) in acetonitrile (2 ml) was stirred, potassium carbonate (61.9 mg, 0.448 mmol) was added, the mixture was stirred at 100° C. for 24 hours, cooled to r.t. and purified by Biotage, EtOAc/Hex, 5-30-50%, to give 25 mg, 0.047 mmol, yield 21%). ¹H NMR (400 MHz, CDCl₃): δ (ppm): 7.62 (d, 2H), 7.28 (d, 2H), 4.47 (s, 2H), 4.39 (s, 2H), 4.22 (q, 2H), 3.92 (q, 2H), 3.76-3.72 (m, 6H), 3.01-2.96 (m, 4H), 2.57 (t, 2H), 2.11 (s, 3H), 1.30 (t, 6H)

### Step 3

### 2-(3-(4-(4-(Ethoxycarbonyl)piperazin-1-ylsulfonyl)benzyl)-2-methyl-6,7-dihydropyrano[4,3-b]pyrrol-1(4H)-yl)acetic acid (I-2)

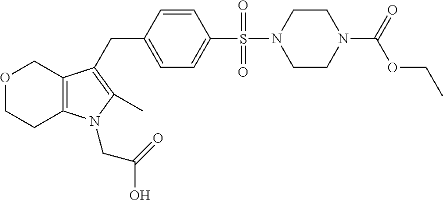 [SMILES: CCOC(=O)N1CCN(S(=O)(=O)c2ccc(Cc3c4c(n(CC(=O)O)c3C)CCOC4)cc2)CC1]

Ethyl 4-(4-((1-(2-ethoxy-2-oxoethyl)-2-methyl-1,4,6,7-tetrahydropyrano[4,3-b]pyrrol-3-yl)methyl)phenylsulfonyl)piperazine-1-carboxylate (25 mg, 0.047 mmol) in THF (2.0 ml), MeOH (0.500 ml) and water (0.50 ml) was stirred, then LiOH (2.244 mg, 0.094 mmol) was added, stirred at 25° C. for two hours, TLC showed that the starting material had disappeared. Purification by prep-HPLC gave a light blue solid. 2-(3-(4-(4-(ethoxycarbonyl)piperazin-1-ylsulfonyl)benzyl)-2-methyl-6,7-dihydropyrano[4,3-b]pyrrol-1(4H)-yl)acetic acid (7 mg, 0.014 mmol, 29.6% yield) ¹H NMR (400 MHz, DMSO) δ (ppm): 7.62 (d, 2H), 7.39 (d, 2H), 4.31 (s, 2H), 4.27 (s, 2H), 4.06 (q, 2H), 3.86 (t, 2H), 3.77 (s, 2H), 3.52 (m, 4H), 2.93 (m, 4H), 2.58 (t, 2H), 2.12 (s, 3H), 1.19 (t, 3H)

### Example 4

### Preparation of Sulfur Linked Cycloalkyl Pyrroles

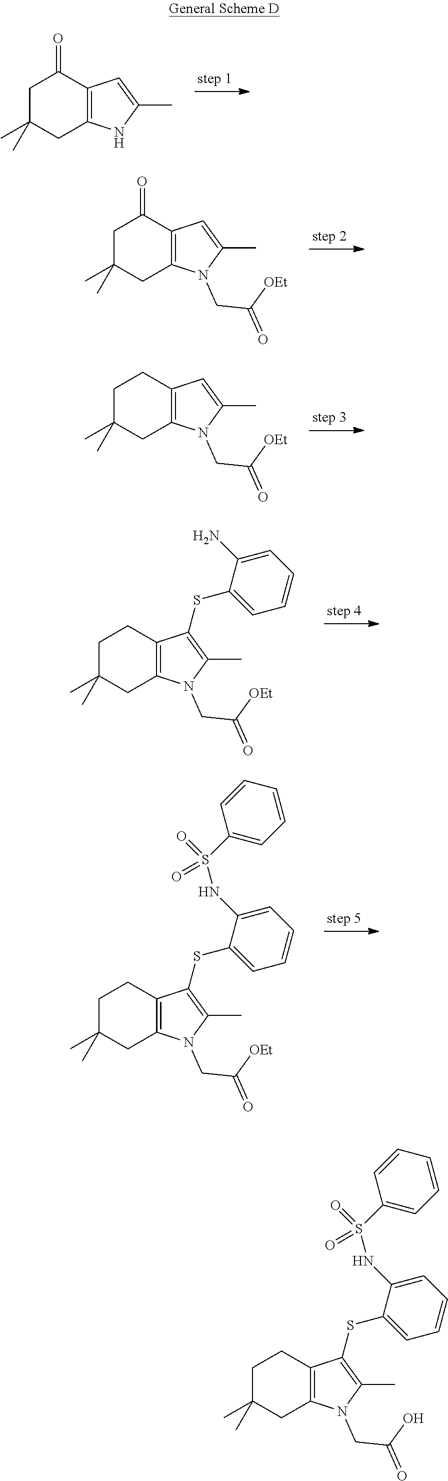

### Preparation of 2-(2,6,6-trimethyl-3-(2-(phenylsulfonamido)phenylthio)-4,5,6,7-tetrahydro-1H-indol-1-yl)acetic acid (I-52)

### Step 1

### Preparation of ethyl 2-(2,6,6-trimethyl-4-oxo-4,5,6,7-tetrahydro-1H-indol-1-yl)acetate

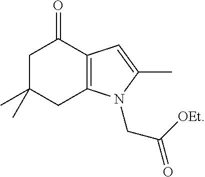 [SMILES: CCOC(=O)Cn1c(C)cc2c1CC(C)(C)CC2=O]

To a stirring solution of 2,6,6-trimethyl-6,7-dihydro-1H-indol-4(51-1)-one (0.243 g, 1.371 mmol) in DMF (Volume: 5 ml) was added ethyl 2-bromoacetate (0.758 ml, 6.85 mmol), potassium iodide (0.068 g, 0.411 mmol) and cesium carbonate (0.893 g, 2.74 mmol). The reaction was capped and stirred for two days at 90° C. The reaction mixture was adsorbed directly onto silica gel loading column using DCM. Purification was obtained by silica gel chromatography using EtOAc and hex as eluents. Product elutes around 25%. Desired fractions were combined, concentrated in vacuo and dried on high vac. NMR shows ethyl 2-(2,6,6-trimethyl-4-oxo-4,5,6,7-tetrahydro-1H-indol-1-yl)acetate (0.161 g, 0.611 mmol, 44.6% yield) desired product as a yellow oil. ¹H NMR (400 MHz, CDCl₃) δ (ppm): 6.28 (s, 1H), 4.49 (s, 2H), 4.23 (q, 2H), 2.52 (s, 2H), 2.33 (s, 2H), 2.17 (s, 3H), 1.28 (t, 3H), 1.10 (s, 6H).

### Step 2

### Preparation of ethyl 2-(2,6,6-trimethyl-4,5,6,7-tetrahydro-1H-indol-1-yl)acetate

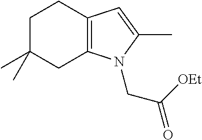 [SMILES: CCOC(=O)Cn1c(C)cc2c1CC(C)(C)CC2]

To a stirring solution of ethyl 2-(2,6,6-trimethyl-4-oxo-4,5,6,7-tetrahydro-1H-indol-1-yl)acetate (0.161 g, 0.611 mmol) in THF (Volume: 5 ml) was added borane-THF complex (1.345 ml, 1.345 mmol) and stirred at rt. The reaction was stirred overnight. Ethanol was added to quench the reaction and the reaction mixture was concentrated in vacuo. The crude mixture was taken up in DCM and purified directly by silica gel chromatography using EtOAc and hex. Purification led to desired product ethyl 2-(2,6,6-trimethyl-4,5,6,7-tetrahydro-1H-indol-1-yl)acetate (0.092 g, 0.369 mmol, 60.3% yield) as a slightly yellow viscous oil. ¹H NMR (400 MHz, CDCl₃) δ (ppm); 5.72 (s, 1H), 4.41 (s, 2H), 4.21 (q, 2H), 2.45 (t, 2H), 2.19 (s, 2H), 2.16 (s, 3H), 1.47 (t, 2H), 1.27 (t, 3H), 0.99 (s, 6H).

### Step 3

### Preparation of ethyl 2-(3-(2-aminophenylthio)-2,6,6-trimethyl-4,5,6,7-tetrahydro-1H-indol-1-yl)acetate

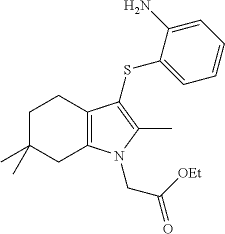 [SMILES: CCOC(=O)Cn1c(C)c(Sc2ccccc2N)c2c1CC(C)(C)CC2]

To a stirring solution of ethyl 2-(2,6,6-trimethyl-4,5,6,7-tetrahydro-1H-indol-1-yl)acetate (0.043 g, 0.172 mmol) in a one to one mixture of DMF (Ratio: 1.000, Volume: 1 ml) and Water (Ratio: 1.000, Volume: 1.000 ml) was added 2-aminobenzenethiol (0.018 ml, 0.172 mmol). This mixture was stirred. In a separate vial potassium iodide (0.143 g, 0.862 mmol) and iodine (0.044 g, 0.172 mmol) were added and dissolved in 1 ml of water. This dark purple solution was added dropwise to the yellow reaction mixture. Upon addition the purple color instantly disappeared and the yellow color intensified. The reaction was stirred for 3 days. At this time, the temperature was raised to 60° C. The reaction was quenched with sodium bicarbonate aqueous and diluted with ethyl acetate. The aqueous layer was extracted 3× with EtOAc and the combined organic layers were washed with brine 3× and dried over sodium sulfate and concentrated in vacuo. The crude mixture was yellow/orange and directly purified by silica gel chromatography. Most products including the desired product eluted at 20% EtOAc and hexane. Ethyl 2-(3-(2-aminophenylthio)-2,6,6-trimethyl-4,5,6,7-tetrahydro-1H-indol-1-yl)acetate material was taken to the next step without any further characterization or purification.

### Step 4

### Preparation of ethyl 2-(2,6,6-trimethyl-3-(2-(phenylsulfonamido)phenylthio)-4,5,6,7-tetrahydro-1H-indol-1-yl)acetate (I-56)

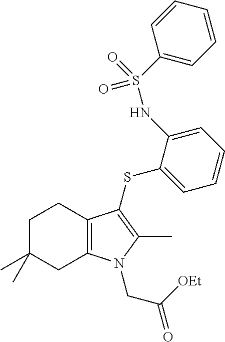 [SMILES: CCOC(=O)Cn1c(C)c(Sc2ccccc2NS(=O)(=O)c2ccccc2)c2c1CC(C)(C)CC2]

To a stirring solution of ethyl 2-(3-(2-aminophenylthio)-2,6,6-trimethyl-4,5,6,7-tetrahydro-1H-indol-1-yl)acetate (0.064 g, 0.172 mmol) in DCE (Volume: 3 ml) was added pyridine (0.069 ml, 0.859 mmol) and benzenesulfonyl chloride (0.022 ml, 0.172 mmol). After 30 minutes at rt the reaction was complete. Reaction mixture was adsorbed directly onto silica gel loading column and purified by silica gel chromatography using EtOAc/hex. Desired fractions were collected and concentrated in vacuo. After drying on under vacuo, NMR showed ethyl 2-(2,6,6-trimethyl-3-(2-(phenylsulfonamido)phenylthio)-4,5,6,7-tetrahydro-1H-indol-1-yl)acetate (0.083 g, 0.162 mmol, 94% yield) as a yellow oil. ¹H NMR (400 MHz, CDCl₃) δ (ppm); 7.75-7.77 (m, 2H), 7.49-7.54 (m, 2H), 7.38-7.42 (m, 2H), 7.04-7.10 (m, 2H), 6.92 (dt, 1H), 6.81 (dd, 1H), 4.44 (s, 2H), 4.20 (q, 2H), 2.15 (s, 2H), 2.08 (s, 3H), 2.02-2.04 (m, 2H), 1.37 (t, 2H), 1.26 (t, 3H), 0.93 (s, 6H).

### Step 5

### Preparation of 2-(2,6,6-trimethyl-3-(2-(phenylsulfonamido)phenylthio)-4,5,6,7-tetrahydro-1,1-indol-1-yl)acetic acid (I-52)

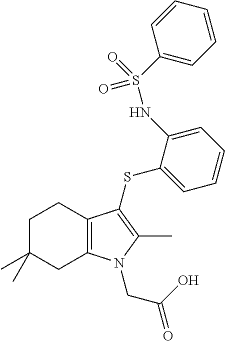 [SMILES: Cc1c(Sc2ccccc2NS(=O)(=O)c2ccccc2)c2c(n1CC(=O)O)CC(C)(C)CC2]

To a stirring solution of ethyl 2-(2,6,6-trimethyl-3-(2-(phenylsulfonamido)phenylthio)-4,5,6,7-tetrahydro-1H-indol-1-yl)acetate (0.043 g, 0.084 mmol) in THF (1 mL) and water 1 mL) was added sodium hydroxide (0.168 ml, 0.168 mmol). The reaction was stirred overnight. The reaction was quenched with 80 μl of 3N HCl and diluted with ethyl acetate. This mixture was split into two layers in a separatory funnel. The aqueous layer was extracted 3× with EtOAc and the combined organic layers were washed once with brine. After drying over sodium sulfate the mixture was concentrated and dried under vacuo for 30 minutes. NMR shows 2-(2,6,6-trimethyl-3-(2-(phenylsulfonamido)phenylthio)-4,5,6,7-tetrahydro-1H-indol-1-yl)acetic acid which is an off-white solid.

### Further Functionalization

### Preparation of ethyl 2-(2,6,6-trimethyl-3-(2-(N-methylphenylsolfonamido)phenylthio)-4,5,6,7-tetrahydro-1H-indol-1-yl)acetate (I-52)

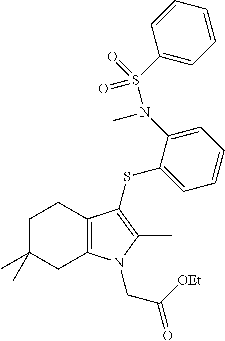 [SMILES: CCOC(=O)Cn1c(C)c(Sc2ccccc2N(C)S(=O)(=O)c2ccccc2)c2c1CC(C)(C)CC2]

To a stirring solution of ethyl 2-(2,6,6-trimethyl-3-(2-(phenylsulfonamido) phenylthio)-4,5,6,7-tetrahydro-1H-indol-1-yl)acetate (0.047 g, 0.092 mmol) in acetonitrile (Volume: 2 ml): was added methyl iodide (0.023 ml, 0.367 mmol) and potassium carbonate (0.051 g, 0.367 mmol). The reaction was heated at 80° C. and stirred for 2 h. Reaction mixture purified directly by silica gel chromatography using hex/EtOAc. After concentration the residue was dried under vacuo to afford a viscous clear oil that looks like it wanted to solidify. The residue was used directly in the next step.

### Preparation of 2-(2,6,6-trimethyl-3-(2-(N-methylphenylsulfonamido) phenylthio)-4,5,6,7-tetrahydro-1H-indol-1-yl)acetic acid (I-53)

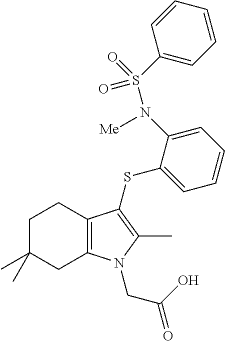 [SMILES: Cc1c(Sc2ccccc2N(C)S(=O)(=O)c2ccccc2)c2c(n1CC(=O)O)CC(C)(C)CC2]

To a stirring solution of ethyl 2-(2,6,6-trimethyl-3-(2-(N-methylphenylsulfonamido) phenylthio)-4,5,6,7-tetrahydro-1H-indol-1-yl)acetate (0.038 g, 0.072 mmol) in THF (Ratio: 1.000, Volume: 1 ml) and Water (Ratio: 1.000, Volume: 1.000 ml) were added sodium hydroxide (0.144 ml, 0.144 mmol). The reaction was stirred overnight. The reaction was quenched with 3N HCl and diluted with brine and EtOAc. The layers were split in an extraction funnel. The aqueous layer was extracted 3× with EtOAc. The combined organic layers were washed once with brine, dried over sodium sulfate and concentrated in vacuo. The brown oil/solid was dried overnight under vacuo. This crude material was then purified by silica gel chromatography. NMR shows 2-(2,6,6-trimethyl-3-(2-(N-methylphenylsulfonamido) phenylthio)-4,5,6,7-tetrahydro-1H-indol-1-yl)acetic acid (0.026 g, 0.052 mmol, 72.3% yield) as a brown solid. ¹H NMR (400 MHz, CDCl₃) 6 (ppm); 7.93 (d, 2H), 7.59 (t, 1H), 7.51 (t, 2H), 6.94-7.09 (m, 3H), 6.62 (dd, 1H), 4.56 (s, 2H), 3.24 (s; 3H), 2.23 (s, 2H), 2.16 (s, 3H), 1.41-1.43 (m, 2H), 1.25 (s, 2H), 0.97 (s, 6H).

### Example 5

### Preparation of Fused Lactame Pyrroles

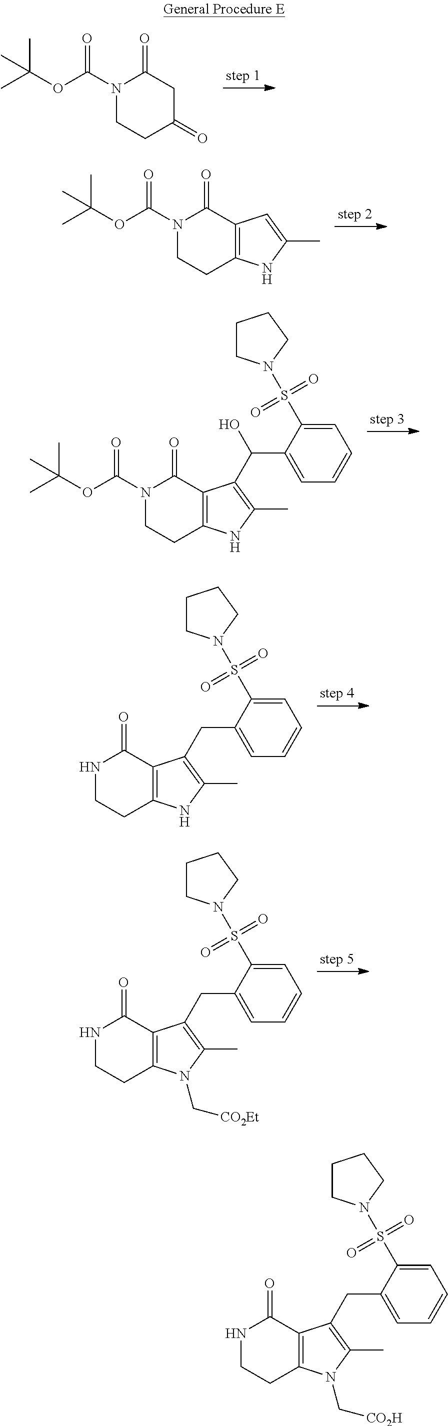

### Preparation of 2-(2-methyl-4-oxo-3-(2-(pyrrolidin-1-ylsulfonyl)benzyl)-4,5,6,7-tetrahydro-1H-pyrrolo[3,2-c]pyridin-1-yl)acetic acid (I-39)

### Step 1

### tert-Butyl ethyl-4-oxo-6,7-dihydro-1H-pyrrolo[3,2-c]pyridine-5(4H)-carboxylate

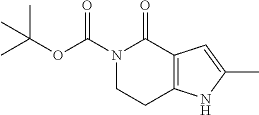 [SMILES: Cc1cc2c([nH]1)CCN(C(=O)OC(C)(C)C)C2=O]

tert-Butyl 2,4-dioxopiperidine-1-carboxylate (0.5 g, 2.345 mmol) was dissolved in EtOH (Volume: 120 ml) and charged with ammonium acetate (0.560 g, 7.27 mmol). The solution was stirred for 5 minutes at rt. To this mixture, 1-chloropropan-2-one (0.198 ml, 2.462 mmol) was added and the reaction was stirred at rt for 14 h. At this time, the reaction was concentrated and diluted with 200 mL EtOAc. It was then extracted with 0.5 M NaOH. This aqueous portion was then re-extracted with EtOAc (100 mL), and the organic layers were combined, dried, filtered, and concentrated. The crude material was purified via SiO2 chromatography using a 0-100% EtOAc/hexane gradient to afford tert-butyl 2-methyl-4-oxo-6,7-dihydro-1H-pyrrolo[3,2-c]pyridine-5(4H)-carboxylate in 78% yield.

### Step 2 and Step 3

### 2-Methyl-3-(2-(pyrrolidin-1-ylsulfonyl)benzyl)-6,7-dihydro-1H-pyrrolo[3,2-c]pyridin-4(5H)-one

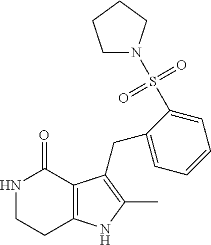 [SMILES: Cc1[nH]c2c(c1Cc1ccccc1S(=O)(=O)N1CCCC1)C(=O)NCC2]

tert-Butyl 2-methyl-4-oxo-6,7-dihydro-1H-pyrrolo[3,2-c]pyridine-5(4H)-carboxylate (0.160 g, 0.639 mmol) was added to a premixed solution of CF₃CH₂OH (Volume: 12 ml) and sodium hydroxide (0.064 ml, 0.639 mmol). 2-(Pyrrolidin-1-ylsulfonyl)benzaldehyde (0.153 g, 0.639 mmol) was then added and the reaction mixture was immediately moved to a hot plate heated at 90° C. After 14 hours, the reaction was carefully quenched with 1N HCl and diluted with DCM (75 mL). It was then transferred to a separatory funnel and washed with NH₄Cl. The organic portion was then dried, filtere, and concentrated. The crude oil was used directly in the next step without any further purification.

tert-Butyl 3-(hydroxy(2-(pyrrolidin-1-ylsulfonyl)phenyl)methyl)-2-methyl-4-oxo-6,7-dihydro-1H-pyrrolo[3,2-c]pyridine-5(4H)-carboxylate (0.313 g, 0.639 mmol) was dissolved in DCM (Volume: 15 ml) and cooled to −78° C. Triethylsilane (0.408 ml, 2.56 mmol) and TFA (0.049 ml, 0.639 mmol) were added consecutively and reaction progress was monitored by LC/MS analysis. After 30 minutes at rt, the reaction was quenched with sodium bicarbonate and extracted 3× with DCM. The organic portions were then combined, dried (Na₂SO₄), filtered, and concentrated. Crude material was purified using 0-20% MeOH/DCM gradiant on SiO2 column to yield 65 mg of 2-Methyl-3-(2-(pyrrolidin-1-ylsulfonyl)benzyl)-6,7-dihydro-1H-pyrrolo[3,2-c]pyridin-4(5H)-one (27%, two steps).

### Step 4

### Ethyl 2-(2-methyl-4-oxo-3-(2-(pyrrolidin-1-ylsulfonyl)benzyl)-4,5,6,7-tetrahydro-1H-pyrrolo[3,2-c]pyridin-1-yl)acetate

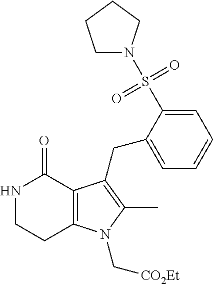 [SMILES: CCOC(=O)Cn1c(C)c(Cc2ccccc2S(=O)(=O)N2CCCC2)c2c1CCNC2=O]

2-Methyl-3-(2-(pyrrolidin-1-ylsulfonyl)benzyl)-6,7-dihydro-1H-pyrrolo[3,2-c]pyridin-4(5H)-one (0.065 g, 0.174 mmol) was dissolved in 2 mL acetonitrile and charged with potassium carbonate (0.072 g, 0.522 mmol). Ethyl bromoacetate (0.019 ml, 0.174 mmol) was then added dropwise. Reaction was heated to 50° C. and stirred overnight. After 14 h, the reaction was transferred to a separatory funnel, diluted with 20 mL DCM and extracted with NH₄Cl. The aqueous was re-extracted with DCM, and then the organic portions were combined, dried, filtered, and concentrated. The crude material was then purified using a 0-100% EtOAc/hexanes gradient to generate 36 mg (45% yield) of Ethyl 2-(2-methyl-4-oxo-3-(2-(pyrrolidin-1-ylsulfonyl)benzyl)-4,5,6,7-tetrahydro-1H-pyrrolo[3,2-c]pyridin-1-yl)acetate.

### Step 5

### 2-(2-Methyl-4-oxo-3-(2-(pyrrolidin-1-ylsulfonyl)benzyl)-4,5,6,7-tetrahydro-1H-pyrrolo[3,2-c]pyridin-1-yl)acetic acid (I-39)

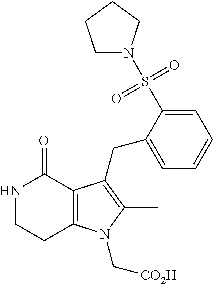 [SMILES: Cc1c(Cc2ccccc2S(=O)(=O)N2CCCC2)c2c(n1CC(=O)O)CCNC2=O]

Ethyl 2-(2-methyl-4-oxo-3-(2-(pyrrolidin-1-ylsulfonyl)benzyl)-4,5,6,7-tetrahydro-1H-pyrrolo[3,2-c]pyridin-1-yl)acetate (0.036 g, 0.078 mmol) was dissolved in a 3:1:1 mixture of THF/MeOH/water and charged with LiOH (9.38 mg, 0.392 mmol). The reaction was stirred at rt for 1 h, at which time LC/MS showed the reaction was complete. Reaction was acidified with 3 N HCl. Mixture was concentrated to near dryness (water left) and was diluted with 3 mL brine solution. This aqueous mixture was then extracted with DCM (3×). The DCM layers were combined, dried, filtered, and concentrated to yield 32 mg of desired product (95%). ¹H NMR (400 MHz, CDCl₃) δ (ppm): 7.98 (dd, 1H), 7.68 (bs, 1H), 7.33 (ddd, 1H), 7.26-7.22 (m, 1H), 6.98 (d, 1H), 4.50 (s, 2H), 4.47 (s, 2H), 3.58-3.53 (m, 2H), 3.35-3.30 (m, 4H), 2.76 (t, 2H), 1.98 (s, 3H), 1.90-1.85 (m, 4H) ppm.

The following compound was made as described in Example 5 and procedure E

### Compound I-50

### 2-(2-Methyl-3-(2-(morpholinosulfonyl)benzyl)-4-oxo-4,5,6,7-tetrahydro-1H-pyrrolo[3,2-c]pyridin-1-yl)acetic acid (I-50)

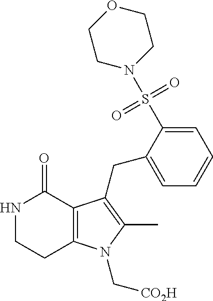 [SMILES: Cc1c(Cc2ccccc2S(=O)(=O)N2CCOCC2)c2c(n1CC(=O)O)CCNC2=O]

¹H NMR (400 MHz, MeOD) δ (ppm): 7.91 (dd, 1H), 7.43 (ddd, 1H), 7.32 (ddd, 1H), 7.09 (d, 1H), 4.71 (s, 2H), 4.54 (s, 2H), 3.74 (t, 4H), 3.53 (t, 2H), 3.20 (t, 4H), 2.83 (t, 2H), 1.97 (s, 3H).

### Example 6

### Synthesis of Sulfonamides

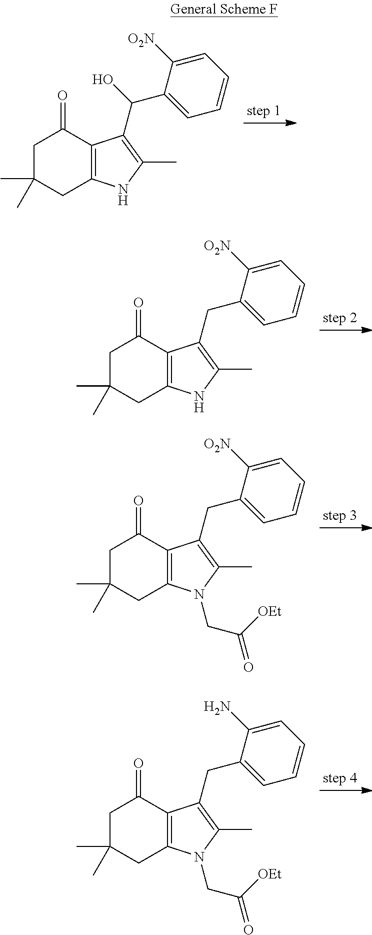

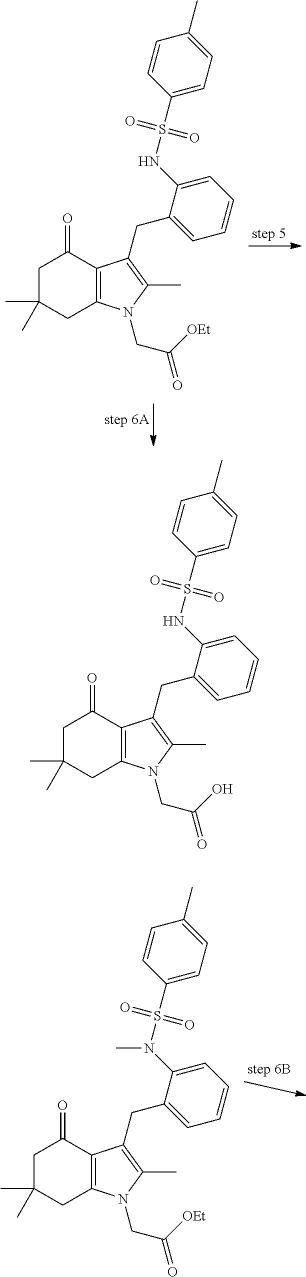

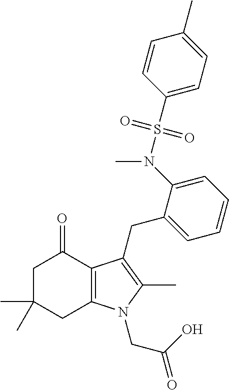 [SMILES: Cc1ccc(S(=O)(=O)N(C)c2ccccc2Cc2c3c(n(CC(=O)O)c2C)CC(C)(C)CC3=O)cc1]

### Preparation of 2-(3-(2-(N,4-dimethylphenylsulfonamido)benzyl)-2,6,6-trimethyl-4-oxo-4,5,6,7-tetrahydro-1H-indol-1-yl)acetic acid (I-33)

### Starting Material Preparation

### 3-(Hydroxy(2-nitrophenyl)methyl)-2,6,6-trimethyl-6,7-dihydro-1H-indol-4(5H)-one

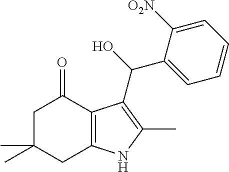 [SMILES: Cc1[nH]c2c(c1C(O)c1ccccc1[N+](=O)[O-])C(=O)CC(C)(C)C2]

2,6,6-Trimethyl-6,7-dihydro-1H-indol-4(5H)-one (6 g, 33.9 mmol) was dissolved in 2,2,2-trifluoroethanol (100 ml) to which was added 2-nitrobenzaldehyde (5.12 g, 33.9 mmol) and 1M NaOH (102 ml, 102 mmol). The reaction mixture was heated to 72° C. for 16 hours increase heat to 92° for 2 hours. The reaction was cooled, water and ice were added, stirred for an hour. The precipitate was filtered to afford 3-(hydroxy(2-nitrophenyl)methyl)-2,6,6-trimethyl-6,7-dihydro-1H-indol-4(5H)-one (6.97 g, 21.23 mmol, 62.7% yield) as a solid

### Step 1

### 2,6,6-Trimethyl-3-(2-nitrobenzyl)-6,7-dihydro-1H-indol-4(5H)-one

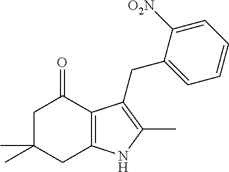 [SMILES: Cc1[nH]c2c(c1Cc1ccccc1[N+](=O)[O-])C(=O)CC(C)(C)C2]

3-(Hydroxy(2-nitrophenyl)methyl)-2,6,6-trimethyl-6,7-dihydro-4H-indol-4(5H)-one (6.97 g, 21.23 mmol) was dissolved in dichloromethane (106 ml) to which was added triethylsilane (6.78 ml, 42.5 mmol). After 5 minutes TFA (9.81 ml, 127 mmol) was added. Reaction heated to 60° C. for 25 minutes. Neutralized with saturated sodium bicarbonate, organics were removed, the ppt filtered 2,6,6-trimethyl-3-(2-nitrobenzyl)-6,7-dihydro-1H-indol-4(5H)-one (6.63 g, 21.23 mmol, 100% yield) was obtained as a solid

### Step 2

### Ethyl 2-(2,6,6-trimethyl-3-(2-nitrobenzyl)-4-oxo-4,5,6,7-tetrahydro-1H-indol-1-yl)acetate

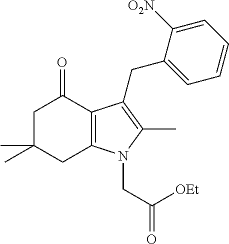 [SMILES: CCOC(=O)Cn1c(C)c(Cc2ccccc2[N+](=O)[O-])c2c1CC(C)(C)CC2=O]

2,6,6-Trimethyl-3-(2-nitrobenzyl)-6,7-dihydro-1H-indol-4(5H)-one (6.63 g, 21.23 mmol) was dissolved in acetonitrile (106 ml) to which was added ethyl 2-bromoacetate (11.74 ml, 106 mmol), potassium iodide (1.762 g, 10.61 mmol), and potassium carbonate (5.87 g, 42.5 mmol). The reaction mixture was heated to 95° C. in a sealed vessel for 18 Hours. Reaction was worked up with dichloromethane and ammonium chloride. Organic layers were combined and dried. Purified by column chromatography afforded ethyl 2-(2,6,6-trimethyl-3-(2-nitrobenzyl)-4-oxo-4,5,6,7-tetrahydro-1H-indol-1-yl)acetate (4.5 g, 11.29 mmol, 53.2% yield).

### Step 3

### Ethyl 2-(3-(2-aminobenzyl)-2,6,6-trimethyl-4-oxo-4,5,6,7-tetrahydro-1H-indol-1-yl)acetate

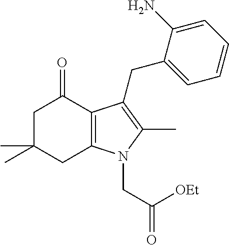 [SMILES: CCOC(=O)Cn1c(C)c(Cc2ccccc2N)c2c1CC(C)(C)CC2=O]

Ethyl 2-(2,6,6-trimethyl-3-(2-nitrobenzyl)-4-oxo-4,5,6,7-tetrahydro-1H-indol-1-yl)acetate (4.12 g, 10.34 mmol) was dissolved in ethanol (73.9 ml), THF (36.9 ml) and saturated ammonium chloride (36.9 ml). Iron (3.46 g, 62.0 mmol) was added. Reaction heated to 70° C. for 2 hours. Reaction mixture was cooled, filtered over celite, washed through the celite with ethyl acetate. The ethyl acetate layer was dried, and was purify by column chromatography to afford the product, ethyl 2-(3-(2-aminobenzyl)-2,6,6-trimethyl-4-oxo-4,5,6,7-tetrahydro-1H-indol-1-yl)acetate (2.2 g, 5.97 mmol, 57.7% yield)

### Step 4

### Ethyl 2-(2,6,6-trimethyl-3-(2-(4-methylphenylsulfonamido)benzyl)-4-oxo-4,5,6,7-tetrahydro-1H-indol-1-yl)acetate

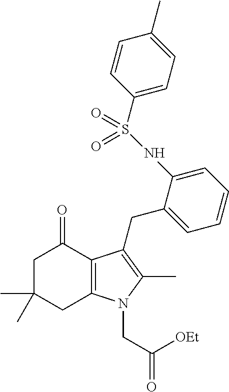 [SMILES: CCOC(=O)Cn1c(C)c(Cc2ccccc2NS(=O)(=O)c2ccc(C)cc2)c2c1CC(C)(C)CC2=O]

Ethyl 2-(3-(2-aminobenzyl)-2,6,6-trimethyl-4-oxo-4,5,6,7-tetrahydro-1H-indol-1-yl)acetate (0.137 g, 0.372 mmol) was dissolved in DCM (3.72 ml) to this was added pyridine (0.150 ml, 1.859 mmol) and then p-toluenesulfonyl chloride (0.071 g, 0.372 mmol). Reaction left stirring at room temperature for 10 minutes, water was added, extract with DCM, The DCM layer was washed with a 0.5M aq. HCl solution. The organic layers were dried and the product was used without further purification. (0.1866 g, 0.357 mmol, 96% yield)

### Step 5

### Ethyl 2-(3-(2-(N,4-dimethylphenylsulfonamido)benzyl)-2,6,6-trimethyl-4-oxo-4,5,6,7-tetrahydro-1H-indol-1-yl)acetate

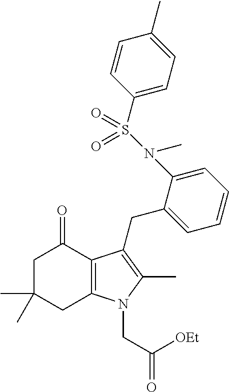 [SMILES: CCOC(=O)Cn1c(C)c(Cc2ccccc2N(C)S(=O)(=O)c2ccc(C)cc2)c2c1CC(C)(C)CC2=O]

Ethyl 2-(2,6,6-trimethyl-3-(2-(4-methylphenylsulfonamido)benzyl)-4-oxo-4,5,6,7-tetrahydro-1H-indol-1-yl)acetate (0.0837 g, 0.160 mmol) was dissolved in acetonitrile (2 ml) to this was added potassium carbonate (0.044 g, 0.320 mmol) and then methyl iodide (0.011 ml, 0.176 mmol). Reaction heated to 70° C. for 16 hours., 0.5 eq additional methyl iodide was added to the reaction mixture, heated to 80° C. for 2 hours. Reaction was cooled, washed with ammonium chloride and dichloromethane. DCM layer, extracted, dried, purified by column chromatography to afford ethyl 2-(3-(2-(N,4-dimethylphenylsulfonamido)benzyl)-2,6,6-trimethyl-4-oxo-4,5,6,7-tetrahydro-1H-indol-1-yl)acetate (0.049 g, 0.091 mmol, 57.0% yield)

### Step 6A

### 2-(2,6,6-Trimethyl-3-(2-(4-methylphenylsulfonamido)benzyl)-4-oxo-4,5,6,7-tetrahydro-1H-indol-1-yl)acetic acid (I-23)

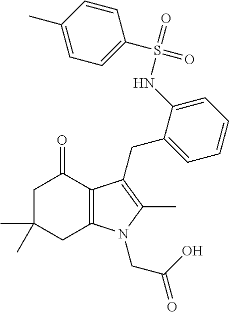 [SMILES: Cc1ccc(S(=O)(=O)Nc2ccccc2Cc2c3c(n(CC(=O)O)c2C)CC(C)(C)CC3=O)cc1]

Ethyl 2-(2,6,6-trimethyl-3-(2-(4-methylphenylsulfonamido)benzyl)-4-oxo-4,5,6,7-tetrahydro-1H-indol-1-yl)acetate was saponified using the procedure as in 2-(3-(2-(N,4-dimethylphenylsulfonamido)benzyl)-2,6,6-trimethyl-4-oxo-4,5,6,7-tetrahydro-1H-indol-1-yl)acetic acid to afford 2-(2,6,6-trimethyl-3-(2-(4-methylphenylsulfonamido)benzyl)-4-oxo-4,5,6,7-tetrahydro-1H-indol-1-yl)acetic acid as a clean product without further purification. ¹H NMR (400 MHz, CDCl₃) δ (ppm): 9.51 (s, 1H), 7.66 (d, 2H), 7.43 (d, 1H), 7.19 (d, 2H), 7.00-7.09 (m, 3H), 4.50 (s, 2H), 3.56 (s, 2H), 2.48 (s, 2H), 2.37 (s, 3H), 2.36 (s, 2H), 2.16 (s, 3H) 1.07 (s, 6H)

### Step 6B

### 2-(3-(2-(N,4-Dimethylphenylsulfonamido)benzyl)-2,6,6-trimethyl-4-Oxa-4,5,6,7-tetrahydro-1H-indol-1-0)acetic acid (I-33)

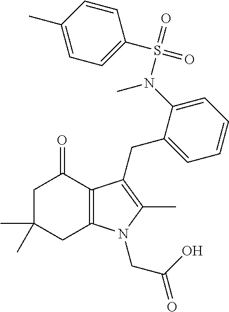 [SMILES: Cc1ccc(S(=O)(=O)N(C)c2ccccc2Cc2c3c(n(CC(=O)O)c2C)CC(C)(C)CC3=O)cc1]

Ethyl 2-(3-(2-(N,4-dimethylphenylsulfonamido)benzyl)-2,6,6-trimethyl-4-oxo-4,5,6,7-tetrahydro-1H-indol-1-yl)acetate (0.035 g, 0.065 mmol) was dissolved in THF (Volume: 2 ml) to this was added water (2 ml) and 1M NaOH (0.065 ml, 0.196 mmol). Reaction heated to 40° C. for 30 minutes. Reaction cooled, neutralized with 1M HCl solution. Organics were removed and the precipitate was filtered. 2-(3-(2-(N,4-dimethylphenylsulfonamido)benzyl)-2,6,6-trimethyl-4-oxo-4,5,6,7-tetrahydro-1H-indol-1-yl)acetic acid (11.8 mg, 0.023 mmol, 35.6% yield)

¹H NMR (400 MHz, CDCl₃) δ (ppm): 7.65 (d, 2H), 7.31 (d, 2H), 7.08 (d, 1H), 6.91-6.98 (m, 2H), 6.53 (d, 1H), 4.59 (s, 2H), 4.00 (d, 2H), 3.27 (s, 3H), 2.54 (s, 2H), 2.46 (s, 3H), 2.23 (d, 2H), 2.14 (s, 3H), 1.08 (d, 6H)

The following compounds were prepared analogously to those described in Experiment 6 and General Scheme F

### Compound I-22

### 2-(3-(2-(2,4-Difluorophenylsulfonamido)benzyl)-2,6,6-trimethyl-4-oxo-4,5,6,7-tetrahydro-1H-indol-1-yl)acetic acid (I-22)

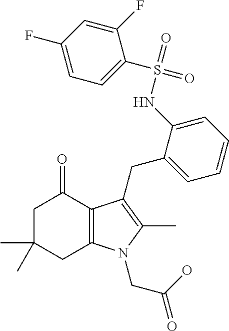 [SMILES: Cc1c(Cc2ccccc2NS(=O)(=O)c2ccc(F)cc2F)c2c(n1CC(=O)O)CC(C)(C)CC2=O]

¹H NMR (400 MHz, CDCl₃) δ (ppm): 9.68 (s, 1H), 7.78 (d, 1H), 7.38-7.51 (m, 3H), 6.98-7.12 (m, 3H), 4.44 (s, 2H), 4.20-4.25 (m, 2H), 2.45 (s, 2H), 2.36 (s, 2H), 2.13 (s, 3H), 1.07 (s, 6H)

### Compound I-24

### 2-(2,6,6-Trimethyl-4-oxo-3-(2-(phenylsulfonamido)benzyl)-4,5,6,7-tetrahydro-1H-indol-1-yl)acetic acid (I-24)

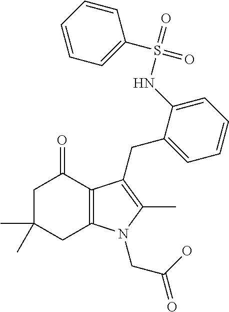 [SMILES: Cc1c(Cc2ccccc2NS(=O)(=O)c2ccccc2)c2c(n1CC(=O)O)CC(C)(C)CC2=O]

¹H NMR (400 MHz, CDCl₃) δ (ppm): 9.59 (s, 1H), 7.76 (d, 2H), 7.37-7.48 (m, 4H), 7.00-7.11 (m, 3H), 4.50 (s, 2H), 3.52 (s, 2H), 2.47 (s, 2H), 2.35 (s, 2H), 2.15 (s, 3H), 1.06 (s, 6H)

### Compound I-25

### 2-(2,6,6-Trimethyl-4-oxo-3-(2-(4-(trifluoromethyl)phenylsulfonamido)benzyl)-4,5,6,7-tetrahydro-1H-indol-1-yl)acetic acid (I-25)

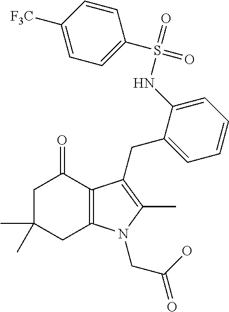 [SMILES: Cc1c(Cc2ccccc2NS(=O)(=O)c2ccc(C(F)(F)F)cc2)c2c(n1CC(=O)O)CC(C)(C)CC2=O]

¹H NMR (400 MHz, CDCl₃) δ (ppm): 9.90 (s, 1H), 7.89 (d, 2H), 7.66 (d, 2H), 7.44 (d, 1H), 7.05-7.12 (m, 3H) 4.51 (s, 2H), 3.76 (3, 2H), 2.47 (s, 2H), 2.36 (s, 2H), 2.17 (d, 3H), 1.06 (s, 6H)

### Compound I-45

### 2-(3-(3-(N,4-Dimethylphenylsulfonamido)benzyl)-2,6,6-trimethyl-4-oxo-4,5,6,7-tetrahydro-1H-indol-1-yl)acetic acid (I-45)

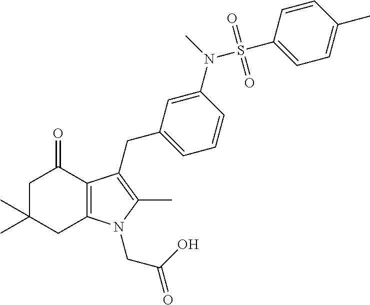

¹H NMR (400 MHz, CDCl₃) δ (ppm): 7.40 (d, 1H), 7.21 (d, 2H), 7.08-7.15 (m, 2H), 6.98 (s, 1H), 6.74 (d, 1H) 4.55 (s, 2H), 4.07 (s, 2H), 3.08 (s, 3H), 2.53 (s, 2H), 2.49 (s, 3H), 2.30 (s, 2H), 2.07 (s, 3H), 1.08 (3, 6H)

### Compound I-47

### 2-(2,6,6-Trimethyl-3-(2-(4-(pyrrolidin-1-yl)phenylsulfonamido)benzyl)-4-oxo-4,5,6,7-tetrahydro-1H-indol-1-yl)acetic acid (I-47)

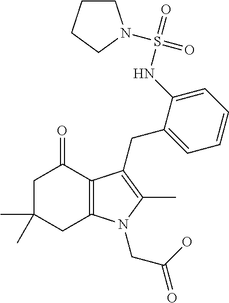 [SMILES: Cc1c(Cc2ccccc2NS(=O)(=O)N2CCCC2)c2c(n1CC(=O)O)CC(C)(C)CC2=O]

¹H NMR (400 MHz, CDCl₃) δ (ppm): 7.08-7.10 (m, 1H), 6.95-6.99 (m, 3H), 4.52, (s, 2H), 4.02 (s, 2H), 3.19-3.22 (m, 4H), 2.31 (s, 2H), 2.30 (s, 2H), 2.09 (s, 3H), 1.69-1.73 (m, 4H), 1.07 (s, 6H)

### Compound I-49

### 2-(2,6,6-Trimethyl-3-(2-(4-morpholinophenylsulfonamido)benzyl)-4-oxo-4,5,6,7-tetrahydro-1H-indol-1-yl)acetic acid (I-49)

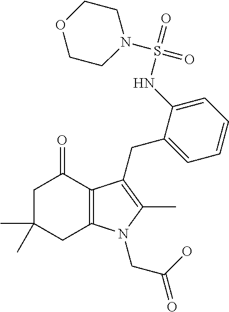 [SMILES: Cc1c(Cc2ccccc2NS(=O)(=O)N2CCOCC2)c2c(n1CC(=O)O)CC(C)(C)CC2=O]

¹H NMR (400 MHz, CDCl₃) δ (ppm): 9.04 (s, 1H), 7.48 (d, 1H), 7.12-7.17 (m, 2H), 7.00-7.03 (m, 1H), 4.53 (s, 2H), 4.09 (s, 2H), 3.63-3.66, (m, 4H), 3.24 (t, 4H), 2.51 (s, 2H), 2.37 (s, 2H), 2.19 (s, 3H), 1.09 (s, 6H)

### Compound I-48

### 2-(3-(2-(4-Hydroxyphenylsulfonamido)benzyl)-2,6,6-trimethyl-4-oxo-4,5,6,7-tetrahydro-1H-indol-1-yl)acetic acid (I-48)

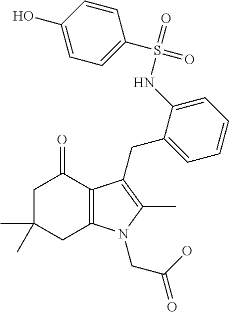 [SMILES: Cc1c(Cc2ccccc2NS(=O)(=O)c2ccc(O)cc2)c2c(n1CC(=O)O)CC(C)(C)CC2=O]

¹H NMR (400 MHz, CDCl₃) δ (ppm): 7.71 (s, 2H), 7.52 (s, 2H), 4.20-4.23 (m, 2H), 4.09-4.14 (m, 2H), 2.34-2.37 (m, 2H), 2.09-2.16 (m, 2H), 2.05 (s, 3H), 1.25 (s, 6H)

### Compound I-27

### 2-(3-(2-(4-Isopropylphenylsulfonamido)benzyl)-2,6,6-trimethyl-4-oxo-4,5,6,7-tetrahydro-1H-indol-1-yl)acetic acid (I-27)

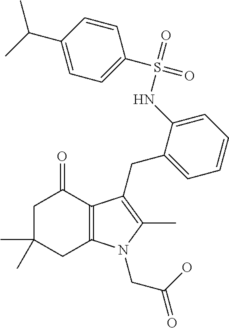 [SMILES: Cc1c(Cc2ccccc2NS(=O)(=O)c2ccc(C(C)C)cc2)c2c(n1CC(=O)O)CC(C)(C)CC2=O]

¹H NMR (400 MHz, CDCl₃) δ (ppm): 9.54 (s, 1H), 7.83 (d, 2H), 7.70 (d, 2H), 7.44 (d, 1H), 7.36-7.7.40 (m, 1H), 6.97-7.04 (m, 2H), 4.46; (s, 2H), 4.34 (s, 2H), 2.89-2.94, (m, 1H), 2.61 (s, 2H), 2.46 (s, 2H), 2.36 (s, 2H), 2.25 (s, 2H), 2.13 (s, 2H), 2.09 (s, 3H), 1.22-1.26 (m, 6H), 1.08 (d, 6H)

### Compound I-34

### 2-(3-(2-(4-Isopropyl-N-methylphenylsulfonamido)benzyl)-2,6,6-trimethyl-4-oxo-4,5,6,7-tetrahydro-1H-indol-1-yl)acetic acid (I-34)

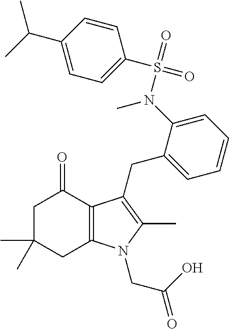 [SMILES: Cc1c(Cc2ccccc2N(C)S(=O)(=O)c2ccc(C(C)C)cc2)c2c(n1CC(=O)O)CC(C)(C)CC2=O]

¹H NMR (400 MHz, CDCl₃) δ (ppm):7.67-7.69 (m, 2H), 7.35 (d, 2H), 7.07-7.10 (m, 1H), 6.91-6.99 (m, 2H), 6.56 (d, 1H), 4.60 (s, 2H), 4.54 (d, 1H), 4.00 (d, 1H), 3.27 (d, 3H), 2.99-3.02 (m, 1H), 2.54 (s, 2H), 2.23 (s, 2H), 2.14 (d, 3H), 1.28-1.31 (m, 6H), 1.08 (d, 6H)

### Compound I-35

### 2-(2,6,6-Trimethyl-3-(2-(N-methylphenylsulfonamido)benzyl)-4-oxo-4,5,6,7-tetrahydro-1H-indol-1-yl)acetic acid (I-35)

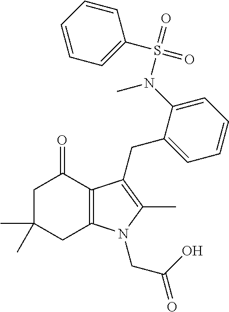 [SMILES: Cc1c(Cc2ccccc2N(C)S(=O)(=O)c2ccccc2)c2c(n1CC(=O)O)CC(C)(C)CC2=O]

¹H NMR (400 MHz, CD₃OD) δ (ppm):7.68-7.74 (m, 3H), 7.58-7.62 (m, 2H), 7.06-7.10 (m, 1H), 6.92-6.95 (m, 2H), 6.45 (d, 1H), 4.72 (s, 2H), 3.94 (d, 1H), 3.28 (d, 3H), 2.63 (s, 2H), 2.20 (d, 2H), 2.10 (d, 3H), 1.08 (d, 6H)

### Compound I-36

### 2-(2,6,6-Trim ethyl-4-oxo-3-(2-(phenylsulfonamido)-5-(2,2,2-trifluoroethoxy)benzyl)-4,5,6,7-tetrahydro-1H-indol-1-yl)acetic acid (I-36)

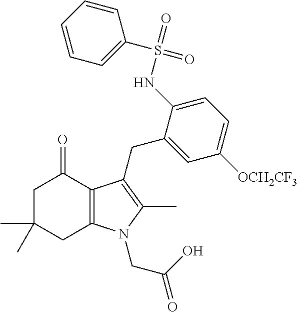

¹H NMR (400 MHz, CDCl₃) δ (ppm): 9.37 (s, 1H), 7.74 (d, 2H), 7.49-7.53 (m, 1H), 7.38-7.43 (m, 2H), 6.66-6.69 (m, 2H), 6.58 (d, 1H), 4.53 (s, 2H), 4.20-4.26 (m, 2H), 3.33 (s, 2H), 2.47 (s, 2H), 2.32 (s, 2H), 2.17 (s, 3H), 1.06 (s, 6H)

### Compound I-40

### 2-(2,6,6-Trimethyl-4-oxo-3-(2-(pyrrolidine-1-sulfonamido)benzyl)-4,5,6,7-tetrahydro-1H-indol-1-yl)acetic acid (I-40)

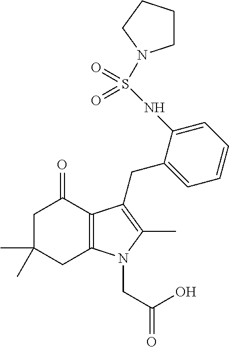 [SMILES: Cc1c(Cc2ccccc2NS(=O)(=O)N2CCCC2)c2c(n1CC(=O)O)CC(C)(C)CC2=O]

¹H NMR (400 MHz, CDCl₃) δ (ppm): 8.91 (s, 1H), 7.48-7.50 (m, 1H), 7.12-7.17 (m, 2H), 6.98-7.00 (m, 1H), 4.48 (s, 2H), 3.32-3.35 (m, 4H), 2.49 (s, 2H), 2.36 (s, 2H), 2.17 (s, 3H), 1.82-1.86 (m, 4H), 1.09 (s, 6H)

### Compound I-43

### 2-(2,6,6-Trimethyl-3-(2-(N-methylpyrrolidine-1-sulfonamido)benzyl)-4-oxo-4,5,6,7-tetrahydro-1H-indol-1-yl)acetic acid (I-43)

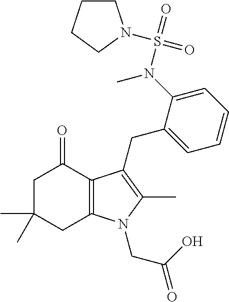 [SMILES: Cc1c(Cc2ccccc2N(C)S(=O)(=O)N2CCCC2)c2c(n1CC(=O)O)CC(C)(C)CC2=O]

¹H NMR (400 MHz, CDCl₃) δ (ppm): 7.39-7.41 (m, 1H), 7.09-7.17 (m, 2H), 6.80 (d, 1H), 4.60 (s, 2H), 3.45 (s, 4H), 3.28 (s, 3H), 3.26-3.27 (m, 2H), 2.37 (s, 2H), 2.08 (s, 3H), 1.94-1.98 (m, 4H), 1.27 (d, 3H), 1.10 (s, 3H).

### Compound I-51

### 2-(2,6,6-Trimethyl-3-(3-(N-methylpyrrolidine-1-sulfonamido)benzyl)-4-oxo-4,5,6,7-tetrahydro-1H-indol-1-yl)acetic acid (I-51)

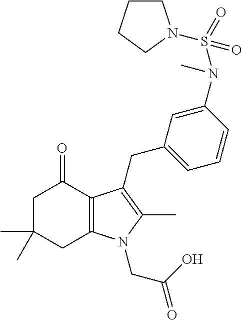 [SMILES: Cc1c(Cc2cccc(N(C)S(=O)(=O)N3CCCC3)c2)c2c(n1CC(=O)O)CC(C)(C)CC2=O]

¹H NMR (400 MHz, CDCl₃) δ (ppm): 7.13-7.19 (m, 4H), 4.56 (s, 2H), 4.11 (s, 2H), 3.17-3.22 (m, 6H), 2.54 (s, 2H), 2.30 (s, 2H), 2.10 (s, 3H), 1.72-1.76 (m, 4H), 1.25 (s, 3H), 1.10 (s, 6H)

### Compound I-46

### 2-(3-(2-(4-(Benzyloxy)phenylsulfonamido)benzyl)-2,6,6-trimethyl-4-oxo-4,5,6,7-tetrahydro-1H-indol-1-yl)acetic acid (I-46)

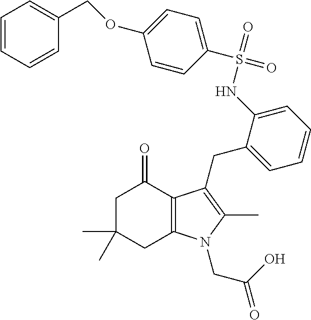 [SMILES: Cc1c(Cc2ccccc2NS(=O)(=O)c2ccc(OCc3ccccc3)cc2)c2c(n1CC(=O)O)CC(C)(C)CC2=O]

¹H NMR (400 MHz, CDCl₃) δ (ppm): 9.40 (s, 1H), 7.71 (d, 2H), 7.34-7.43 (m, 5H), 6.92-7.09 (m, 6H), 5.07 (s, 2H), 4.51 (s, 2H), 3.61 (s, 2H), 2.47 (s, 2H), 2.35 (s, 2H) 2.15 (s, 3H), 1.07 (s, 6H)

### Compound I-38

### 2-(3-(2-(N-Ethyl-4-methylphenylsulfonamido)benzyl)-2,6,6-trimethyl-4-oxo-4,5,6,7-tetrahydro-1H-indol-1-yl)acetic acid (I-38)

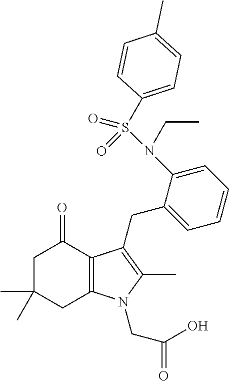 [SMILES: CCN(c1ccccc1Cc1c2c(n(CC(=O)O)c1C)CC(C)(C)CC2=O)S(=O)(=O)c1ccc(C)cc1]

¹H NMR (400 MHz, CDCl₃) δ (ppm): 7.64 (d, 2H), 7.28 (d, 2H), 7.08-7.11 (m, 1H), 6.98-7.04 (m, 1H), 6.80-6.82 (m, 1H), 6.60-6.62 (m, 1H), 4.60 (s, 2H), 4.44-4.48 (d, 2H), 4.08-4.12 (d, 2H), 3.86-3.94 (m, 1H), 3.40-3.50 (m, 1H), 2.57 (s, 2H), 2.44 (s, 3H), 2.27 (s, 2H), 2.06 (s, 3H), 1.17-1.20 (m, 3H), 1.10 (s, 6H)

### Example 7

### Cycloalkyl Pyrroles without a Keto Group

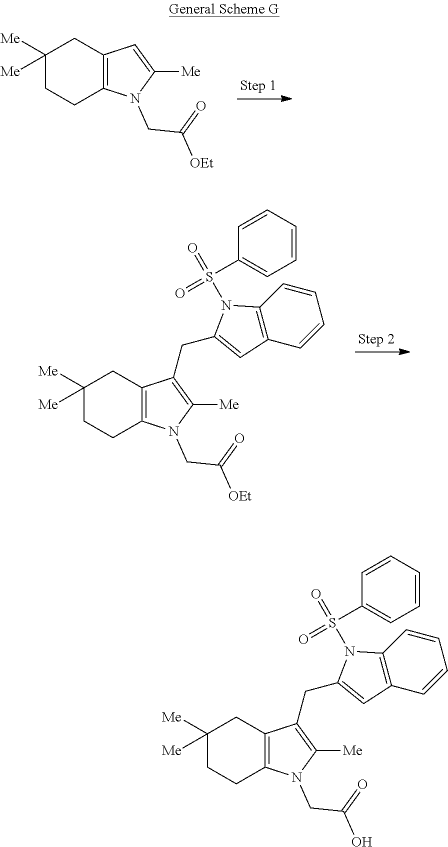 [SMILES: CCOC(=O)Cn1c(C)cc2c1CCC(C)(C)C2 ; CCOC(=O)Cn1c(C)c(Cc2cc3ccccc3n2S(=O)(=O)c2ccccc2)c2c1CCC(C)(C)C2 ; Cc1c(Cc2cc3ccccc3n2S(=O)(=O)c2ccccc2)c2c(n1CC(=O)O)CCC(C)(C)C2]

### Preparation of 2-(2,5,5-trimethyl-3-41-(phenylsulfonyl)-1H-indol-2-yl)methyl)-4,5,6,7-tetrahydro-1H-indol-1-yl)acetic acid (I-1)

### Step 1

### 2-(2,5,5-Trimethyl-34(1-(phenylsulfonyl)-1H-indol-2-yl)methyl)-4,5,6,7-tetrahydro-1H-indol-1-yl)acetic acid ethyl ester (I-57)

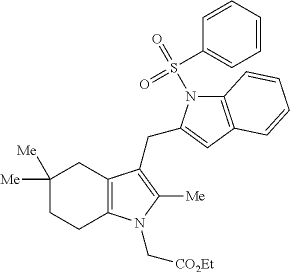 [SMILES: CCOC(=O)Cn1c(C)c(Cc2cc3ccccc3n2S(=O)(=O)c2ccccc2)c2c1CCC(C)(C)C2]

To a 0° C. solution of trimethylsilyl trifluoromethanesulfonate (0.135 ml, 0.746 mmol) in dichloromethane (2 mL) was added a solution of ethyl 2-(2,5,5-trimethyl-4,5,6,7-tetrahydro-1H-indol-1-yl)acetate (93.0 mg, 0.373 mmol) and 1-(phenylsulfonyl)-1H-indole-2-carbaldehyde (106 mg, 0.373 mmol) in dichloromethane (5 ml). The reaction was stirred at 0° C. for 15 minutes after which neat triethylsilane (0.238 ml, 1.49 mmol) was added. The reaction was slowly warmed to room temperature and allowed to stir for 12 hours, after which the reaction was quenched with water, extracted with dichloromethane (3×30 mL), dried (sodium sulfate), filtered and concentrated to an orange residue. Purification was achieved by silica gel chromatography (Luknova 40 g, 20 ml/min) using 10 to 70% ethyl acetate (containing 1% triethylamine) in hexanes over 60 minutes. The product, ethyl 2-(2,5,5-trimethyl-34(1-(phenylsulfonyl)-1H-indol-2-yl)methyl)-4,5,6,7-tetrahydro-1H-indol-1-yl)acetate (86.2 mg, 0.166 mmol, 45% yield) was isolated as a pink foam. ¹H (400 MHz, CDCl₃) δ (ppm): 8.24 (d, 1H), 7.79 (m, 2H), 7.54 (t, 1H), 7.43 (app t., 2H), 7.33 (d, 1H), 7.25 (m, 1H), 7.19 (m, 1H), 5.94 (s, 1H), 4.47 (s, 2H), 4.22 (q, 2H), 3.94 (s, 2H), 2.43 (t, 2H), 1.94 (s, 3H), 1.81 (s, 2H), 1.52 (t, 2H), 1.29 (t, 3H), 0.85 (s, 6H).

### Step 2

### 2-(2,5,5-Trimethyl-3-((1-(phenylsulfonyl)-1H-indol-2-yl)methyl)-4,5,6,7-tetrahydro-4H-indol-1-yl)acetic acid (I-1)

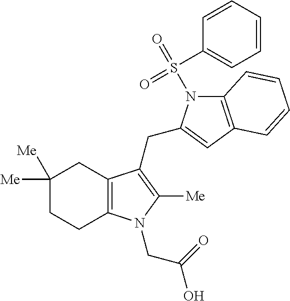 [SMILES: Cc1c(Cc2cc3ccccc3n2S(=O)(=O)c2ccccc2)c2c(n1CC(=O)O)CCC(C)(C)C2]

To a solution of ethyl 2-(2,5,5-trimethyl-3-((1-(phenylsulfonyl)-1H-indol-2-yl)methyl)-4,5,6,7-tetrahydro-1H-indol-1-yl)acetate (86.2 mg, 0.166 mmol) in tetrahydrofuran (2 mL) and water (2 mL) was added an aqueous 1M solution of sodium hydroxide (0.183 mL, 0.183 mmol). The reaction was stirred at room temperature for 12 hours, after which aqueous 3M hydrochloric acid solution was added (0.061 mL). After removal of the tetrahydrofuran, the reaction mixture was purified by reverse phase HPLC using 10 to 90% acetonitrile in water (spiked w/0.025% TFA) over 45 minutes. The product, 2-(2,5,5-trimethyl-3-((1-(phenylsulfonyl)-1H-indol-2-yl)methyl)-4,5,6,7-tetrahydro-1H-indol-1-yl)acetic acid (26.2 mg, 0.0530 mmol, 32% yield) was isolated as an off-white solid. ¹H NMR (400 MHz, CDCl₃) δ (ppm): 8.25 (d, 1H), 7.79 (m, 2H), 7.53 (t, 1H), 7.42 (app t., 2H), 7.33 (d, 1H), 7.25 (m, 1H), 7.19 (m, 1H), 5.93 (s, 1H), 4.54 (s, 2H), 3.94 (s, 2H), 2.43 (t, 2H), 1.96 (s, 3H), 1.81 (s, 2H), 1.53 (t, 2H), 0.85 (s, 6H).

The following compound was made as described in Example 7 and General Scheme G

### Compound I-37

### 2-(2,6,6-Trimethyl-3-01-(morpholinosulfonyl)-1H-indol-2-yl)methyl)-4-oxo-4,5,6,7-tetrahydro-1H-indol-1-yl)acetic acid (I-37)

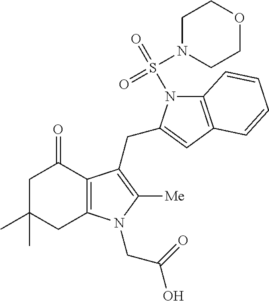 [SMILES: Cc1c(Cc2cc3ccccc3n2S(=O)(=O)N2CCOCC2)c2c(n1CC(=O)O)CC(C)(C)CC2=O]

¹H NMR (400 MHz, CD₃OD) δ (ppm): (d, J=6.4 Hz, 1H), 7.33-7.30 (m, 1H), 7.20-7.10 (m, 2H), 5.84 (s, 1H), 4.74 (s, 2H), 4.33 (s, 2H), 3.64-3.62 (m, 4H), 4.92-4.80 (m, 4H, under DMeOD peak), 2.68 (s, 2H), 2.27 (s, 2H), 2.06 (s, 3H), 1.12 (s, 6H).

### Example 8

### Synthesis of Oximes

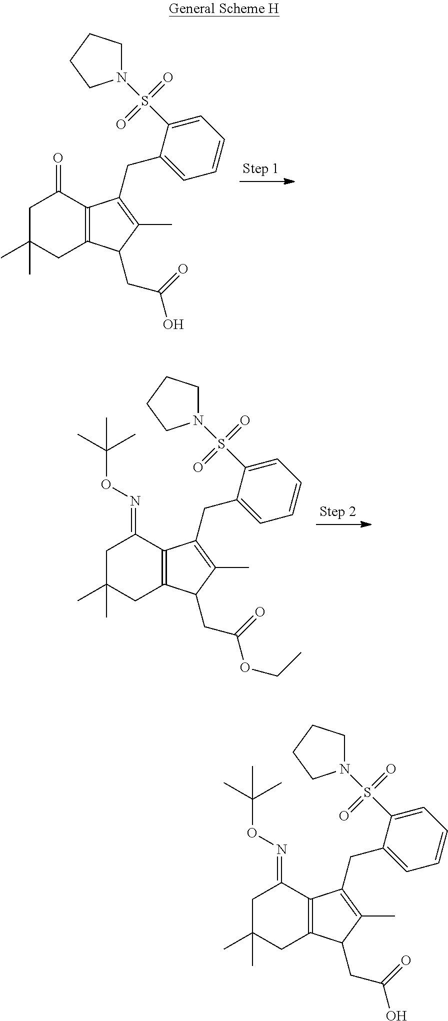 [SMILES: CC1=C(Cc2ccccc2S(=O)(=O)N2CCCC2)C2=C(CC(C)(C)CC2=O)C1CC(=O)O ; CCOC(=O)CC1C(C)=C(Cc2ccccc2S(=O)(=O)N2CCCC2)C2=C1CC(C)(C)C/C2=N\OC(C)(C)C ; CC1=C(Cc2ccccc2S(=O)(=O)N2CCCC2)C2=C(CC(C)(C)C/C2=N\OC(C)(C)C)C1CC(=O)O]

### Step 1 and Step 2

### (E)-2-(4-(tert-Butoxyimino)-2,6,6-trimethyl-3-(2-(pyrrolidin-1-ylsulfonyl)benzyl)-4,5,6,7-tetrahydro-M-indol-1-yl)acetic acid (I-30)

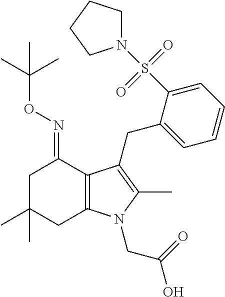 [SMILES: Cc1c(Cc2ccccc2S(=O)(=O)N2CCCC2)c2c(n1CC(=O)O)CC(C)(C)C/C2=N\OC(C)(C)C]

A mixture of 2-(2,6,6-trimethyl-4-oxo-3-(2-(pyrrolidin-1-ylsulfonyl)benzyl)-4,5,6,7-tetrahydro-1H-indol-1-yl)acetic acid (200 mg, 0.43 mmol) and O-t-butylhydroxylamine hydrochloride (117 mg, 1.31 mmol) in absolute ethanol (2 mL) was prepared at room temperature. One drop of concentrated hydrochloric acid(aq) was added and the stirred mixture was heated overnight at 60° C. The solution was cooled to room temperature and diluted with ethyl acetate (60 mL), then washed with 2×10 mL water, dried over Na₂SO₄, filtered and concentrated by rotary evaporation. The major isomer was collected as a colorless glass (120 mg) by chromatography over SiO₂, eluting with hexane/ethyl acetate. The product was used directly in the next reaction without characterization. The colorless glass (120 mg, 0.21 mmol) was mixed in tetrahydrofuran/water (4 mL, 1:1) at room temperature. A solution of 1N NaOH(aq) (0.5 mL, 0.5 mmol) was added and the mixture was stirred for 1 hr at room temperature. The mixture was diluted with 0.36 M NaH₂PO₄(aq) (10 mL) and ethyl acetate (50 mL), the layers were well mixed and separated. The organic phase was washed with water (2×10 mL) and brine, then dried over Na₂SO₄, filtered and concentrated by rotary evaporation. (E)-2-(4-(tert-Butoxyimino)-2,6,6-trimethyl-3-(2-(pyrrolidin-1-ylsulfonyl)benzyl)-4,5,6,7-tetrahydro-1H-indol-1-yl)acetic acid (96 mg) was obtained as a white foam after chromatography over SiO₂ with hexane/ethyl acetate as eluant. ¹H-NMR (CDCl₃): δ (ppm): 7.95 (dd, 1H), 7.35 (dt, 1H), 7.19 (t, 1H), 7.16 (d, 1H), 4.57 (s, 2H), 4.53 (s, 2H), 3.3-3.4 (m, 4H), 2.51 (s, 2H), 2.40 (s, 2H), 1.95 (s, 3H), 1.9-2.0 (m, 4H), 1.09 (s, 6H), 0.95, (s, 9H) ppm.

The following compounds were made analogously to Example 8 and General Scheme H

### Compound I-28

### (E)-2-(4-(Methoxyimino)-2,6,6-trimethyl-3-(2-(pyrrolidin-1-ylsulfonyl)benzyl)-4,5,6,7-tetrahydro-1H-indol-1-yl)acetic acid (I-28)

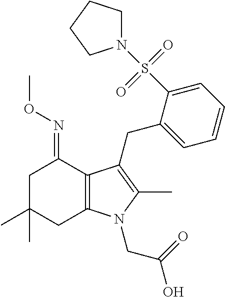 [SMILES: CO/N=C1\CC(C)(C)Cc2c1c(Cc1ccccc1S(=O)(=O)N1CCCC1)c(C)n2CC(=O)O]

¹H-NMR (CDCl₃): δ (ppm): 7.96 (d, 1H), 7.32 (t, 1H), 7.22 (t, 1H), 7.04 (d, 1H), 4.54 (s, 2H), 4.53 (s, 2H), 3.54 (s, 3H), 3.3-3.4 (m, 4H), 2.47 (s, 2H), 2.40 (s, 2H), 1.95 (s, 3H), 1.9-2.0 (m, 4H), 1.07 (s, 6H) ppm.

### Compound I-29

### (E)-2-(4-(Ethoxyimino)-2,6,6-trimethyl-3-(2-(pyrrolidin-1-ylsulfonyl)benzyl)-4,5,6,7-tetrahydro-1H-indol-1-yl)acetic acid (I-29)

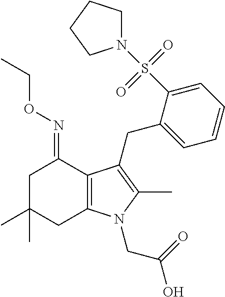 [SMILES: CCO/N=C1\CC(C)(C)Cc2c1c(Cc1ccccc1S(=O)(=O)N1CCCC1)c(C)n2CC(=O)O]

¹H-NMR (CDCl₃): δ (ppm): 7.94 (dd, 1H), 7.31 (dt, 1H), 7.20 (t, 1H), 7.05 (d, 1H), 4.52 (s, 2H), 4.51 (s, 2H), 3.77 (q, 2H), 3.3-3.4 (m, 4H), 2.48 (s, 2H), 2.40 (s, 2H), 1.97 (s, 3H), 1.9-2.0 (m, 4H), 1.25 (t, 3H), 1.07 (s, 6H) ppm.

### Example 9

### Synthesis of Oxime I-31

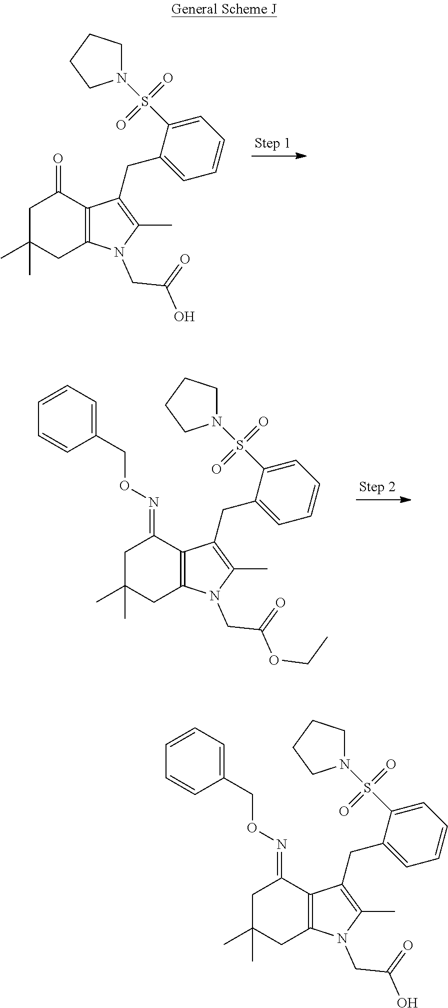 [SMILES: Cc1c(Cc2ccccc2S(=O)(=O)N2CCCC2)c2c(n1CC(=O)O)CC(C)(C)CC2=O ; CCOC(=O)Cn1c(C)c(Cc2ccccc2S(=O)(=O)N2CCCC2)c2c1CC(C)(C)C/C2=N\OCc1ccccc1 ; Cc1c(Cc2ccccc2S(=O)(=O)N2CCCC2)c2c(n1CC(=O)O)CC(C)(C)C/C2=N\OCc1ccccc1]

### Steps 1 and 2

### (E)-2-(4-(Benzyloxylmino)-2,6,6-trimethyl-3-(2-(pyrrolidin-1-ylsulfonyl)benzyl)-4,5,6,7-tetrahydro-III-indol-1-yl)acetic acid (I-31)

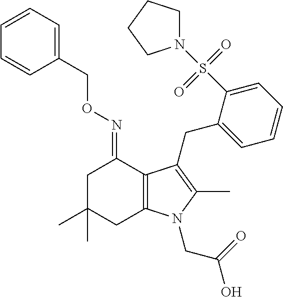 [SMILES: Cc1c(Cc2ccccc2S(=O)(=O)N2CCCC2)c2c(n1CC(=O)O)CC(C)(C)C/C2=N\OCc1ccccc1]

A mixture of 2-(2,6,6-trimethyl-4-oxo-3-(2-(pyrrolidin-1-ylsulfonyl)benzyl)-4,5,6,7-tetrahydro-1H-indol-1-yl)acetic acid (200 mg, 0.43 mmol) and O-benzylhydroxylamine hydrochloride (54 mg, 0.44 mmol) in absolute ethanol (2 mL) was prepared at room temperature. One drop of concentrated hydrochloric acid(aq) was added and the stirred mixture was heated overnight at 60° C. The solution was cooled to room temperature and diluted with ethyl acetate (60 mL), then washed with 2×10 mL water, dried over Na₂SO₄, filtered and concentrated by rotary evaporation. The major isomer was collected as a colorless glass (140 mg) by chromatography over SiO₂, eluting with hexane/ethyl acetate. The product was used directly in the next reaction without characterization. The colorless glass (140 mg, 0.21 mmol) was mixed in tetrahydrofuran/water (4 mL, 1:1) at room temperature. A solution of 1N NaOH(aq) (0.5 mL, 0.5 mmol) was added and the mixture was stirred 1 hr at room temperature. The mixture was diluted with 0.36 M NaH₂PO₄ (aq) (10 mL) and ethyl acetate (50 mL), the layers were well mixed and separated. The organic phase was washed with water (2×10 mL) and brine, then dried over Na₂SO₄, filtered and concentrated by rotary evaporation. (E)-2-(4-(Benzyloxyimino)-2,6,6-trimethyl-3-(2-(pyrrolidin-1-ylsulfonyl)benzyl)-4,5,6,7-tetrahydro-1H-indol-1-yl)acetic acid, (125 mg) was obtained as a white foam after chromatography over SiO₂ with hexane/ethyl acetate as eluent. ¹H-NMR (CDCl₃): δ (ppm): 7.94 (dd, 1H), 7.27 (dt, 1H), 7.21 (t, 1H), 7.02 (d, 1H), 7.05-7.25 (m, 5H), 4.81 (s, 2H), 4.53 (s, 4H), 3.3-3.4 (m, 4H), 2.53 (s, 2H), 2.40 (s, 2H), 1.96 (s, 3H), 1.8-1.9 (m, 4H), 1.05 (s, 6H) ppm.

### Example 10

### Biological Activity Measurements

### Animal Models Related to Allergic Response

Any of a variety of animal models and in vitro assays can be used to test the compounds for their effectiveness in reducing allergic and inflammatory activity. Useful compounds can exhibit effectiveness in reducing allergic response and inflammation in one or more animal models or in vitro assays.

### Induction of Contact Hypersensitivity.

In this model, induction of contact hypersensitivity (CHS) is created as described by Takeshita et al. (2004. Int. Immunol. 16(7):947-59). On days 0 and 1, female Balb/c mice, 7-8 weeks of age are painted onto the shaved abdominal skin with 400 μl of 0.5% fluorescein isothiocyanate (FITC) dissolved in acetone:dibutylpthalate (1:1, DBP). Six days later, mice are challenged by application of 20 μl of 0.5% FITC in DBP onto both sides of the right ear. The solvent control (DBP) is applied to the left ear. Challenge-induced increases in ear thickness are measured by an engineer's micrometer at 0, 24, 48 and 72 hours post-challenge. The CHS response is determined by challenge-induced increases in ear thickness. CHS response=[(right ear thickness post challenge−left ear thickness post challenge)−(right ear thickness pre challenge−left ear thickness pre challenge)].

To determine the presence of leukocyte infiltration, ears and back skins are fixed for 30 hours in zinc fixative at room temperature and embedded in paraffin for histological and immunohistochemical evaluation. For assessment of eosinophil peroxidase activity (EPO), skin sections are homogenized in 1 ml of ice cold buffer (0.05 M Tris-HCl pH 8.0 containing 0.1% Triton X-100). The tissue samples are centrifuged at 10,000 g for 20 minutes at 4° C. and supernatants are collected for measurement of EPO activity. In a 96 well microtiter plate, the substrate solution (100 μl of 10 mM o-phenylyenediamine in 0.05 M Tris-HCl and 4 mM H₂O₂) is added to the 20-fold diluted homogenate in buffer (100 μl). The reaction mixture is incubated at room temperature for 1 hour before the reaction is stopped by the addition of 100 μl of 2M sulfuric acid. The microtiter plate is measured for absorbance.

### Evan's Blue Test.

Complete protocol details can be found in Takeshita et al. (2004. Int. Immunol. 16(7):947-59). Briefly, female Balb/c mice, 7 weeks of age are injected at two locations intradermally on their shaved backs with increasing concentrations of 0.1-10 μg/site of DK-PGD₂. This is followed by an intravenous injection of 0.25 ml of saline containing 1.25 mg of Evan's blue dye. Four hours post-dye injection, mice are euthanized and the back skin is collected. Edema severity is assessed by measuring the density of the extravasated dye. Effects of pharmacological inhibition of the inflammatory reaction to DK-PGD₂ will also be assessed by treatment with CRTH2 antagonists, such as Ramatroban.

### Ovalabumin-Induced Airway Cell Proliferation and Inflammation.

Complete protocol details can be found in Eynon et al. (2003. J. Pharmacal. Ther. 304:22-29). Briefly, Brown Norway rats are sensitized on days 1, 2, and 3 with intraperitoneal (i.p.) injections of 1 mg ovalbumin (OVA) and 100 mg Al(OH)₃ in 1 mL 0.9% NaCl saline. They are then exposed to either 0.9% NaCl saline or 1% OVA aerosol every 3rd day (days 6, 9, & 12) for 30 minutes. 2 mg/kg dexamethasone is used as a positive control and is dosed i.p. once a day on days 4, 5, 6, 9, & 12. Vehicle (15% β-cyclodextrins in DMSO) and test compounds are dosed orally twice a day on days 5-12. On challenge days, all animals are treated 1 hour prior to OVA allergen exposure and, if required for twice a day treatment, ˜4-8 hours after allergen exposure. Samples are collected 24 hours after the last OVA challenge. For sample collection, rats are anaesthetized by administration of 10 mg/kg xylazine and 60 mg/kg ketamine intraperitoneally. Once the rats were fully anesthetized, blood is collected for serum via the retro-orbital route. The rats are subsequently perfused by injecting 30 mL PBS through the right ventricle of the heart after the abdominal aorta is severed. A tracheostomy is then performed and bronchoalveolar lavage fluid (BAL) is collected through five 5 mL rinses using Hank's Balanced Salt Solution, which was kept on ice. Airway inflammatory cell accumulation and proliferation of cells are measured through the BAL fluid collection and subsequent cell counts. Cytospin slides are prepared and eosinophil % are determined by counting ˜400 cells per slide. The test compounds are dosed at 5 mg/kg twice daily at various concentrations. Activity is scored based on the ability of the test compound to prevent ovalbumin-induced eosinophil induction (as determined by percentage of eosinophils in BAL fluid).

### Ovalbumin-Induced Airway Inflammation in Sensitized Brown Norway Rats

The assay assesses the effect of test compounds on cellular recruitment into the lung, after antigen challenge in the sensitised Brown Norway rat. The model is a slightly modified protocol based on that disclosed in Underwood et al. 2002 British Journal of Pharmacology 137: 263-0.275. Briefly, male Brown Norway rats (200-225 g, from Harlan) are be sensitised on days 0, 14 and 21 with ovalbumin (100 μg/rat, i.p.) administered with Alum™ (20 mg/rat aluminium hydroxide and 20 mg/rat magnesium hydroxide, i.p.). Rats are challenged with inhaled ovalbumin (10 g/l, 30 minutes) or saline aerosol on day 28. Vehicle (5 ml/kg) or test compound (1 or 10 mg/kg, 5 ml/kg) are dosed orally 16 and 1 hour(s) before and 1 and 6 hours after antigen challenge. Budesonide (3 mg/kg) is included as a positive control and dosed at the same time points. End point measurements are as follows; one hour after the challenge the rats have Enhanced Pause (PenH) levels monitored for 5 hours to assess late asthmatic reaction.

Cellular burden and inflammatory status are assessed. Twenty-four hours after ovalbumin challenge, rats are euthanised with an overdose of pentobarbitone i.p. A heparinised blood sample is taken via cardiac puncture and the resulting plasma kept frozen. Bronchoalveolar lavage (BAL) is carried out (2×3 ml RPMI media, 30 seconds each). Immediately after BAL, the left lobe is removed, perfused with RPMI to remove the blood pool of cells and 300 mg of lung is chopped and stored in RPMI/FCS (fetal calf serum) containing penicillin/streptomycin. The remaining perfused, chopped lung tissue is flash frozen and stored at −80° C. The remaining lung lobes are insufflated with formalin to a pressure of 20 mmHg, the lungs tied off and stored in formalin until required.

The 300 mg of tissue undergoes collagenase digestion and the cells are recovered (For method see Underwood et al., (1997) Br. J. Pharm., 122, 439-446). Total cell counts recovered from the airway lumen and lung tissue are quantified using a Sysmex cell counter. Differential cell counts (200 cells counted which comprise eosinophils, neutrophils, lymphomononuclear cells expressed as percentage and absolute cell counts) of cells recovered from the airway lumen and lung tissue are made by light microscopy from cytocentrifuge preparations stained with Wright-Giemsa stain. Remaining BAL samples are spun down and supernatant retained at 20° C. Additionally, this model can be used to assess the effect of agents described herein on airway resistance.

### Sephadex Induced-Pulmonary Eosinophilia in Rodents.

Male Swiss Webster mice are used in a model of Sephadex induced-Pulmonary Eosinophilia. In brief, test groups receive vehicle, test compound (10 mg/kg) or positive control, dexamethasone (0.5 mg/kg), by oral gavage, twice per day (p.o., b.i.d.) at a dosing volume of 10 ml/kg, on days −1, 0, 1 and once, 4 hours pre-sacrifice, on day 2. On day 0, test groups are each intravenously administered 3 mg/kg Sephadex beads G-100-120 (Sigma) at a dosing volume of 5 ml/kg or no Sephadex. On day 2, four hours post vehicle/test compound/dexmethasone administration, animals are euthanized by inhalation of CO₂ and subsequently undergo histopathologic and lavage evaluation of lungs for severity of eosinophilic infiltrate in peribronchiolar locations. Bronchoalveolar lavage fluid is collected by flushing the lung via the trachea 3 times with 1 ml aliquots of cold saline, and then the lungs are harvested by filling with formalin and allowed fixation a minimum of 1 day. White blood cell counts are prepared from lavage fluids. In addition, lavage fluids are immediately prepared for cytospin and cell differential counts performed. Cytospin slides are stained with a Wrights-Giemsa stain. Whole lung sections are stained with Hematoxylin and eosin stain for morphometry evaluation of severity of inflammatory cell infiltrate in peribronchiolar locations around Sephadex beads. Three sections (initial and 2 steps at 100 μm intervals) are prepared from each animal for analysis of area or diameter of inflammation around 5-8 Sephadex beads/mouse. Morphometric digital imaging analysis is performed to score inflammation. A similar experimental protocol can be performed using Lewis rats with the modification that animals are euthanized on day 1.

### Mouse Model of Allergic Airways Disease Using the FlexiVent System.

In this model, animals in groups of 10 (8-10 wk old male BALB/c mice) are used to assess allergic airway disease. Mice are quarantined for 14 days. On days 0 (the first day following the end of the 14 day quarantine) and day 7, experimental animals are immunized by intraperitoneal (i.p.) injection with a mixture of ovalbumin (OVA; 10 □g) and aluminum hydroxide (Alum; 2 mg) in sterile water. A second group of animals is immunized with sterile water only and serves as a nonimmunized (negative) control. On days 13, 14, 15, and 16, dexamethasone (positive control), test compound or vehicle only is delivered by oral gavage (all at 10 mg/kg and a dosing volume of 10 ml/kg) twice a day. Animals are exposed to ovalbumin on days 14 and 15. Ovalbumin exposures are generated by aerosolizing 1% heat-aggregated ovalbumin (chicken egg, grade V; Sigma, St. Louis, Mo.), diluted with filtered air, and then delivered to the exposure chambers for 3 hours (H2000, Hazelton Systems). The total mass concentration of ovalbumin is determined by gravimetric analysis of filter samples taken every hour during exposure. The target mass concentration of ovalbumin is 4 mg/m³. Chamber temperatures are maintained at 26±2° C. and lights on a 12 hour on/off cycle. Animals are given food (Teklad™ certified rodent diet (Harlan Teklad, Madison, Wis.)), ad libitum except during the 3 hour exposure period. Water is available ad libitum throughout the duration of the study.

On day 17, animals are anesthetized and tested for pulmonary function (response to methacholine challenge) by forced oscillation techniques (FlexiVent). Airway hyperresponsiveness (AHR) to increasing concentrations of aerosolized methacholine (MCh) is measured using a FlexiVent analyzer (SCIREQ, Montreal, Canada). Briefly, each mouse is anesthetized with Avertin (250 mg/kg; 0.02 ml/g; 1.2% (w/v) solution of 2,2,2 tribromoethanol in 0.8% tert-amyl ethanol (2 methyl, 2 butanol)) i.p. and placed on a heating pad. The neck fur is shaved and a small superficial incision made in the skin above the trachea. After the lobes of the salivary gland are separated, a small incision is made in the trachea, and the trachea is cannulated with a blunt-end 20 gauge needle hub. The cannula is secured by suture thread and the skin is pulled back and secured by cyanoacrylate adhesive. Ventilation is performed through the cannula by positive pressure maneuvers on the Flexivent apparatus. Once on the ventilator, pancuronium, (paralytic, 0.5 mg/kg) is administered i.p. Heart rate is monitored via a Grass Instruments Recorder w/Tachograph. Changes in heart rate greater than 50 bpm from baseline require supplementing the anesthesia (Avertin, ip). Additional doses of Avertin are given at a dose of 100 mg/kg and the animal's heart rate is monitored for at least 60 sec to determine if additional doses are needed. After baseline measurements of resistance and compliance, increasing doses of methacholine (Mch; 3, 6, 12, 25, 50 mg/ml nebulizer) are delivered via aerosol and resistance and compliance are measured. Airway resistance is calculated for each concentration of methacholine and the average+SEM is plotted for all treatment groups. Changes in pulmonary resistance (i.e., Mch dose-response curves) are assessed by repeated measures two way analysis of variance (ANOVA) with Bonferroni post-test. All other statistical comparisons are made using ANOVA with the Dunnetts multiple comparison test. A value of p<0.05 is considered significant.

Following AHR measurements, blood is collected and saved for further evaluation. The animals are then euthanized by injection with a lethal dose of a pentobarbital-based euthanasia solution. Bronchoalveolar lavage (BAL) cells are obtained from 7 animals per experimental or control group by inserting a catheter into the trachea and lavaging the lung 3 times with 0.8 ml of PBS (without calcium chloride and magnesium chloride). Total BAL cells are determined using a hemacytometer. BAL cells are spun onto slides by cytocentrifugation and stained with a modified Wright-Giemsa stain. Four hundred cells are counted and the percentage of specific cell types determined for each animal. The first lavage fluid sample (after centrifugation) is frozen separately for future cytokine analysis. The whole lung is snap frozen dry for future analyses.

Three animals from each group which are not subjected to BAL are used for histopathologic analysis and have their lungs instilled via the trachea with 10% buffered formalin, removed and fixed in the same solution. Generally, three specimens per treatment, each consisting of multiple axial sections of lung, are examined. All sections are stained with alcian blue-H&E. Lesions are graded on a subjective basis. Lesions are graded as minimal, mild, moderate, and marked (corresponding to severity scores of 1, 2, 3, and 4, respectively) and given a distribution designation of either focal, locally extensive, multifocal, multifocal and coalescing, or diffuse (corresponding to distribution scores of 1, 2, 3, 4 and 5, respectively). The product of the severity and distribution scores is averaged for each treatment group.

### Prostaglandin D₂-Induced Eosinophilic Airway Inflammation.

Complete protocol details can be found in Shiraishi et al (2004. J. Pharmacol. Ther. epub as DOI:10:1124/jpet.104.078212). Briefly, Brown Norway rats are intravenously injected with rat interleukin-5 or PBS, one hour prior to intratracheal administration of prostanoid receptor agonists. These agonists can include the following; PGD₂, two CRTH₂-specific agonists, DK-PGD₂, 15R-methyl PGD₂, and 11-deoxy-11-methylene-15-keto-PGD₂ (MK-PGD₂), a DP receptor-specific agonist BW 245C, a thromboxane A₂ receptor (TP)-specific agonist, -BOP and Indomethacin. In some experiments, an orally delivered CRTH2/TP antagonist, Ramatroban, an intravenously delivered DP antagonist, BW A868C, or an intravenously delivered TP antagonist are administered two hours prior to administration of agonists. Rats are euthanized at 2, 8 and 24 hours post-agonist administration. Inflammatory cell accumulation in the trachea and lungs is recovered by bronchoalveolar lavage for cell counts and lungs are evaluated by histological examination. In a separate experiment, rats receive intravenous injection of IL-5 (0.2 ng/kg) or PBS one hour prior to intratracheal administration of PGD₂ (100 nmoles/animal) or vehicle. A peripheral blood sample is collected hourly post-dose of IL-5 for hematological evaluation.

### Murine Allergic Inflammation.

Complete protocol details are described in Fujitani et al. (2002 J. Immunol. 168:443-449) and Matsuoka et al. (2000. Science 287: 2013-2017). Briefly, transgenic and wildtype mice are immunized with 10 μg ovalbumin (OVA) in 0.2 ml aluminum hydroxide (Alum) on days 0 and 14. On day 21, the mice are exposed to aerosolized OVA (50 mg/ml in sterile saline) for 20 minutes. On days 1 and 3 post-OVA challenge, mice are euthanized, bronchoalveolar lavaged, and the lavage fluid is assessed by differential cell counting.

### Allergic Rhinitis in Anesthetized Rodents.

In this model described, for example, by Arimura et al. (2001 J. Pharmacol. Ther. 298:411-419) guinea pigs are sensitized to OVA twice by inhalation of an aerosol solution of 1% OVA for 10 minutes. At 7 days after the second sensitization, the animals are anesthetized and artificially ventilated through a tracheal cannula using a respirator. Another glass cannula is inserted into the nasopharynx from the side of the larynx, and a fixed amount of air is continuously insufflated into the nasal cavity via the nasal cannula using another respirator. Insufflation pressure is monitored by a pressure transducer connected to the side arm of the nasal cannula as an indication of intranasal pressure. Nasal antigen challenge is performed by generating an aerosol of 3% OVA between the nasal cannula and the animal respirator for 3 minutes using an ultrasonic nebulizer, and then the intranasal pressure is measured for 30 minutes. Nasal secretion and the nose are collected for further evaluation.

A biphasic allergic rhinitis model in conscious guinea pigs is also fully described in Arimura et al. (2001 J. Pharmacol. Ther. 298:411-419).

### Allergic Conjunctivitis Model.

Complete protocol details are described in Arimura et al. (2001 J. Pharmacol. Ther. 298:411-419). Briefly, a 2.5% OVA solution is applied topically to both eyes (10 μl/eye) of conscious guinea pigs that have been sensitized as described in the “Allergic Rhinitis Model in Anesthetized Rodents” protocol above. Immediately following OVA application, Evan's blue dye (20 mg/kg i.v.) is injected as a marker of plasma exudation. The amount of Evan's blue extravasated in the conjunctiva and eyelid for 30 minutes is quantified. Independently, histamine 0.001%, PGD2 0.01%, or a combination of the two are applied to the eyes of nonsensitized guinea pigs, and dye exudation is determined.

### Determination of Interleukin-13 Levels in Bronchial Alveolar Lavage Fluid.

A commercially available ELISA kit'(Biosource, Catalog #KRC0132) is used to determine the effects of compounds on the Interleukin-13 (IL-13) levels of bronchial alveolar lavage fluid (BALF) taken from rats that have undergone certain allergen induced (e.g. ovalbumin, sephadex, prostaglandin D₂) airway cell proliferation and inflammation.

After collection, BALF samples are concentrated 5-fold with Microcon YM-3 centrifugal devices (Millipore, Catalog #42404) and stored at −80° C. until use. A 500 pg/mL standard stock is prepared by reconstituting the IL-13 standard provided in the kit with the amount of standard diluent specified on the standard vial. A standard curve is then prepared by serially the standard stock down to 7.8 pg/mL. 50 □L of each point of the standard curve and 50 μL of concentrated BALF sample are added to the ELISA plate. Added to these samples is 150 μL of anti-rat IL-13 biotin conjugate. The plate is then incubated at room temperature for 2 hours. The plate is then washed 4 times with wash buffer and 100 μL of 1-x streptavidin-peroxidase is added to all wells. The samples are then incubated at room temperature for 30 minutes. Again, the plate is washed 4 times with wash buffer. 100 μl of stabilized chromogen are added to each well and the plate is incubated at room temperature for 45 minutes. To stop the reaction, 100 μL of stop solution is added and the plate is read at 450 nm. Levels of other cytokines including IL-1β, IL-4, IL-5 and the chemokine, eotaxin can be similarly assessed in BALF samples to determine the effect of test compounds on Th-2 related function.

### Determination of Ovalbumin Specific Immunoglobulin E in Serum.

The effects of compounds on serum immunoglobulin E (IgE) levels in rodents that have undergone allergen-induced (e.g. ovalbumin) airway cell proliferation and inflammation can be measured using an assay developed with reference to Salgado et al., Allergol. et Immunopathol., 16, 2 (95-98), 1988. Serum samples are taken from rats suffering from asthma, induced by the inhalation of ovalbumin, and stored at −80° C. until use. The ELISA plate is coated with 1.25 mg/mL ovalbumin prepared in coating buffer (0.5M Carbonate-Bicarbonate, pH 9.6, Bethyl Labs, Catalog #E107) and incubated overnight at 4° C. After 18 hours, the plate is washed one time with wash buffer (50 mM Tris, 0.14 M NaCl, 0.05% Tween 20, pH 8.0, Bethyl Labs, Catalog #E106). 200 tit of blocking solution (5% skim milk/PBS) is added and the plate is incubated at 4° C. for 1 hour. Serum samples are diluted 1:3000 in sample diluent (Post coat solution containing 50 mM Tris, 1% BSA, pH 8.0 0.05% Tween 20, Bethyl Labs, Catalog #E104). After the one hour incubation with blocking solution, the plate is washed three times with wash solution and 100 μL of diluted sample is added to the appropriate well. Samples are then incubated at room temperature for 3 hours. Once the 3 hour incubation is complete, the plate is washed five times with wash buffer. The sheep anti-rat IgE HRP conjugate detection antibody (Bethyl Labs, Catalog #A110-117P) is diluted 1:100 in a 1% skim milk/PBS solution. 100 in of this solution is then added to the plate and the plate is incubated for 1 hour at 4° C. The plate is then washed another five times with wash buffer. The TMB peroxidase substrate (Bethyl Labs, Catalog #E102) is prepared by adding equal volumes of TMB peroxidase substrate with Peroxidase solution B. 100 μL of substrate is added to plate and incubated at room temperature for 15 minutes. The enzymatic reaction is stopped by adding 100 μL of 2 M sulfuric acid (Sigma Aldrich). The plate is then read at a wavelength of 450 nm.

### Determination of Methacholine Responsiveness in Mice 2-8 Weeks of Age

Complete protocol details for this model can be found in Bozanich et al. (J Appl Physiol 103: 542-546, 2007). Briefly, the animals are prepared, anaesthetized, tracheotimized, connected to a ventilator, and cannulated as described. Two small electrodes are placed into the intercostal muscles of the mouse and connected to an electrical stimulator (Grass Instruments, Quincy, Mass.). Ventilation is paused, the positive end-expiratory pressure is removed with the airway, and the plethysmograph is opened to atmosphere to allow the lungs to reach the elastic equilibrium volume at transrespiratory pressure of 0 hPa, defined as functional residual capacity (FRC). With the plethysmograph closed and the airway occluded, five to eight stimulated breathing efforts are induced over a 10 sec period. FRC is then calculated using Boyle's principle. Lung volume (VL) is then increased by lowering the plethysmograph pressure from 0 to −20 hPa in a quasi-linear fashion during 15-20 sec. The increase in VL from FRC to transrespiratory pressure=20 hPa (VL20) achieved during the slow deep inflation (sDI) maneuver is determined by integrating the flow into the animal through the wave tube as described. The inflation phase is followed by a slow passive expiration to transrespiratory pressure=0 hPa, where the measurement of FRC is repeated in a subgroup of animals. Respiratory system impedance (Zrs) is measured using a low-frequency (4-38 Hz) forced oscillation technique and a wave-tube system as described. Doubling doses (6-48 ug/min-kg) of beta-methacholine chloride (MCh; Sigma-Aldrich) are delivered for 5 min by constant infusion via the jugular vein cannula. A steady-state constriction is achieved by 5 min and is verified by monitoring tracheal pressure during mechanical ventilation. FRC is measured, and a single slow deep inhalation (sDI) maneuver is performed with the infusion continuing to run. Test compound or vehicle alone is administered, for example, orally, twice daily for 1-4 days prior to receiving the MCh treatment and may also include dosing approximately 4 hours before MCh treatment. Differences in FRC before and after an sDI maneuver performed at baseline and the maximum MCh dose in a mice subgroups (for example, 3-10 mice from each age group) are determined using paired t-tests. MCh responsiveness in the presence and absence of test compound is calculated as described for each animal group.

### Murine Model of Atopic Dermatitis.

This model is described, for example, by Spergel et. al. (1998 J. Clin. Invest. 101: 1614-1622). Epicutaneous (EC) sensitization of mice was performed as described by Wang et al. (1996 J. Immunol. 156:4079-4082). Briefly, 4-6 week old BALB/c mice were anesthetized with methoxyflurane (Metofane; Mallinckrodt Veterinary, Mundelein, Ill.), then shaved with an electric razor. 100 μg of OVA (grade V; Sigma Chemical Co., St. Louis, Mo.) in 100 μl of normal saline or placebo (100 μl of normal saline) was placed on a 1×1 cm patch of sterile gauze, which was secured to the skin with a transparent bioocclusive dressing (Johnson and Johnson Medical Inc., Arlington, Tex.). The patch was placed for a 1-wk period and then removed. 2 wk later, an identical patch was reapplied to the same skin site. Each mouse had a total of three 1-wk exposures to the patch separated from each other by 2-wk intervals. Inspection confirmed that the patch remained in place at the end of each sensitization period. For a positive control, intraperitoneal (IP) sensitization of another group of mice was performed with OVA (100 μg)-alum and boosted 2 wk later with the same dose of OVA in alum.

Mice are bled and sera collected 1 hour following the end of the series of three EC sensitizations by the standard PharMingen ELISA protocol used to quantify the total amount of IgE in serum. OVA specific antibodies in the serum can also be assessed, as well as cellular infiltrate into the skin by histological and immunohistochemical analysis. Also, the presence of mRNA for cytokines in skin sites sensitized with OVA can be detected via RT-PCR (protocol details are fully described in Spergel et. al., 1998 J. Clin. Invest. 101: 1614-162).

BAL fluid can also be examined in this model. EC sensitized mice are challenged with a single exposure to inhaled 1% OVA via a nebulizer for 20 minutes, and 24 hours later BAL fluid is examined for the presence of eosinophils and other cellular infiltrate (protocol details are fully described in Spergel et. al., 1998 J. Clin. Invest. 101: 1614-162).

Airway hyperresponsiveness can also be assessed in this model described by Spergel et. al., 1996. Briefly, 24 hours after one dose of nebulized 1% OVA, airway measurements are measured plethysmographically in sedated, ventilated mice in response to graded doses of intravenous methacholine.

### DK-PGD2-Induced Systemic Eosinophilia in Rats.

Female Sprague-Dawley rats (175-250 g) were dosed orally with test compound (or vehicle). Thirty minutes after dosing, animals were anaesthetized with isoflurane. Following induction of anaesthesia, animals received an intracardiac injection of 10 μg DK-PGD2 in 0.3 ml heparinized (10 U/ml) saline. Control animals received an injection of 0.3 ml heparinized saline. Sixty minutes after the intracardiac injection, animals were again anesthetized with isoflurane and a blood sample was drawn from the abdominal aorta (into heparin) while the rat was anaesthetized but not dead. An aliquot of blood (5004) was mixed with an equal volume of 4% dextran (mw 500,000) and the erythrocytes were allowed to settle. A cytospin preparation was made from the resulting leukocyte rich fraction (top) and the cytospin was fixed and stained with Diff-Quick Stain kit (Dade Behring Inc, Newark, Del.). An aliquot of the leukocyte rich fraction was taken for total leukocyte count using flow cytometer (Guava EasyCyte Mini system). Differential leukocyte counts were obtained from the cytospin preparations. Blood eosinophil numbers were determined from the total leukocyte count and the percentage eosinophils.

Human Whole Blood CD111b Antagonist Assay (Modified from Nicholson, et al. Pulmonary Pharmacology and Therapeutics: 20 (2007); 52-59).

The potential CRTH2 antagonist activity of certain compounds was tested in human whole blood using an assay that tests the ability of the compounds to block the CD11b expression in eosinophils by 15-R-methyl-PGD2. A CRTH2 antagonist should block CD11b expression by subsequently added 15-Methyl-PGD2. Human whole blood (200 μL) was incubated at 37° C. for 10 minutes in the presence of various concentrations of test compounds before being challenged with the agonist 1-5R-Methyl-PGD2 (10 nM). Reactions were terminated by the addition of ice-cold PBS+0.5% BSA+2 mM EDTA (1 mL) and centrifugation (300×g for 5 minutes at 4° C.) Cells were then incubated at 4° C. for 10 min in the presence of human IgG. Cells were then incubated for 30-45 min with a mixture of PE-Cy5-labeled mouse anti-human CD16 (10 ul; BD Biosciences) and FITC-labeled mouse anti-human CD11b (10 μL; Beckman Coulter.) After rinsing (1 mL ice-cold PBS+0.5% BSA+2 mM EDTA), red blood cells were lysed by the addition of 1 mL ice-cold H₂O to the cell pellet for 30 sec-1 min immediately followed by the addition of 3.5% NaCl (300 μL.) Cells were then rinsed (2×−1 ml ice cold PBS+0.5% BSA+2m MEDTA) and fixed in PBS containing 1% formaldehyde. The distribution of fluorescence intensities was measured by flow cytometry. Eosinophils were gated out on the basis of their granularity (high side scatter) and absence of CD-16. CD11b was then measured on this eosinophil population on the basis of fluorescence due to FITC.

### DPBS CD111b Antagonist Assay.

The potential CRTH2 antagonist, activity of certain compounds was tested using a CD11b expression assay using essentially the method described by Monneret et al. (J Pharmacol Exp Ther 304:349-55, 2003). Briefly, polymorphonuclear cells (0.5 ml; 10⁶/ml cells) in PBS containing 0.9 mM CaCl₂ and 0.5 mM MgCl₂) were preincubated with various concentrations of test compounds at room temperature for 10 minutes before they were challenged with the agonist 15R-Methyl-PGD2 (10 nM). The incubations were terminated by the addition of ice-cold FACSFlow (BD Biosciences; Cat#342003) and centrifugation (400 g for 5 minutes at 4° C.). The cells were then incubated for 30 minutes at 4° C. with a mixture of PE-labeled mouse anti-human VLA-4 (5 μl; BD Biosciences) and FITC-labeled mouse anti-human CD11b (10 μl; Beckman Coulter). The cells were then incubated with Optilyse C (0.25 ml; Beckman Coulter) for 15 minutes, centrifuged, and then fixed in PBS (0.4 ml; calcium and magnesium free) containing 1% formaldehyde. The distribution of fluorescence intensities among 60,000 cells was measured by flow cytometry. Eosinophils were gated out on the basis of their granularity (high side scatter) and labeling with VLA-4 (PE fluorescence). CD11b was then measured in the eosinophil region on the basis of fluorescence due to FITC. All data were corrected for the value obtained for the corresponding isotope control antibody.

Data for compounds of the invention are summarized in Table 2.

TABLE 2

| | CD11b | CD11b |
|---|---|---|
| | Antagonist | Antagonist |
| | DPBS | Human Whole |
| No. | IC₅₀(nM)* | Blood IC₅₀(nM)* |
| I-1 | A | Not determined |
| I-2 | B | E |
| I-3 | A | C |
| I-4 | E | E |
| I-5 | A | C |
| I-6 | C | F |
| I-7 | A | D |
| I-8 | D | F |
| I-9 | E | F |
| I-10 | B | E |
| I-11 | E | F |
| I-12 | E | F |
| I-13 | B | D |
| I-14 | E | F |
| I-15 | A | C |
| I-16 | D | F |
| I-17 | A | C |
| I-18 | E | F |
| I-19 | B | D |
| I-20 | A | C |
| I-21 | A | D |
| I-22 | A | D |
| I-23 | A | B |
| I-24 | A | C |
| I-25 | A | D |
| I-26 | E | G |
| I-27 | A | D |
| I-30 | B | E |
| I-31 | B | E |
| I-32 | F | F |
| I-33 | B | E |
| I-34 | C | F |
| I-35 | B | D |
| I-36 | B | D |
| I-37 | A | D |
| I-38 | B | F |
| I-39 | A | C |
| I-40 | B | E |
| I-42 | A | A |
| I-43 | C | D |
| I-44 | B | E |
| I-45 | E | G |
| I-46 | A | C |
| I-47 | C | D |
| I-48 | A | B |
| I-49 | B | D |
| I-50 | A | B |
| I-51 | E | G |
| I-52 | A | C |
| I-53 | Not | F |
| | Determined | |

*Data Codes:
A = 0 to <1
B = 1 to <5
C = 5 to <20
D = 20 to <100
E = 100 to <500
F = 500 to <3000
G = 3000 or >3000

A number of embodiments have been described. Nevertheless, it will be understood that various modifications may be made without departing from the spirit and scope of the invention.

1. A compound represented by Formula I, or a pharmaceutically acceptable salt thereof; 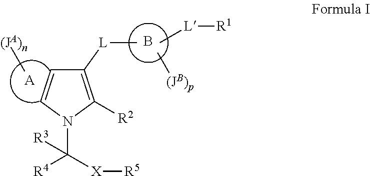
wherein:
Ring A is a 5 to 10-membered cycloaliphatic ring or a 4 to 10-membered heterocycle; wherein said heterocycle contains from 1 to 3 ring heteroatoms independently selected from N, O or S;
n is an integer selected from 0 to 6;
each occurrence of J^{A} is independently selected from halogen, C₁₋₄ alkyl, C₁₋₄ haloalkyl, C₁₋₄ alkoxy, C₁₋₄ haloalkoxy, —CN, —OH, oxo, ═NOR⁶, —NH(C₁₋₄ alkyl), —N(C₁₋₄ alkyl)₂ or —NH₂;
R⁶ is selected from C₁₋₆ alkyl or (C₁₋₄ alkyl)-(C₆₋₁₀ aryl);
Ring B is a monocyclic or bicyclic ring selected from phenyl or a 5 to 10-membered heteroaryl ring; wherein said heteroaryl ring contains 1 to 3 ring heteroatoms independently selected from N, O or S;
p is an integer selected from 0 to 2;
each occurrence of J^{B} is independently selected from halogen, —NO₂, —CN, —OH, —SH, —NH₂, C₁₋₄ alkyl, C₁₋₄ haloalkyl, —O(C₁₋₄ alkyl), —O(C₁₋₄ haloalkyl), —S(C₁₋₄ alkyl), —NH(C₁₋₄ alkyl) or —N(C₁₋₄ alkyl)₂;
L is a linker selected from a methylene, —O—, —S(O)ₘ— or —NR⁹—; wherein said methylene is optionally and independently substituted with 1 or 2 instances of halogen or C₁₋₄ alkyl; wherein m is an integer selected from 0 to 2;
R⁹ is selected from hydrogen or C₁₋₄ alkyl;
L′ is a linker selected from —Y—SO₂, —Z—C(O)—, —C(O)—Z— or a C₁₋₂ alkylene group; wherein:
when ring B is phenyl, Y is selected from a methylene or —NR¹⁰— and Z is selected from a single bond, a methylene or —NR¹⁰—, or
alternatively, when ring B is phenyl, Y is a single bond, L′ is a —SO₂— group between ring B and R¹ and R¹ is a 5 to 6-membered heterocyclic ring which is linked to the —SO₂— group through a ring N atom; wherein said 5 to 6 membered heterocyclic ring optionally contains an additional heteroatom selected from N, O or S and is optionally and independently substituted by up to two instances of R⁸; or
alternatively, when ring B is a 5 to 10-membered heteroaryl ring, Y is selected from a single bond, a methylene or —NR¹⁰— and Z is selected from a single bond, a methylene or —NR¹⁰—;
R¹⁰ is selected from hydrogen, C₁₋₆ alkyl or a —(C₁₋₄ alkyl)-(C₆₋₁₀ aryl) group;
R¹ is selected from C₁₋₆ aliphatic, C₁₋₆ haloaliphatic or a monocyclic ring selected from a 3 to 8-membered cycloaliphatic, a phenyl ring, a 5 to 6-membered heteroaryl or a 4 to 8-membered heterocycle; wherein said heteroaryl or heterocycle contains from 1 to 3 ring heteroatoms independently selected from N, O or S; and wherein when R¹ is a ring, it is optionally and independently substituted with up to three instances of R⁸;
each occurrence of R⁸ is independently selected from halogen, C₁₋₄ alkyl, C₁₋₄ haloalkyl, a 6 to 10 membered arylalkoxy group, C₁₋₄ alkoxy, —C(O)O(C₁₋₄ alkyl), —C(O)(C₁₋₄ alkyl), —CN, —OH, —NH₂, —NH(C₁₋₄ alkyl) or N(C₁₋₄ alkyl)₂;
R² is selected from hydrogen, halogen, C₁₋₄ alkyl, C₁₋₄ haloalkyl, C₁₋₄ alkoxy, C₁₋₄ haloalkoxy, CN or OH;
R³ and R⁴ are each independently selected from hydrogen, deuterium, halogen, C₁₋₆ alkyl or C₁₋₆ haloalkyl; or R³ and R⁴ taken together with the atom to which they are attached form a C₃₋₇ cycloalkyl ring;
X is selected from a direct single bond or a methylene linker, wherein said methylene is optionally and independently substituted with up to two instances of halogen, C₁₋₄ alkyl or C₁₋₄ haloalkyl;
R⁵ is selected from —C(O)OR⁷, —C(O)O—R¹¹, —C(O)N(R⁷)₂, —C(O)NOR⁷ or —C(O)NSR⁷;
each occurrence of R⁷ is independently selected from hydrogen or C₁₋₄ alkyl; and
each occurrence of R¹¹ is independently selected form a —(C₁₋₆ alkyl)-(C₆₋₁₀ heteroaryl), a —(C₁₋₆ alkyl)-(C₆₋₁₀ heterocyclyl) or a —(C₁₋₆ alkyl)-(C₆₋₁₀ aryl) wherein each of said heteroaryl and heterocyclyl contains 1 to 3 ring heteroatoms independently selected from N, O or S; and wherein R¹¹ is optionally and independently substituted with up to three instances of R⁸.

2. The compound of claim 1 wherein R³ and R⁴ are each independently selected from hydrogen or a methyl, wherein the methyl is optionally and independently substituted with 1 to 3 instances of halogen; or, alternatively, R³ and R⁴ taken together with the atom to which they are attached form a cyclopropyl ring.

3. The compound according to claim 2, wherein R³ and R⁴ are both hydrogen.

4. The compound according to claim 1, wherein Ring A is a 5 to 7-membered cycloaliphatic ring or a 6-membered heterocycle; wherein said heterocycle contains 1 ring heteroatom selected from N or O.

5. The compound according to claim 4, wherein ring A is a cyclohexane ring fused to the pyrrole ring and the compound is represented by Formula IV;

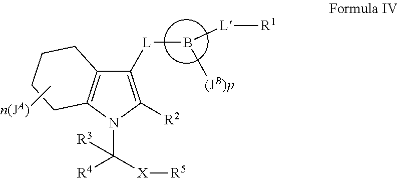

6. The compound of claim 5, wherein two instances of J^{A} are each a methyl attached to the same ring carbon.

7. The compound of claim 6 represented by Formula V or Formula VI: 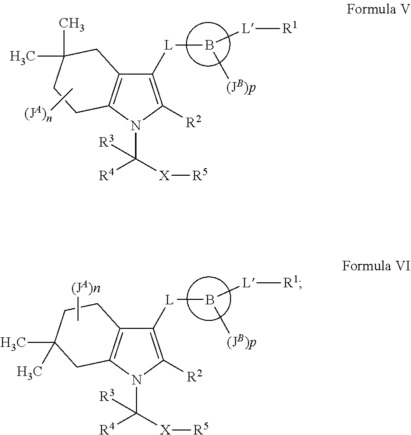
wherein n is an integer selected from 0 to 4.

8. The compound of claim 7, wherein n is zero.

9. The compound of claim 7, wherein n is 1, and J^{A} is oxo, ═NOCH₃, ═NOCH₂CH₃, ═NO(t-butyl) or ═NOCH₂C₆H₅.

10. The compound of claim 4, wherein ring A is a tetrahydro-2H-pyran ring fused to the pyrrole ring and the compound is represented by Formula VII;

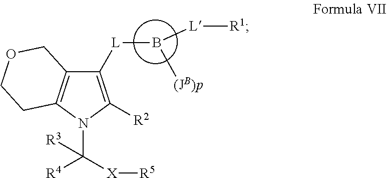

11. The compound of claim 4, wherein ring A is a piperidin-2-one ring fused to the pyrrole ring and the compound is represented by Formula VIII:

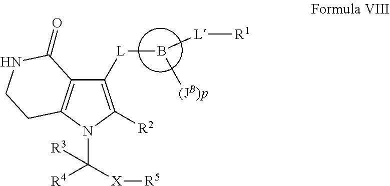

12. The compound of claim 1, wherein ring B is selected from phenyl, a monocyclic 5 to 6-membered heteroaryl ring or a bicyclic 9-membered heteroaryl ring; wherein said mono or bicyclic heteroaryl ring contains up to three ring heteroatoms independently selected from N, O or S.

13. The compound of claim 12, wherein ring B is phenyl, thiophenyl, a 6-membered heteroaryl or a 9-membered bicyclic heteroaryl ring; wherein said 6 or 9-membered heteroaryl ring contains 1 or 2 ring nitrogen atoms.

14. The compound of claim 13, wherein ring B is selected from phenyl, thiophenyl, pyridyl or indolyl.

15. The compound of claim 14, wherein ring B is phenyl and the compound is represented by Formula IX:

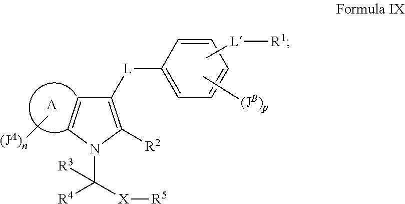

16. The compound of claim 14, wherein ring B is pyridyl and the compound is represented by Formula X:

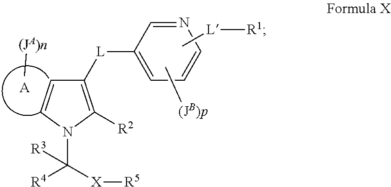

17. The compound of claim 14, wherein ring B is thiophenyl and the compound is represented by Formula XI:

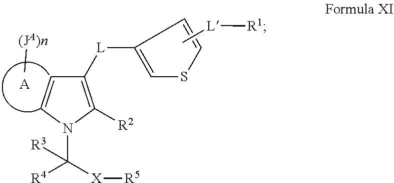

18. The compound of claim 14, wherein ring B is indolyl and the compound is represented by Formula XII:

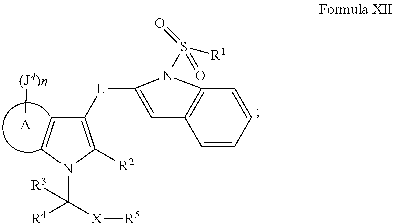

19. The compound of claim 1, wherein L is selected from —CH₂—, —O—, —S—, —SO— or —SO₂—.

20. The compound of claim 19, wherein L is selected from —CH₂— or —S—.

21. The compound of claim 15, wherein L′ is —NR¹⁰SO₂— and R¹⁰ is selected from hydrogen or C₁₋₄ alkyl.

22. The compound of claim 15, wherein L′ is —SO₂— and R¹ is selected from one of the rings depicted below; wherein L′ is attached either ortho or para to L on ring B.

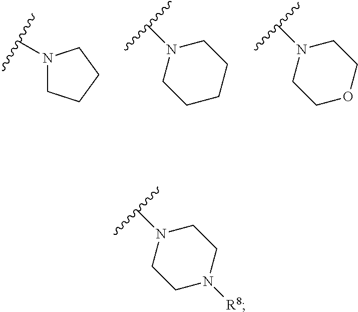

23. The compound of claim 15, wherein L′ is attached ortho or para to L on ring B.

24. The compound of claim 15, wherein R¹ is selected from a 3 to 6-membered cycloaliphatic ring, phenyl, a 6-membered heteroaryl or a 4 to 8-membered heterocycle; wherein said heteroaryl or heterocycle contains 1 or 2 heteroatoms selected from O and N; and wherein R¹ is substituted with up to three instances of R⁸.

25. The compound of claim 24, wherein R¹ is phenyl, a cyclopropyl ring or a 5 to 6-membered heterocyclic ring; wherein said phenyl or heterocyclic ring is optionally and independently substituted with up to 3 instances of R⁸.

26. The compound of claim 25, wherein R¹ is phenyl and the compound is represented by Formula XIII. 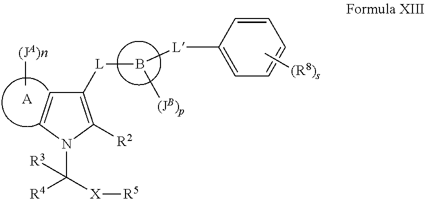
wherein s is an integer selected from 0 to 2.

27. The compound of claim 1, wherein R⁸ is chosen from C₁₋₄ alkyl, halogen, methoxy, —OH, —C(O)O(C₁₋₄ alkyl), —O(benzyl) or C₁₋₄ haloalkyl.

28. The compound of claim 1, wherein R² is selected from hydrogen, fluoro, methyl, ethyl or trifluoromethyl.

29. The compound of claim 28, wherein R² is methyl.

30. The compound of claim 1, wherein X is a single bond.

31. The compound of claim 1, wherein R⁵ is —C(O)OR⁷ and R⁷ is hydrogen or C₁₋₄ alkyl.

32. The compound of claim 31, wherein R⁵ is selected from C(O)OH, C(O)OCH₃ or C(O)OCH₂CH₃.

33. The compound of claim 32, wherein R⁵ is —C(O)OH

34. The compound of claim 1, wherein the compound is represented by Formula XIV: 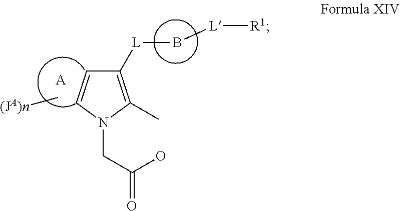
wherein:
ring A is selected from a cyclopentane ring, a cycloheptane ring, a cyclohexane ring, a tetrahydropyran ring or a piperidine ring, each of them fused to the pyrrole ring;
each J^{A} is independently selected from oxo or methyl;
n is 0, 2 or 3;
L is —CH₂— or —S—;
ring B is selected from a thiophenyl ring, a pyridyl ring or an indolyl ring and L′ is —SO₂—; wherein L′ is ortho to L on ring B; and R¹ is selected from:
(a) phenyl, optionally substituted by 0 to 2 instances of R⁸;
(b) cyclopropyl ring;
(c) methyl;
(d) a 5 to 6-membered heterocycle containing 1 or 2 heteroatoms selected from N or O;
or, alternatively,
ring B is phenyl and L′ is —SO₂— or —NR¹⁰SO₂—; wherein L′ is ortho to L on ring B; and R¹ is chosen from: N-pyrrolidinyl, N-morpholinyl, N-piperidinyl or N-piperazinyl; said N-piperazinyl optionally substituted by R⁸ on the second nitrogen atom;
or, alternatively,
Ring B is phenyl and L′ is NR¹⁰SO₂—; wherein L′ is ortho to L on ring B; and R¹ is selected from:
(a) phenyl, optionally substituted by 0 to 2 instances of R⁸;
(b) cyclopropyl ring;
and R¹⁰ is selected from hydrogen, ethyl or methyl.

35. The compound of claim 34, wherein the compound is represented by Formula XV or Formula XVI:

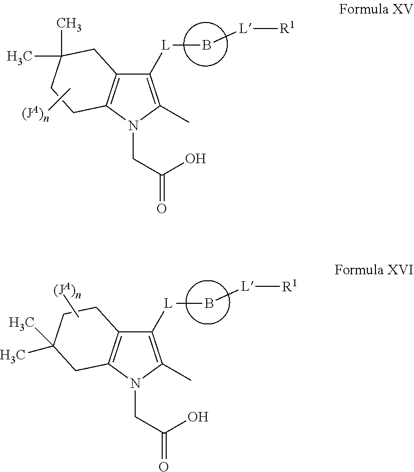

36. The compound of claim 34, wherein L′ is —SO₂— and ring B is a thiophenyl ring, a pyridyl ring or an indolyl ring.

37. The compound of claim 35, wherein n is 1, J^{A} is oxo and the compound is represented by Formula XVII.

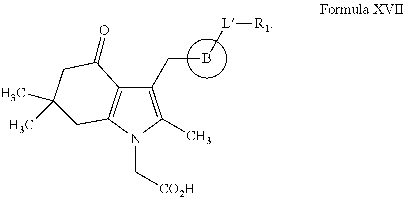

38. The compound of claim 37, wherein the compound is represented by Formula XVIII, XIX or XX:

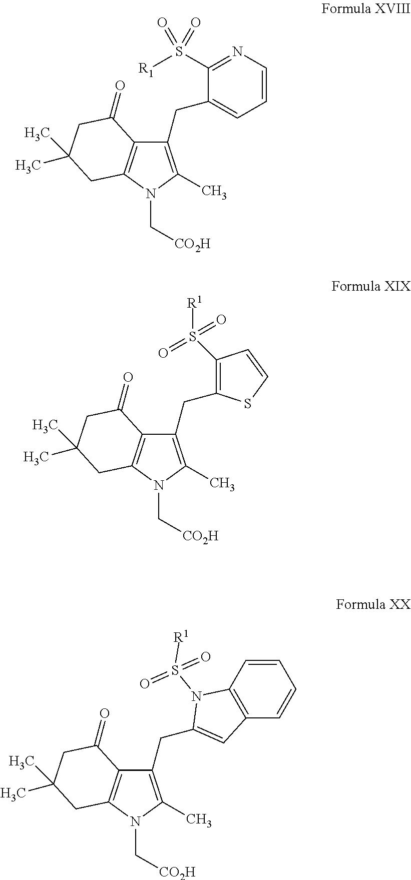

39. The compound of claim 1, wherein the compound is represented by Formula XXI: 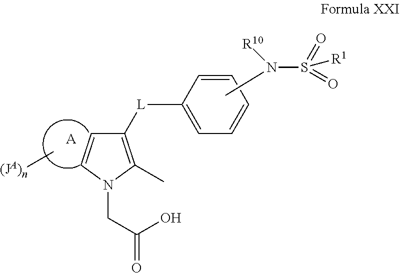
wherein:
Ring A is selected from a cyclopentane ring, a cycloheptane ring, a cyclohexane ring, a tetrahydropyran ring or a piperidine ring, each of them fused to the pyrrole ring;
each J^{A} is selected from ═O, ═NO—(C₁₋₄ alkyl), ═NO-(benzyl) or methyl;
n is 0, 2 or 3;
L is —CH₂— or —S—;
R¹⁰ is selected from hydrogen, methyl, ethyl or benzyl and
R¹ is selected from
(a) phenyl, optionally substituted by 0 to 2 instances of R⁸;
(b) cyclopropyl ring; or
(c) N-pyrrolidinyl, N-piperidinyl, N-morpholinyl, N-piperazinyl;

40. The compound of claim 39, represented by Formula XXII or Formula XXIII:

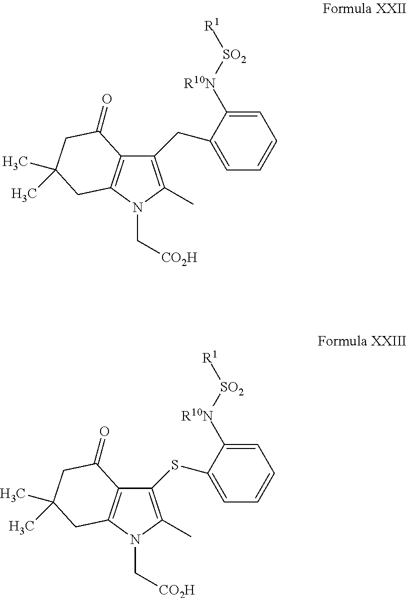

41. A compound selected from the compounds depicted in Table 1.

42. A composition comprising a pharmaceutically acceptable carrier and a compound according to claim 1.

43. A composition comprising a compound according to claim 42, further comprising one or more therapeutic agents selected from: inactivating antibodies to interleukins; soluble chemokine receptors; a chemokine receptor modulators; histamine HI receptor antagonists or antihistamines; leukotriene D4 receptor antagonists or leukotriene antagonists or a LTD4 antagonists; PGD2 receptor antagonists; VLA-4 antagonists; corticosteroids; immunosuppressants; non-steroidal anti-asthmatics, non-steroidal antiinflammatory agents (NSAIDs); cyclooxygenase-2 (COX-2) inhibitors; inhibitors of phosphodiesterase type IV (PDE-IV); opioid analgesics; antithrombotic agents; warfarin derivatives, β-blockers; β-adrenergic agonists; ACE inhibitors; vasodilators; anti-diabetic agents; preparations of interferon beta; gold compounds such as auranofin and aurothioglucose; TNF inhibitors; multiple sclerosis therapeutic agents; 5-aminosalicylic acid and prodrugs thereof; DNA-alkylating agents; antimetabolites; microtubule disruptors; DNA intercalators; DNA synthesis inhibitors; DNA cross-linking agents; hormone therapy; or cytostatic agents; and a pharmaceutically acceptable carrier.

44. A method for treating a patient suffering from a disease or disorder involving the CRTH2 receptor comprising administering to said patient a therapeutically effective amount of a compound according to claim 1.

45. The method according to claim 44, wherein said disease or disorder is asthma, atopic dermatitis, allergic rhinitis, allergy, Grave's Disease, acute rhinitis, hatrophic rhinitis or chronic rhinitis, rhinitis caseosa, hypertrophic rhinitis, rhinitis purulenta, rhinitis sicca, rhinitis medicamentosa, membranous rhinitis, croupous rhinitis, fibrinous rhinitis, pseudomembranous rhinitis, scrofulous rhinitis, perennial allergic rhinitis, seasonal rhinitis, rhinitis nervosa, vasomotor rhinitis, antitussive activity, bronchial asthma, allergic asthma, intrinsic asthma, extrinsic asthma, dust asthma, chronic asthma, inveterate asthma, late asthma, airway hyper-responsiveness, bronchitis, chronic bronchitis, eosinophilic bronchitis, chronic inflammatory diseases of the lung which result in interstitial fibrosis, interstitial lung diseases (ILD), idiopathic pulmonary fibrosis, ILD associated with rheumatoid arthritis, scleroderma lung disease, chronic obstructive pulmonary disease (COPD), chronic sinusitis, conjunctivitis, allergic conjunctivitis, cystic fibrosis, fanner's lung, fibroid lung, hypersensitivity lung disease, hypersensitivity pneumonitis, idiopathic interstitial pneumonia, nasal congestion, nasal polyposis, otitis media, chronic cough associated with inflammation, systemic anaphylaxis, hypersensitivity responses, drug allergies, insect sting allergies, food related allergies, food-related allergies with symptoms of migraine, rhinitis or eczema, arthritis, rheumatic arthritis, infectious arthritis, autoimmune arthritis, seronegative arthritis, spondyloarthropathy, ankylosing spondylitis, psoriatic arthritis, Reiter's disease, osteoarthritis, systemic sclerosis, psoriasis, atopical dermatitis, contact dermatitis, seborrheic dermatitis, cutaneous eosinophilias, chronic skin ulcers, cutaneous lupus erythematosus, contact hypersensitivity, allergic contact dermatitis, eosinophilic folliculitis, Coeliac disease, cholecystitis, Crohn's disease, enteritis, eosinophilic gastroenteritis, eosinophilic esophagitis, enteropathy associated with seronegative arthropathies, gastritis, inflammatory bowel disease, irritable bowel disease, acute and chronic allograft rejection following solid organ transplant, chronic graft versus host disease, skin graft rejection, bone marrow transplant rejection, inflammation, hyperalgesia, allodynia, neuropathic pain, lupus erythematosus; systemic lupus, erythematosus; Hashimoto's thyroiditis, Grave's disease, type I diabetes, eosinophilia fasciitis, hyper IgE syndrome, idiopathic thrombocytopenia pupura; post-operative adhesions, ischemic/reperfusion injury in the heart, brain, peripheral limb hepatitis, mastocytosis, mastitis, vaginitis, vasculitis, myositis, basophilic leukemia, basophilic leukocytosis, or Churg-Strauss syndrome.

46. The method according to claim 45 wherein the disease or disorder is asthma or an asthma attack.

47. The method according to claim 45 wherein the disease or disorder is allergic rhinitis.

48. The method according to claim 45 wherein the disease or disorder is Chronic Obstructive Pulmonary Disease.

49. The method according to claim 45 wherein the disease or disorder is neuropathic pain.

50. The method according to claim 45 wherein the disease or disorder is atopic dermatitis.

51. The method according to claim 45 wherein the disease or disorder is allergic conjunctivitis.

52. The method according to claim 45 wherein the disease or disorder is a gastrointestinal tract related disease or disorder selected from Crohn's disease, eosinophilic gastroenteritis, eosinophilic esophagitis, inflammatory bowel disease or irritable bowel disease.

53. The method according to claim 45, further comprising administering to said patient one or more therapeutic agents selected from: inactivating antibodies to interleukins; soluble chemokine receptors; a chemokine receptor modulators; histamine HI receptor antagonists or antihistamines; leukotriene D4 receptor antagonists or leukotriene antagonists or a LTD4 antagonists; PGD2 receptor antagonists; VLA-4 antagonists; corticosteroids; immunosuppressants; non-steroidal anti-asthmatics, non-steroidal antiinflammatory agents (NSAIDs); cyclooxygenase-2 (COX-2) inhibitors; inhibitors of phosphodiesterase type IV (PDE-IV); opioid analgesics; antithrombotic agents; warfarin derivatives, β-blockers; β-adrenergic agonists; ACE inhibitors; vasodilators; anti-diabetic agents; preparations of interferon beta; gold compounds such as auranofin and aurothioglucose; TNF inhibitors; multiple sclerosis therapeutic agents; 5-aminosalicylic acid and prodrugs thereof; DNA-alkylating agents; antimetabolites; microtubule disruptors; DNA intercalators; DNA synthesis inhibitors; DNA cross-linking agents; hormone therapy; or cytostatic agents.

54. The compound of claim 16, wherein L′ is attached ortho or para to L on ring B.

55. The compound of claim 35, wherein L′ is —SO₂— and ring B is a thiophenyl ring, a pyridyl ring or an indolyl ring.
